(19) United States
(12) Patent Application Publication
(10) Pub. No.: US 2013/0202646 A1
(43) Pub. Date: Aug. 8, 2013

(54) COMPOSITIONS AND METHODS FOR MODULATING AUTOPHAGY

(71) Applicant: Roberta A Gottlieb, Solana Beach, CA (US)

(72) Inventor: Roberta A Gottlieb, Solana Beach, CA (US)

(73) Assignee: SAN DIEGO STATE UNIVERSITY (SDSU) FOUNDATION, San Diego, CA (US)

(21) Appl. No.: 13/580,867

(22) PCT Filed: Feb. 25, 2011
(86) PCT No.: PCT/US2011/026299

§ 371 Date: Oct. 10, 2012

Related U.S. Application Data
(60) Provisional application No. 61/308,257, filed on Feb. 25, 2010.

(51) Int. Cl. C07K 14/47

(52) U.S. Cl. CPC C07K 14/4702
USPC 424/278.1; 536/23.4; 435/320.1; 530/350; 435/354; 514/21.2; 435/455; 514/44 R; 514/19.3; 514/4.8; 514/17.7; 514/3.7; 514/2.4; 514/4.4

(57) ABSTRACT

In alternative embodiments, the invention provides cell-permeable recombinant or synthetic proteins to modulate autophagy, including a Tat-Atg5K130R (inhibitor of autophagy) and a Tat-Beclin 1 (stimulant or activator of autophagy), and nucleic acids expressing them and methods for making and using them, e.g., to treat conditions and disorders responsive to autophagy modulation (e.g., where autophagy is dysregulated), including neurodegeneration, cystic fibrosis, cancer, heart failure, diabetes, obesity, sarcopenia, aging, ischemia/reperfusion, inflammatory disorders including Crohns, ulcerative colitis, biliary cirrhosis, lysosomal storage diseases, infectious diseases associated with intracellular pathogens including viruses, bacteria, and parasites such as Trypanosomes and malaria.

### RELATED APPLICATIONS

This application incorporates by reference and claims the benefit of priority under 35 U.S.C. §119(e) of U.S. Provisional Application No. 61/308,257, filed Feb. 25, 2010. The aforementioned application is explicitly incorporated herein by reference in its entirety and for all purposes.

### TECHNICAL FIELD

This invention generally relates to medicine, molecular biology and biochemistry. In alternative embodiments, the invention provides recombinant or synthetic proteins that can be administered to cells or animals to either stimulate or inhibit the process of autophagy. In particular, in one embodiment, the invention provides cell-permeable recombinant or synthetic proteins to modulate autophagy, including Ta-Atg5K130R (Tat-Atg5^{130R}) (inhibitor of autophagy) and Tat-Beclin1 (stimulant or activator of autophagy), and nucleic acids expressing them and methods for making and using them, e.g., to treat conditions and disorders responsive to authophagy modulation (e.g., where autophagy is dysregulated), including neurodegeneration, cancer, heart failure, obesity, sarcopenia, aging, ischemia/reperfusion, inflammatory disorders, and lysosomal storage diseases

### BACKGROUND

Efforts to understand the role of autophagy have been hampered by the lack of suitable reagents to monitor and manipulate autophagy. Current inhibitors of autophagy are very nonspecific.

Autophagy is dependent upon a number of proteins. One essential protein is Atg5, which contains a lysine residue at position 130, to which Atg12 is conjugated by an E3 ubiquitin ligase-like enzyme. Mutation of Lysine 130 prevents the conjugation reaction and thereby blocks the formation of autophagosomes. This was previously demonstrated to be the case in cells transiently transfected with mutant Atg5 (Atg5K130R).

Macroautophagy (referred to hereafter as autophagy) is the only means to remove dysfunctional organelles such as mitochondria and insoluble protein aggregates. The process is initiated by a number of stressors including starvation, oxidative stress, lipopolysaccharide exposure, and SI/R injury. Many studies of autophagy now rely on scoring the number of autophagosomes, which can be detected in transfected cells or transgenic animals expressing GFP (or the red fluorescent protein mCherry) fused to the protein LC3, which is incorporated into nascent autophagosomes. In the setting of myocardial sI/R injury, an increased prevalence of autophagosomes has been documented. In an in vivo model of myocardial ischemia, a reduction in stunning correlated with increased expression of Beclin 1 (an autophagy gene). Moreover, this group observed that within the tissue, cells with numerous autophagosomes were not TUNEL positive.

Effective therapies to reduce or prevent I/R injury in humans remain elusive despite a better understanding of the triggers, signaling pathways, and effectors that may be involved in preconditioning and postconditioning. These phenomena and the many pharmacological interventions that have been shown to condition the heart and confer protection appear to involve survival kinases, redox-sensitive mechanisms, PKC and mitochondrial K_{ATP} activation, and inhibition of mitochondrial permeability transition pore opening.

### SUMMARY

In alternative embodiments, the invention provides recombinant or synthetic proteins that can be administered to cells or animals to either stimulate or inhibit the process of autophagy.

In alternative embodiments, the invention provides isolated, recombinant or synthetic nucleic acids encoding a chimeric (hybrid) protein, wherein the chimeric (hybrid) protein comprises (or consists of):

(a) (i) a first domain comprising or consisting or: a peptide and/or a small molecule that confers cell permeability, for example: the protein transduction domain of an HIV Tat protein, e.g., the 11 amino acid protein transduction domain or HIV Tat; the protein transduction domain of Antennapedia; the Drosophila homeoprotein antennapedia transcription protein (AntHD); a poly-arginine sequence; a cationic N-terminal domain of a prion protein; a herpes simplex virus structural protein VP22; peptidomimetics and synthetic forms thereof; and, all equivalents and variants thereof capable of acting as a protein transduction domain, and

(ii) a second domain comprising or consisting of: a sequence comprising all or a subsequence of a wild type (non-mutated or manipulated) Atg5, or SEQ ID NO: 7; a sequence comprising all or a subsequence of an Atg5 with it lysine 130 mutated to an arginine or another (non-lysine) amino acid; a sequence comprising all or a subsequence of Beclin 1, e.g., a Beclin 1 fragment lacking the Bcl-2 binding domain such that it inhibits autophagy, or a peptidomimetic or synthetic form thereof, or an equivalent thereof;

for example, in one embodiment, the protein comprises or consists of a Tat-Atg5K130R (Tat-Atg5K130R (Tat-Atg5^{K130R}) (inhibitor of autophagy), a Tat-Beclin 1 (stimulates or increases autophagy), or a peptidomimetic or synthetic form thereof, or an equivalent thereof;

(b) the nucleic acid of (a), wherein the encoded chimeric (hybrid) protein further comprises a tag or detection moiety; or

(c) the nucleic acid of (a), wherein the tag or detection moiety comprises a tag for an antibody or an antigen binding fragment thereof (the antibody binding specifically to the tag or detection moiety, or the tag or detection moiety comprises a ligand, or the tag or detection moiety comprises a FLAG molecule or equivalent thereof; or

(d) the isolated, recombinant or synthetic nucleic acid of any of (a) to (c), wherein the nucleic acid encoding the chimeric (hybrid) protein is operatively linked to a transcriptional regulatory unit, or a promoter such as an inducible or constitutive promoter.

In alternative embodiments, one or both domains of a chimeric protein of the invention is isolated and/or derived from a bacterial, a yeast, an insect, or a mammalian cell or mammalian expression system, or an ex vivo artificial expression system; and may be purified by any suitable method, such as e.g., immuno- or affinity chromatography.

In alternative embodiments, the invention provides vectors, recombinant viruses, cloning vehicles, expression cassettes, cosmids or plasmids comprising (or consisting of) or having contained therein the isolated, recombinant or synthetic nucleic acid of the invention.

In alternative embodiments, the invention provides chimeric or hybrid polypeptides comprising (or consisting of): (a) the polypeptide encoded by the nucleic acid of the invention; or (b) the chimeric (hybrid) protein of (a), wherein the protein comprises a synthetic protein or peptide, recombinant protein or peptide, a peptidomimetic or a combination thereof.

In alternative embodiments, the invention provides chimeric or hybrid protein comprising (or consisting of):

(a) (i) a first domain comprising or consisting of: a peptide and/or a small molecule that confers cell permeability, for example: the protein transduction domain of an HIV Tat protein, e.g., the 11 amino acid protein transduction domain of HIV Tat; the protein transduction domain of Antennapedia; the Drosophila homeoprotein antennapedia transcription protein (AntHD); a poly-arginine sequence; a cationic N-terminal domain of a prion protein; a herpes simplex virus structural protein VP22; peptidomimetics and synthetic forms thereof; and, all equivalents and variants thereof capable of acting as a protein transduction domain, and

(ii) a second domain comprising or consisting of: a sequence comprising all or a subsequence of a wild type (non-mutated or manipulated) Atg5, or SEQ ID NO: 7; a sequence comprising all or a subsequence of an ATg5 with its lysine 130 mutated to an arginine or another (non-lysine) amino acid; a sequence comprising all or a subsequence of Beclin 1, e.g., a Beclin 1 fragment lacking the Bcl-2 binding domain such that it inhibits autophagy, or a peptidomimetic or synthetic form thereof, or an equivalent thereof;

for example, in one embodiment, the protein comprises or consists of a Tat-Atg5K130R (Tat-Atg5^{K130R}) (inhibitor of autophagy), a Tat-Beclin 1 (stimulates or increases autophagy), or a peptidomimetic or synthetic form thereof, or an equivalent thereof;

(b) the chimeric (hybrid) protein of (a), further comprising a tag or detection moiety, or an antibody or an antigen binding fragment thereof;

(c) the chimeric (hybrid) protein of (a) of (b), wherein the protein comprises (or consists of) a synthetic protein or peptide, recombinant protein or peptide, a peptidomimetic or a combination thereof.

In alternative embodiments, the invention provides cells comprising (a) the isolated, recombinant or synthetic nucleic acid of the invention; (b) the vector, recombinant virus, cloning vehicle, expression cassette, cosmid or plasmid of the invention; (c) the chimeric or hybrid polypeptide of the invention; or, (d) the cell of (a), (b) or (c), wherein the cell is a mammalian or a human cell.

In alternative embodiments, the invention provides pharmaceutical compositions or a formulations comprising the chimeric or hybrid protein of the invention; or the isolated, recombinant or synthetic nucleic acid of the invention; or the vector, recombinant virus, cloning vehicle, expression cassette, cosmid or plasmid of the invention; or the cell of the invention.

In alternative embodiments, the invention provides methods for modulating autophagy in a cell, comprising:

(a) providing: (i) a nucleic acid encoding the chimeric (hybrid) protein of the invention, or the nucleic acid of the invention, operatively linked to a transcriptional regulatory unit (e.g., a promoter, such as an inducible or constitutive promoter), or (ii) the vector, recombinant virus, cloning vehicle, expression cassette, cosmid or plasmid of the invention; and, a cell comprising an environment capable of supporting the expression of the chimeric (hybrid) protein by the nucleic acid; and

(b) inserting (e.g., transfecting or infecting) the nucleic acid, vector, recombinant virus, cloning vehicle, expression cassette, cosmid or plasmid of (a) into the cell.

In one embodiment, the transcriptional regulatory unit comprises a promoter, an inducible promoter or a constitutive promoter. The cell can be a mammalian cell, a monkey cell or a human cell. The nucleic acid, vector, recombinant virus, cloning vehicle, expression cassette, cosmid or plasmid can be inserted into the cell in vivo or in vitro.

In alternative embodiments, the invention provides methods for modulating autophagy in a cell, comprising:

(a) providing a chimeric or hybrid polypeptide of the invention, and

(b) inserting (e.g., transfecting or infecting) chimeric or hybrid polypeptide of (a) into the cell.

In alternative embodiments, the cell is a mammalian cell, a monkey cell or a human cell. In alternative embodiments, the chimeric or hybrid polypeptide is inserted into the cell in vivo or in vitro.

In alternative embodiments, the invention provides methods for ameliorating, preventing or treating a disease, a condition or a disorder responsive to autophagy modulation (e.g., where autophagy is dysregulated), comprising

(a) practicing any method of the invention; or

(b) administering to an individual in need thereof a sufficient amount of: the pharmaceutical composition or formulation of the invention; the chimeric or hybrid polypeptide of the invention; a nucleic acid encoding the chimeric (hybrid) protein of the invention; or the nucleic acid of the invention, operatively linked to a transcriptional regulatory unit (e.g., a promoter, such as an inducible or constitutive promoter); or the vector, recombinant virus, cloning vehicle, expression cassette, cosmid or plasmid of the invention.

In alternative embodiments, the disease, condition or disorder treated, prevented or ameliorated comprises neurodegeneration, cystic fibrosis, cancer, heart failure, diabetes, obesity, sarcopenia, aging, ischemia/reperfusion, inflammatory disorders including Crohns, ulcerative colitis, biliary cirrhosis, lysosomal storage diseases, infectious diseases associated with intracellular pathogens including viruses, bacteria, and parasites such as Trypanosomes and malaria.

In alternative embodiments, the autophagy is modulated in order to increase the efficacy of a vaccine. In alternative embodiments, the invention provides methods for increasing the efficacy of a vaccine by practicing any method of the invention; or administering to an individual in need thereof a sufficient amount of: the pharmaceutical composition or formulation of the invention; the chimeric or hybrid polypeptide of the invention; a nucleic acid encoding the chimeric (hybrid) protein of the invention; or the nucleic acid of the invention, operatively linked to a transcriptional regulatory unit (e.g., a promoter, such as an inducible or constitutive promoter); or the vector, recombinant virus, cloning vehicle, expression cassette, cosmid or plasmid of the invention.

The details of one or more embodiments of the invention are set forth in the accompanying drawings and the description below. Other features, objects, and advantages of the invention will be apparent from the description and drawings, and from the claims.

All publications, patents, patent applications, GenBank sequences and ATCC deposits, cited herein are hereby expressly incorporated by reference for all purposes.

### DESCRIPTION OF DRAWINGS

FIG. 1 illustrates adenosine receptor-selective effects on autophagy; and FIG. 1(A) graphically illustrates date where GFP-LC3 transfected HL-1 cells were treated for 120 min in full medium (FM) with various concentrations (0.001-10 μM) of CCPA; FIG. 1(B) graphically illustrates data where GFP-LC3-transfected HL-1 cells were treated with 100 nM CCPA for the indicated time, then fixed with paraformaldehyde and scored by fluorescence microscopy; FIG. 1(C) illustrates representative images of HL-1 cells expressing GFP-LC3, which is diffuse in quiescent cells and punctate in CCPA-treated cells (PC); FIG. 1(D) illustrates representative images of neonatal cardiomyocytes under control conditions or 10 min after administration of 100 nM CCPA; FIG. 1(E) illustrates representative images of adult cardiomyocytes under control conditions or 10 min after administration of 100 nM CCPA; FIG. 1(F) illustrates representative fluorescence microscropy images where transgenic mice expressing mCherry-LC3 under the αMHC promoter received an i.p. injection of saline or 1 mg/kg CCPA, then were sacrificed 30 min later and heart tissue was processed for fluorescence microscopy; as described in detail in Example 1, below.

FIG. 2 graphically illustrates data showing the effect of CCPA on autophagic flux under conditions of starvation or sI/R; HL-1 cells were infected with adv-GFP-LC3, treated with or without 100 nM CCPA in full medium (FM) for 10 min, then subjected either to starvation (amino acid deprivation in MKH) (Stv) for 3 hr, or simulated I/R (2 hr sI, 3 hr R; as described in detail in Example 1, below.

FIG. 3 graphically illustrates data showing the receptor-selective effect of CCPA on autophagy and cytoprotection; Adv-GFP-LC3 infected HL-1 cells were treated in full medium with the selective A1 receptor antagonist DPCPX for 30 min, followed by 100 nM CCPA for 10 min, and then cells were subjected to sI/R (2 hr sI, 3 hr R); the extent of autophagy was assessed by the intracellular distribution of GFP-LC3 by fluorescence microscopy as illustrated in FIG. 3(A), and cell death was measured by LDH release at the end of simulated ischemia as illustrated in FIG. 3(B) or by propidium iodide uptake at the end of reperfusion as illustrated in FIG. 3(C); as described in detail in Example 1, below.

FIG. 4 graphically illustrates data showing that CCPA signals autophagy through PLC: HL-1 cells infected with Adv-GFP-LC3 were treated with the PLC inhibitor U73122 (2 μM) for 15 min followed by CCPA for 10 min, then incubated in normoxic conditions or subjected to sI/R (2 hr sI, 3 hr R); autophagy was scored by fluorescence microscopy as illustrated in FIG. 4(A); the amount of LDH released to the medium was determined immediately after ischermia and compared to the total activity of control homogenate (100%) as illustrated in FIG. 4(B); as described in detail in Example 1, below.

FIG. 5 graphically illustrates data showing that CCPA signals autophagy through a rise in intracellular calcium; HL-1 cells were treated with 1 μM thapsigargin (TG) or 25 μM BAPTA-AM for 15 min followed by CCPA for 10 min; cells were washed in PBS and fixed and the intracellular distribution of GFP-LC3 was assessed by fluorescence microscopy; as described in detail in Example 1, below.

FIG. 6 graphically illustrates data showing that cytoprotection by CCPA is dependent upon autophagy: HL-1 cells were co-transfected with GFP-LC3 and the dominant negative autophagy protein Atg5^{K130R}; after 24 hr cells were treated for 10 min with CCPA followed by sI/R (2 hr sI, 3 hr R); the extent of autophagy was assessed by the intracellular distribution of GFP-LC3 by fluorescence microscopy as illustrated in FIG. 6(A); cytoprotection was assessed by measuring LDH released into the media at the end of ischemia as illustrated in FIG. 6(B) or by propidium iodide uptake as illustrated in FIG. 6(C); as described in detail in Example 1, below.

FIG. 7 graphically illustrates data showing that cytoprotection by CCPA requires autophagy in adult cardiomyocytes: adult rat cardiomyocytes were infected with GFP-LC3 adenovirus for 2 hours and washed with the plating medium; after 20 hr, cells were incubated with or without Tat-Atg5^{K130R} for 30 min followed by CCPA or vehicle for 10 min; cells were subjected to normoxia or simulated ischemia followed by 2 hr reperfusion, and autophagy was scored as the percentage of cells with numerous puncta as illustrated in FIG. 7(A); for determination of cell death, LDH release into the culture supernatant was measured at the end of simulated ischemia as illustrated in FIG. 7(B); as described in detail in Example 1, below.

FIG. 8 graphically illustrates data showing that receptor-selective stimulation of autophagy in delayed preconditioning: GFP-LC3 infected HL-1 cells were treated with the selective A1 receptor antagonist DPCPX of 30 min prior to CCPA exposure for 10 min followed by washout; after 24 hr, the cells were exposed to sI/R (2 hr sI, 3 hr R); the cells were fixed, and the extent of autophagy was assessed by the intracellular distribution of GFP-LC3 by fluorescence microscopy in normoxia and after sI/R as illustrated in FIG. 8(A); cell death was measured by LDH release at the end of ischemia as illustrated in FIG. 8(B); as described in detail in Example 1, below.

FIG. 9 graphically illustrates data showing the role of autophagy in delayed preconditioning: HL-1 cells were co-transfected with GFP-LC3 and the exemplary dominant negative Atg5^{K130R}; cells were treated with CCPA for 10 min, followed by washout; 20 hr later, cells were subjected to sI/R (2 hr sI, 3 hr R); the extent of autophagy was assessed by the intracellular distribution of GFP-LC3 by fluorescence microscopy as illustrated in FIG. 9(A) and cell death was measured by LDH release into the medium at the end of ischemia as illustrated in FIG. 9(B); as described in detail in Example 1, below.

FIG. 10 illustrates the effects of SUL on I/R injury in isolated perfused rat hearts: FIG. 10A graphically illustrates data where sulfaphenazole or vehicle was infused before 30 min of global no-flow ischemia, and coronary effluent was collected for the first 15 min of reperfusion for determination of CK release; FIG. 10B graphically illustrates data where hearts treated as above were reperfused for 120 min and infarct size was measured by TTC staining; FIG. 10C illustrates representative slices of TTC-stained hearts; FIGS. 10D, 10E and 10F graphically illustrate data showing that pre-ischemic SUL administration enhances recovery of function, as measured by recovery of developed pressure, dp/dtₘₐₓ, and dp/dtₘᵢₙ); as described in detail in Example 2, below.

FIG. 11 illustrates that SUL induces autophagy in rat and mouse hearts: FIG. 11A illustrates where rat hearts were perfused with vehicle or SUL for 30 min, and then fixed and immunostained for LC3 antibody (insert (a) and (b)); vehicle or SUL was administered by i.p. injection to mCherry-LC3 transgenic mice and hearts were removed for tissue processing 60 min later (insert (c) and (d)), FIG. 11B illustrates a representative Western blot to detect LC3-1 and LC3-II in rat hearts perfused with vehicle or SUL; FIG. 11C graphically illustrates quantification of LC3-II/LC3-I; FIG. 11D graphically illustrates quantification of autophagosomes (mCherry-LC3 puncta) in hearts of mice that received vehicle or SUL (*p<0.01, n=6); as described in detail in Example 2, below.

FIG. 12 illustrates the effect of SUL on PKC δtranslocation: FIG. 12A illustrates immunoblots of cytosol and particulate fractions of rat hearts 30 min after SUL infusion (Langendorff); FIG. 12B illustrates fluorescence micrograph of adult rat cardiomyocytes treated with SUL or vehicle (CON) for 15 min, then fixed and immunostained with antibody to PKC δ and α-actinin (inset shows a higher resolution field, N=nuclei; FIG. 12C graphically illustrates a pseudo-line scan derived from the myocytes shown in FIG. 12B, in which the fluorescence intensity (y axis; a.u., arbitrary units) is measured along a defined segment of the myocyte on the longitudinal axis (x axis); as described in detail in Example 2, below.

FIG. 13 illustrates the role of PKC in autophagy induction by SUL in rat cardiomyocytes: FIG. 13A illustrates isolated adult cardiocyocytes were infected with GFP-LC3 adenovirus; FIG. 13B graphically illustrates quantification of autophagy by percentage of cells displaying numerous puncta; as described in detail in Example 2, below.

FIG. 14 illustrates the role of PKC in autophagy and cardioprotection in isolated perfused rat hearts: FIG. 14A illustrates hearts sections, were hearts were treated with chelerythrine with or without SUL, then subjected to I/R and stained with TTC for infarct size determination; FIG. 14B graphically illustrates quantification of infarct size after administration of chelerythrine is shown; FIG. 14C graphically illustrates quantification of autophagy in perfused hearts treated as indicated and measured by cadaverine dye binding assay; as described in detail in Example 2, below

FIG. 15 illustrates the effects of Tat-Atg5^{K130R} and SUL on autophagy in isolated perfused rat hearts FIG. 15A graphically illustrates a protocol for Langendorff perfusion; FIG. 15B illustrates immunofluorescence of Tat-Atg5^{K130R} in cardiomyocytes as detected by anti-HA antibody (green immunofluroescence), BODIPY-TR™-cadaverine incorporation into autophagosomes (red fluorescence) was increased by SUL administration (reflecting increased autophagy) and diminished by pre-treatment with Tat-Atg5^{K130R}; FIG. 15C illustrates quantification of autophagy by cadaverine dye binding in heart tissue; as described in detail in Example 2, below.

FIG. 16 illustrates induction of autophagy by SUL is abolished by administration of Tat-Atg5^{K130R}; rat hearts were perfused with Tat-Atg5^{K130R} as indicated in FIG. 15A, followed by addition of SUL or vehicle to perfusion buffer and treatment as indicated; FIG. 16A graphically illustrates quantification of the LC3-II/LC3-I ratio from Western blots; FIG. 16B graphically illustrates quantification of autophagy by cadaverine binding assay; FIG. 16C graphically illustrates hearts treated as above were reperfused for 120 min and infarct size was determined by TTC staining; as described in detail in Example 2, below.

FIG. 17 illustrates that sulfaphenazole (Sul) reduces infarct size when given at reperfusion, but the protection is lost if autophagy is blocked with Tat-Atg5^{K130R}; FIG. 17A graphically illustrates the protocol; FIG. 17B illustrates representative TTC-stained sections are shown; FIG. 17C graphically illustrates the quantitation, as based on 3 hearts per condition; as described in detail in Example 2, below.

FIG. 18 graphically illustrates data showing that Tat proteins can modulate autophagy: HL-b 1cells were transfected with LC3GFP and then treated with Tat-Atg5^{K130R} (which inhibits autophagy) or Tat-Beclin 1 (which stimulates autophagy); as described in detail in Example 2, below.

Like reference symbols in the various drawings indicate like elements.

### DETAILED DESCRIPTION

In alternative embodiments, the invention provides cell-permeable recombinant or synthetic proteins to modulate autophagy, including Tat-Atg5K130R (inhibitor of autophagy) and Tat-Beclin 1 (stimulant or activator of autophagy), and nucleic acids expressing them and methods for making and using them, e.g., to treat conditions and disorders responsive to autophagy modulation (e.g., where autophagy is dysregulated), including neurodegeneration, cancer, heart failure, obesity, sarcopenia, aging, ischemia/reperfusion, inflammatory disorders, and lysosomal storage diseases.

In alternative embodiments, the cell-permeable recombinant or synthetic proteins of the invention are administered to cells, tissues, organs, or whole animals, to specifically interfere with autophagy.

Beclin 1 is important for initiating autophagy, and we have shown that overexpression can stimulate autophagy. We generated a cell-permeable recombinant protein that can be administered to cells, tissues, organs, or whole animals to stimulate autophagy. In alternative embodiments, this can offers advantages over small molecule agents to stimulate autophagy, because these drugs often have multiple effects that may be unrelated to autophagy.

### In Vivo or In Situ Delivery

In addition to cellular and nucleic acid approaches, chimeric molecules (e.g., proteins) used to practice this invention can be delivered directly to an affected tissue or organ, e.g., to the brain, or to cardiac or other circulatory tissues. Because Atg5K130R (Atg5^{K130R}) and Beclin 1 act intracellulary, in alternative embodiment the invention utilizes a delivery strategy to facilitate intracellular delivery. In alternative embodiments, chimeric molecules (e.g., proteins) used to practice this invention are delivered to a variety of cells, tissues, organs to either stimulate or inhibit the process of autophagy: e.g., in one embodiment, to inhibit autophagy, such as Atg5K130R (Tat-Atg5^{K130R}), or a Beclin 1 to stimulate or activate autophagy.

One technique that can be used is to provide the Atg5K130R (Atg5^{K130R}) and/or Beclin 1 (or equivalents thereof) in a vehicle that in taken up by or that fuses with a target cell. Thus, for example, molecules of the invention can be encapsulated within a liposome or other vehicle, as described in more detail above in connection with polynucleotide delivery to cells.

Alternatively, the Atg5K130R (Tat-Atg5^{K130R}) and/or Beclin 1 (or equivalents thereof) may be linked to a transduction domain, such as TAT protein. In some embodiments, the Atg5K130R (Tat-Atg5^{K130R}) and/or Beclin 1 (or equivalents thereof) can be operably linked to a transduction moiety, such as a synthetic or non-synthetic peptide transduction domain (PTD), Cell penetrating peptide (CPP), a cationic polymer, an antibody, a cholesterol or cholesterol derivative, a Vitamin E compound, a tocol, a tocotrienol, a tocopherol, glucose, receptor ligand or the like, to further facilitate the uptake of the Atg5K130R (Atg5^{K130R}) and/or Beclin 1 (or equivalents thereof) by cells.

A number of protein transduction domains/peptides are known in the art and facilitate uptake of heterologous molecules linked to the transduction domains (e.g., cargo molecules). Such peptide transduction domains (PTD's) facilitate uptake through a process referred to as macropinocytosis. Macropinocytosis is a nonselective form of endocytosis that all cells perform.

In alternative embodiments, exemplary peptide transduction domains (PTD's) are derived from the Drosophila homeoprotein antennapedia transcription protein (AntHD) (Joliot et al., New Biol. 3:1121-23, 1991; Joliot et al., Proc. Natl. Acad. Sci. USA, 88:1864-8, 1991; Le Roux et al., Proc. Natl. Acad. Sci. USA, 90:9120-4, 1993), the herpes simplex virus structural protein VP212 (Elliott and O'Hare, Cell 88:223-33, 1997), the HIV-1 transcriptional activator TAT protein (Green and Loewenstein, Cell 55:1179-1188, 1988; Frankel and Pabo, Cell 55:1189-1193, 1988), the cationic N-terminal domain of prion proteins; a herpes simplex virus structural protein VP22; and equivalents thereof.

In alternative embodiments, the peptide transduction domain increases uptake of the Atg5K130R (Tat-Atg5^{K130R}) and/or Beclin 1 (or equivalents thereof); which in some embodiment is fused in a receptor independent fashion, and can be capable of transducing a wide range of cell types, and can exhibit minimal or no toxicity (see e.g., Nagahara et al., Nat. Med. 4:1449-52, 1998). In alternative embodiments, the peptide transduction domain used to practice the invention include peptide transduction domains that have been shown to facilitate uptake of DNA (see e.g., Abu-Amer, supra), antisense oligonucleotides (see e.g., Astriab-Fisher et al., Pharm. Res, 19:744-54, 2002), small molecules (see e.g., Polyakov et al., Bioconjug. Chem. 11:762-71, 2000) and even inorganic 40 nanometer iron particles (see e.g., Dodd et al., J. Immunol. Methods 256:89-105, 2001; Wunderbaldinger et al., Bioconjug, Chem. 13:264-8, 2002; Lewin et al., Nat. Biotechnol, 18:410-4, 2000; Josephson et al., Bioconjug., Chem. 10:186-91, 1999).

Fusion proteins of the invention with such trans-cellular delivery proteins can be readily constructed using known molecular biology techniques.

In alternative embodiments, any of the polynucleotides encoding the Atg5K130R (Atg5^{K130R}) and/or Beclin 1 (or equivalents thereof) can be linked to any of these transduction domains to facilitate transduction of those polynucleotides into a target cell or organ or tissue in vivo or in vitro.

### Chimeric/Hybrid Polypeptides

In alternative embodiments the invention provides chimeric or hybrid protein comprising (or consisting of) a first domain comprising or consisting of: a peptide and/or a small molecule that confers cell permeability, and a second domain comprising or consisting of: an autophagy-modulating sequence.

For example, in one embodiment, an exemplary chimeric or hybrid protein-encoding nucleic acid of the invention consists of or comprises a DNA sequence comprising TAT-HA ATG5(K130R, a mouse ATG5 with the K130R mutation:

SEQ ID NO: 2
ATGCGGGGTTCTCATCATCATCATCATCATGGTATGGCTAGCATGACTGGTGGACAGCAAATGGGTCGGG
ATCTGTACGACGATGACGATAAGGATCGATGGGGATCCAAGCTTGGCTACGGCCGCAAGAAACGCCGCCA
GCGCCGCCGCGGTGGATCCACCATGTCCGGCTATCCATATGACGTCCCAGACTATGCTGGCTCCATGGCC
GGTACCATGACAGATGACAAAGATGTGCTTCGAGATGTGTGGTTTGGACGAATTCCAACTTGCTTTACTC
TCTATCAGGATGAGATAACTGAAAGAGAAGCAGAACCATACTATTTGCTTTTGCCAAGAGTCAGCTATTT
GACGTTGGTAACTGACAAAGTGAAAAAGCACTTTCAGAAGGTTATGAGACAAGAAGATGTTAGTGAGATA
TGGTTTGAATATGAAGGCACACCCCTGAAATGGCATTATCCAATTGGTTTACTATTTGATCTTCTTGCAT
CAAGTTCAGCTCTTCCTTGGAACATCACAGTACATTTCAAGAGTTTTCCAGAAAAGGACCTTCTACACTG
TCCATCCAAGGATGCGGTTGAGGCTCACTTTATGTCGTGTATGAGAGAAGCTGATGCTTTAAAGCATAAA
AGTCAAGTGATCAACGAAATGCAGAAAAAAGACCACAAGCAGCTCTGGATGGGACTGCAGAATGACAGAT
TTGACCAGTTTTGGGCCATCAACCGGAAACTCATGGAATATCCTCCAGAAGAAAATGGATTTCGTTATAT
CCCCTTTAGAATATATCAGACCACGACGGAGCGGCCTTTCATCCAGAAGCTGTTCCGGCCTGTGGCCGCA
GATGGACAGCTGCACACACTTGGAGATCTCCTCAGAGAAGTCTGTCCTTCCGCAGTCGCCCCTGAAGATG
GAGAGAAGAGGAGCCAGGTGATGATTCACGGGATAGAGCCAATGCTGGAAACCCCTCTGCAGTGGCTGAG
CGAGCATCTGAGCTACCCAGATAACTTTCTTCATATTAGCATTGTCCCCCAGCCAACAGATTGA

First ATG of TAT and ATG5 are underlined in blue

6 his underlined in red

11 AA TAT underlined in green

HA tag underlined in brown

Mutation at Amino Acid 130 K to R in Brown

Stop codon in blue

In one embodiment, an exemplary chimeric or hybrid protein-encoding nucleic acid of the invention consists of or comprises the Amino Acid Translation of the mouse TAT ATG5K130R):

SEQ ID NO: 1
MRGSHHHHHHGMASMTGGQQMGRDLYDDDDKDRWGSKLGYGRKKRRQRRRGGSTMSGYPYDVPDYAGSMA
GTMTDDKDVLRDVWFGRIPTCFTLYQDEITEREAEPYYLLLPRVSYLTLVTDKVKKHFQKWMRQEDVSEI
WFEYEGTPLKWHYPIGLLFDLLASSSALPWNITVHFKSFPEKDLLHCPSKDAVEAHFMSCMREADALKHK
SQVINEMQKKDHKQLWMGLQNDRFDQFWAINRKLMEYPPEENGFRYIPFRIYQTTTERPFIQKLFRPVAA
DGQLHTLGDLLREVCPSAVAPEDGEKRSQVMIHGIEPMLETPLQWLSEHLSYPDNFLHISIVPQPTD*

6 his underlined in red

11 AA TAT underlined in green

HA tag underlined in brown

AA 130 mutation to Arginine, R, in Blue

In one embodiment, a wild type ATG5 is used, e.g., for the mouse WT, the brown AGA would be AAA and in the amino acid sequence the blue R (arginine, art) would be K (lysine, lys).

In one embodiment, an exemplary chimeric or hybrid protein-encoding nucleic acid of the invention consists of or comprises a DNA sequence comprising TAT-HA Beclin 1, a Rat Beclin 1 sequence:

SEQ ID NO: 4
ATGCGGGGTTCTCATCATCATCATCATCATGGTATGGCTAGCATGACTGGTGGACAGCAAATGGGTCGGG
ATCTGTACGACGATGACGATAAGGATCGATGGGGATCCAAGCTTGGCTACGGCCGCAAGAAACGCCGCCA
GCGCCGCCGCGGTGGATCCACCATGTCCGGCTATCCATATGACGTCCCAGACTATGCTGGCTCCATGGCC
GGTACCGGTCTCGAGATGGAGGGGTCTAAGGCGTCCAGCAGCACCATGCAGGTGAGCTTCGTGTGCCAGC
GCTGTAGCCAGCCTCTGAAACTGGACACGAGCTTCAAGATCCTGGACCGAGTGACCATTCAGGAACTCAC
AGCTCCATTACTTACCACAGCCCAGGCGAAACCAGGAGAGAGCCAGGAGGAAGAGGCTAACTCAGGAGAG
GAGCCATTTATTGAAACTCGCCAGGATGGTGTCTCTCGAAGATTCATCCCCCCAGCCAGGATGATGTCTA
CAGAAAGTGCTAATAGCTTCACTCTGATCGGGGAGGCATCTGATGGTGGCACCATGGAGAACCTCAGCCG
GAGACTCAAGGTCACTGGAGACCTTTTTGACATCATGTCTGGCCAGACAGATGTGGATCACCCACTGTGT
GAGAAATGCACAGATACTCTTTTAGACCAGCTGGACACTCAGCTCAATGTTACTGAAAACGAGTGTCAGA
ACTACAAACGCTGTTTGGAGATGTTGGAGCAAATGAATGAGGGCGACAGTGAACAGCTACAGAGGGAGCT
GAAGGAGTTGGCCTTGGAGGAGGAGAGGCTGATCCAGGAGCTGGAAGATGTGGAAAAAAACCGAAAGGTG
GTGGCAGAAAACCTGGAGAAGGTCCAGGCTGAGGCGGAGAGACTGGACCAGGAGGAAGCTCAGTACCAGC
GAGAATATAGTGAATTTAAAAGGCAGCAGCTGGAGCTGGATGATGAGCTCAAGAGTGTAGAGAACCAGAT
GCGCTATGCCCAGATGCAGCTGGACAAGCTCAAGAAAACCAATGTCTTCAATGCGACCTTCCATATCTGG
CACAGCGGACAATTTGGCACGATCAATAATTTCAGACTGGGTCGCTTGCCCAGTGCTCCTGTGGAATGGA
ATGAAATCAATGCTGCCTGGGGCCAGACAGTGTTGTTGCTCCATGCTTTGGCCAATAAGATGGGTCTGAA
GAGTTGCCGTTGTACTGTTCTGGGGGTTTGCGGTTTTTCTGGGACAACAAGTTTGACCATGCAATGGTAG
CTTTTCTGGACTGTGTGCAGCAGTTCAAAGAAGAGGTGGAAAAAGGAGAGACTCGATTTTGTCTTCCGTA
CAGGATGGACGTGGAGAAAGGCAAGATTGAAGACACTGGAGGCAGTGGCGGCTCCTATTCCATCAAAACC
CAGTTTAACTCTGAGGAGCAGTGGACAAAGGCGCTCAAGTTCATGCTGACGAATCTCAAGTGGGGTCTTG
CTTGGGTGTCCTCACAGTTCTATAACAAGTGA

First ATG of TAT and Beclin underlined in blue

6 his underlined in red

11 AA TAT underlined in green

HA tag underlined in brown

Stop codon in blue

In once embodiment, an exemplary chimeric or hybrid protein-encoding nucleic acid of the invention consists of or comprises the Amino Acid Translation of the TAT Beclin 1 from first ATG of TAT domain:

SEQ ID NO: 3
MRGSHHHHHHGMASMTGGQQMGRDLYDDDDKDRWGSKLGYGRKKRRQRRRGGSTMSGYPYDVPDYAGSMA
GTGLEMEGSKASSSTMQVSFVCQRCSQPLKLDTSFKILDRVTIQELTAPLLTTAQAKPGESQEEEANSGE
EPFIETRQDGVSRRFIPPARMMSTESANSFTLIGEASDGGTMENLSRRLKVTGDLFDIMSGQTDVDHPLC
EECTDTLLDQLDTQLNVTENECQNYKRCLEMLEQMNEGDSEQLQRELKELALEEERLIQELEDVEKNRKV
VAENLEKVQAEAERLDQEEAQYQREYSEFKRQQLELDDELKSVENQMRYAQMQLDKLKKTNVFNATFHIW
HSGQFGTINNFRLGRLPSAPVEWNEINAAWGQTVLLLHALANKMGLKFQRYRLVPYGNHSYLESLTDKSK
ELPLYCSGGLRFFWDNKFDHAMVAFLDCVQQFKEEVEKGETRFCLPYRMDVEKGKIEDTGGSGGSYSIKT
QFNSEEQWTKALKFMLTNLKWGLAWVSSQFYNK*

6 his underlined in red

11 AA TAT underlined in green

HA tag underlined in brown

In alternative embodiments, human equivalents of wild type ATG5 and Beclin 1, and modified ATG5, are used to practice this invention. For example, in one embodiment, a sequence used for human therapy would not include an HA tag or a 6-His tag but would include a Tat transduction domain (green), as noted below, and a Lys→Arg mutation highlighted:

SEQ ID NO: 5
MRGSYGRKKRRQRRRGGSMTDDKDVLRD VWFGRIPTCF TLYQDEITER EAEPYYLLLP
RVSYLTLVTD KVKKHFQKVM RQEDISEIWF EYEGTPLKWH YPIGLLFDLL ASSSALPWNI
TVHFKSFPEK DLLHCPSKDA IEAHFMSCMR EADALKHKSQ VINEMQKKDH KQLWMGLQND
RFDQFWAINR KLMEYPAEEN GFRYIPFRIY QTTTERPFIQ KLFRPVAADG QLHTLGDLLK
EVCPSAIDPE DGEKKNQVMI HGIEPMLETP LQWLSEHLSY PDNLLHISII PQPTD*

In alternative embodiments, a wild type human Atg5 nucleic acid sequence used to practice the invention is: (in one embodiment, not including the added components of Tat protein transduction domain or spacers):

```
SEQ ID NO: 6
      1 gtgacgtcat ctccgggcgc cgagggtgac tggacttgtg gtgcgctgcc agggctccgc
    61 agcgttgccg gttgtattcg ctggatacca gagggcggaa gtgcagcagg gttcagctcc
  121 gacctccgcg ccggtgcttt ttgcggctgc gcgggcttcc tggagtcctg ctaccgcgtc
  181 cccgcaggac agtgtgtcag gcgggcagct tgccccgccg ccccaccgga gcgcggaatc
  241 tgggcgtccc caccagtgcg gggagccgga aggaggagcc atagcttgga gtaggtttgg
  301 ctttggttga aataagaatt tagcctgtat gtactgcttt aactcctgga agaatgacag
  361 atgacaaaga tgtgcttcga gatgtgtggt ttggacgaat tccaacttgt ttcacgctat
  421 atcaggatga gataactgaa agggaagcag aaccatacta tttgcttttg ccaagagtaa
  481 gttatttgac gttggtaact gacaaagtga aaaagcactt tcagaaggtt atgagacaag
  541 aagacattag tgagatatgg tttgaatatg aaggcacacc actgaaatgg cattatccaa
  601 ttggtttgct atttgatctt cttgcatcaa gttcagctct tccttggaac atcacagtac
  661 ttggtttgct atttgatctt cttgcatcaa gttcagctct tccttggaac atcacagtac
  721 ctcattttat gtcatgtatg aaagaagctg atgctttaaa acataaaagt caagtaatca
  781 atgaaatgca gaaaaaagat cacaagcaac tctggatggg attgcaaaat gacagatttg
  841 accagttttg ggccatcaat cggaaactca tggaatatcc tgcagaagaa aatggatttc
  901 gttatatccc ctttagaata tatcagacaa cgactgaaag acctttcatt cagaagctgt
  961 ttcgtcctgt ggctgcagat ggacagttgc acacactagg agatctcctc aaagaagttt
1021 gtccttctgc tattgatcct gaagatgggg aaaaaaagaa tcaagtgatg attcatggaa
1081 ttgagccaat gttggaaaca cctctgcagt ggctgagtga acatctgagc tacccggata
1141 attttcttca tattagtatc atcccacagc caacagattg aaggatcaac tatttgcctg
1201 aacagaatca tccttaaatg ggatttatca gagcatgtca cccttttgct tcaatcaggt
1261 ttggtggagg caacctgacc agaaacactt cgctgctgca agccagacag gaaaaagatt
1321 ccatgtcaga taaggcaact gggctggtct tactttgcat cacctctgct ttcctccact
1381 gccatcatta aacctcagct gtgacatgaa agacttaccg gaccactgaa ggtcttctgt
1441 aaaatataat gaagctgaaa cctttggcct aagaagaaaa tggaagtatg tgccactcga
1501 tttgtatttc tgattaacaa ataaacaggg gtatttccta aggtgaccat ggttgaactt
1561 tagctcatga aagtggaaac attggtttaa ttttcaagag aattaagaaa gtaaaagaga
1621 aattctgtta tcaataactt gcaagtaatt ttttgtaaaa gattgaatta cagtaaaccc
1681 atctttcctt aacgaaaatt tcctatgttt acagtctgtc tattggtatg caatcttgta
1741 actttgataa tgaacagtga gagattttta aataaagcct ctaaatatgt tttgtcattt
1801 aataacatac agttttgtca cttttcaagt actttctgac tcacatacag tagatcactt
1861 tttactctgt gttaccattt tgactggtcg tcattggcat ggggtggata tagggcatag
1921 gattacttgt ctcagaagct gtcatagaat ttcttgctgc caattaaaaa acctgtgttc
1981 tttacacact acacgtataa atattgtaac tgttcatctt tgttgtttta tcactgtaag
2041 cctgtcaaat catagtatcc taagcatctg taaatgctaa ttttgcattt ttggaaaaac
2101 ccattccttc caagctagtg tttttcattg gctccaggtc taatttttca ctgtggtccc
2161 tggcagccag tcttttgaag tttaaagatt acctgtctct tgactgcagt accttttctt
2221 taatttttac caaaaatatc cagaggttac tggagttctt attcaatata aggaaagttt
2281 gtcgcacttt attaccaagc ctctgggatt ttaccagtca aacatatttg tgcattacat
2341 ttcatttctt gtgagctagc tggctgtcca tattgaatgt tgacccattt gagtacgcta
2401 aaaggcttac agtatcagac acgatcatgg ttttagatcc cataataaaa atgaatgttt
2461 ttcttataaa aaattataca aatgctgaag tgagattcta ctattgttca ttgcttcctt
2521 ttctttttcc ttttgcgatt ttcactgatt aatagcacat ttcttcacaa aattagataa
2581 agttggtcaa agaccagata ttctggaatg gaaattgtaa agcttaatca aaaagaatag
2641 ccagtacagc atacaatctc agaaacttag aagcaagtag aaaataattg gttgatgtaa
2701 acgaaagtgc cattttagta aaggcaggaa aaaaatagca atatttgagt tatgtaagga
2761 taaaaaatcc actgacttgt atttttgcac aagaggctgg tctgaatatg attgttcaca
2821 ttaagagtgt ttattcgtcg gttcattttg gggattttcc cccttgatgt tttgacagat
2881 tgaagtgagc tttagtgagc aaaaggatca gaatgcaggg aacactaagc tgtgatgaag
2941 aaagtgtggt aaaaagccag agtagtttta tacagacaaa accagtgtca ggcctttgca
3001 gtaggcttga gtgaacttct gatctagatt tgaaagtaaa ttttatgaag acattgccca
3061 tttttacttc ctcattcatt attgtaccag catcatagct ttattactct aatcccaggt
3121 aagtcaagcc tacaatgccc tagaggaaga gtaaaaccag aaattcatgc tggcttaaat
3181 aatctatttt tgtttctttt catttgaata tttaaatttt atggtttatt aaaaaattaa
3241 ataa
```

In alternative embodiments, a wild type human Atg5 protein used to practice the invention is: (in one embodiment, not including the added components of Tat protein transduction domain or spacers):

SEQ ID NO: 7
mhypigllfd llasssalpw nitvhfksfp ekdllhcpsk daieahfmsc mkeadalkhk
sqvinemqkk dhkqlwmglg ndrfdqfwai nrklmeypae engfryipfr iyqttterpf
iqklfrpvaa dgqlhtlgdl lkevcpsaid pedgekknqv mihgiepmle tplqwlsehl
sypdnflhis iipqptd

The invention provides for use of chimeric or hybrid polypeptides isolated from natural sources, be synthetic, or be recombinantly generated polypeptides. Peptides and proteins can be recombinantly expressed in vitro or in vivo. The chimeric peptides and polypeptides of the invention can be made and isolated using any method known in the art. Chimeric polypeptide and peptides of the invention can also be synthesized, whole or in part, using chemical methods well known in the art. See e.g., Caruthers (1980) Nucleic Acids Res. Symp. Ser. 215-223; Horn (1980) Nucleic Acids Res. Symp. Ser. 225-232; Banga, A. K., Therapeutic Peptides and Proteins, Formulation, Processing and Delivery Systems (1995) Technomic Publishing Co., Lancaster, Pa. For example, peptide synthesis can be performed using various solid-phase techniques (see e.g., Roberge (1995) Science 269;202; Merrifield (1997) Methods Enzymol. 289:3-13) and automated synthesis may be achieved, e.g., using the ABI 431A Peptide Synthesizer (Perkin Elmer) in accordance with the instructions provided by the manufacturer.

The invention provides for use of chimeric or hybrid polypeptides that are glycosylated. The glycosylation can be added post-translationally either chemically or by cellular biosynthetic mechanisms, wherein the later incorporates the use of known glycosylation motifs, which can be native to the sequence or can be added as a peptide or added in the nucleic acid coding sequence. The glycosylation can be O-linked or N-linked.

The invention provides for use of chimeric or hybrid polypeptides in any “mimetic” and/or “peptidomimetic” form. The terms “mimetic” and “peptidomimetic” refer to a synthetic chemical compound which has substantially the same structural and/or functional characteristics of the polypeptides of the invention. The mimetic can be either entirely composed of synthetic, non-natural analogues of amino acids, or, is a chimeric molecule of partly natural peptide amino acids and partly non-natural analogs of amino acids. The mimetic can also incorporate any amount of natural amino acid conservative substitutions as long as such substitutions also do not substantially alter the mimetic's structure and/or activity. As with polypeptides of the invention which are conservative variants, routine experimentation will determine whether a mimetic (e.g., use of a mimetic) is within the scope of the invention, i.e., that its structure and/or function is not substantially altered; e.g., the chimeric polypeptide of the invention retains NADH oxidoreductase activity.

The invention provides for use of chimeric or hybrid polypeptide mimetic compositions comprising any combination of non-natural structural components. In alternative aspect, mimetic compositions of the invention include one or all of the following three structural groups: a) residue linkage groups other than the natural amide bond (“peptide bond”) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like. For example, a polypeptide of the invention can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide ester, bifunctional maleimides, N,N′-dicyclohexylacarbodiimide (DCC) or N,N′-diisopropylcarbodiimide (DIC). Linking groups that can be alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., Spatola (1983) in Chemistry and Biochemistry of Amino Acids, Peptides and Proteins, Vol. 7, pp 267-357, “Peptide Backbone Modifications,” Marcell Dekker, N.Y.).

The invention provides for use of chimeric or hybrid polypeptides characterized as a mimetic by containing all or some non-natural residues in place of naturally occurring amino acid residues. Non-natural residues are well described in the scientific and patent literature; a few exemplary non-natural compositions useful as mimetics of natural amino acid residues and guidelines are described below. Mimetics of aromatic amino acids can be generated by replacing by, e.g., D- or L-naphylalanine; D- or L-phenylglycine; D- or L-2 thieneylalanine; D- or L-1, -2, 3-, or 4-pyrencylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-(trifluoromethyl)-phenylalanine; D-p-fluoro-phenylalaninel; D- or L-p-biphenylalanine; D- or L-p-methoxy-biphenylphenylalanine; D- or L-2-indole(alkyl)ananines; and, D- or L-alkylamines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, fuanyl, pyrrolyl, and pyridyl aromatic rings.

The invention provides for use of chimeric or hybrid polypeptides comprising mimetics of acidic amino acids generated by substitution by, e.g., non-carboxylate amino acids while maintaining a negative charge; (phosphone)alanine; sulfated threonine. Carboxyl side groups (e.g., aspartyl or glutamyl) can also be selectively modified by reaction with carbodiimides (R′-N-C-N-R′) such as, e.g., 1-cyclohexyl-3(2-morpholinyl-(4-ethyl) carbodiimide or 1-ethyl-3(4-azonia-4,4-diimetholpentyl) carbodiimide. Aspartyl or glutamyl can also be converted to asparaginyl and glutaminyl residues by reaction with ammonium ions. Mimetics of basic amino acids can be generated by substitution with, e.g., (in addition to lysine and arginine) the amino acids ornithine, citrulline, or (guanidino)-acetic acid, or (guanidino)alkyl-acetic acid, where alkyl is defined above. Nitrile derivative (e.g., containing the CN-moiety in place of COOH) can be substituted for asparagine or glutamine. Asparaginyl and glutaminyl residues can be deaminated to the corresponding aspartyl or glutamyl residues. Arginine residue mimetics can be generated by reacting arginyl with, e.g., one or more conventional reagents, including, e.g., phenylglyoxal, 2,3-butanedione, 1,2-cyclo-hexanedione, or ninhydrin, in one aspect under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetaone, alpha-bromo-beta-(5-imidozoyl) propionic acid; chloroacetyl phosphate, N-alkylmaleimides, 3-nitro-2-pyridyl disulfide; methyl-2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitro-benzenesulfonic acid, O-methylisourea, 2,4, pentanedione, and transamidase-catalyzed reactions with glyoxylate. Mimetics of methionine can be generated by reaction with, e.g., methionine sulfoxide. Mimetics of proline include, e.g., pipecolic acid, thiazolidine carboxylid acid, 3- or 4-hydroxy proline, dehydroproline, 3- or 4-methylproline, or 3,3,dimethylproline. Histidine residue mimetics can be generated by reacting histidyl with, e.g., diethylprocarbonate or para-bromophenacyl bromide. Other mimetics include, e.g., those generated by hydroxylation of proline and lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

The invention provides chimeric or hybrid polypeptides as described herein, further altered by either natural processes, such as post-translational processing (e.g., phosphorylation, acylation, etc), or by chemical modification techniques, and the resulting modified polypeptides. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also a given polypeptide may have many types of modifications. Modifications include acetylation, acylation, ADP-ribosylation, amidation, covalent attachment of flavin, covalent attachment of a home moiety, covalent attachment of a nucleotide or nucleotide derivative, covalent attachment of a lipid or lipid derivative, covalent attachment of a phosphatidylinositol, cross-linking cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristolyation, oxidation, pegylation, proteolytic processing, phosphorylation, prenylation, racemization, selenoylation, sulfation, and transfer-RNA mediated addition of amino acids to protein such as arginylation. See, e.g., Creighton, T. E. Proteins—Structure and Molecular Properties 2nd Ed., W.H. Freeman and Company, New York (1993); Posttranslational Covalent Modification of Proteins, B.C. Johnson, Ed., Academic Press, New York, pp. 1-12 (1983).

The invention provides chimeric or hybrid polypeptides made by solid-phase chemical peptide synthesis methods. For example, assembly of a polypeptides or peptides of the invention can be carried out on a solid support using an Applied Biosystems, Inc. Model 431A™ automated peptide synthesizer. Such equipment provides ready access to the polypeptides or peptides of the invention, either by direct synthesis or by synthesis of a series of fragments that can be coupled using other known techniques.

The invention provides chimeric or hybrid polypeptides lacking a signal peptide or comprising a heterologous signal peptide.

### Pharmaceutical Compositions and Formulations

The invention provides compositions, including pharmaceutical compositions and formulations, and methods, comprising use of cell-permeable isolated, recombinant or synthetic proteins to modulate autophagy, including a Tat-Atg5K130R (inhibitor of autophagy) and a Tat-Beclin 1 (stimulant or activator of autophagy), and nucleic acids expressing them and methods for making and using them, e.g., to treat conditions and disorders responsive to autophagy modulation (e.g., where autophagy is dysregulated), including neurodegeneration, cystic fibrosis, cancer, heart failure, diabetes, obesity, sarcopenia, aging, ischemia/reperfusion, inflammatory disorders including Crohns, ulcerative colitis, biliary cirrhosis, lysosomal storage diseases, infectious diseases associated with intracellular pathogens including viruses, bacteria, and parasites such as Trypanosomes and malaria.

In one aspect, the autophagy-modulating composition is a nucleic acid, including a vector, recombinant virus, and the like; and a recombinant hybrid (chimeric) protein is expressed in a cell in vitro, ex vivo and/or in vivo.

In alternative embodiments, in practicing use of the pharmaceutical compositions and methods of this invention, compounds that induce or upregulate hybrid (chimeric) nucleic acid and/or hybrid (chimeric) protein expression in a cell, tissue or organ are administered. For example, compounds that can be administered in practicing use of the pharmaceutical compositions and methods of this invention can comprise: an interleukin, a cytokine and/or a paracrine factor involved in survival and/or proliferative signaling; an up-regulator of AKT serine/threonine kinase; insulin-like growth factor-1 -(IGF-1); insulin; leukemia inhibitory factor (LIF); granulocyte-macrophage colony-stimulating factor (GM-CSF); or epidermal growth factor (EGF). Okadaic acid and SV40 small T antigen inhibit or block negative regulation of PIM-1 by protein phosphatase 2A, and can thus be used to increase PIM-1 levels. See Maj, et al., Oncogene 26(35):5145-53 (2007).

In alternative embodiments, the hybrid (chimeric) protein-expressing nucleic acids or hybrid (chimeric) protein compositions of the invention are formulated with a pharmaceutically acceptable carrier. In alternative embodiments, the pharmaceutical compositions of the invention can be administered parenterally, topically, orally or by local administration, such as by aerosol or transdermally. The pharmaceutical compositions can be formulated in any way and can be administered in a variety of unit dosage forms depending upon the condition or disease and the degree of illness, the general medical condition of each patient, the resulting preferred method of administration and the like. Details on techniques for formulations and administration are well described in the scientific and patent literature, see, e.g., the latest edition of Remington's Pharmaceutical Sciences, Maack Publishing Co. Easton Pa. (“Remington's”).

Therapeutic agents of the invention can be administered alone or as a component of a pharmaceutical formulation. The compounds may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the invention include those suitable for systemic administration, direct local vascular or cardiac administration, or alternatively oral/nasal, topical, parenteral, rectal, and/or intravaginal administration. The formulations may conveniently be presented in unit dosage form and may be prepared by any method well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect.

Pharmaceutical formulations of this invention may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

Pharmaceutical formulations for oral administration can be formulated using pharmaceutically acceptable carriers well known in the art in appropriate and suitable dosages. Such carriers enable the pharmaceuticals to be formulated in unit dosage forms as tablets, pills, powder, dragees, capsules, liquids, lozenges, gels, syrups, slurries, suspensions, etc., suitable for ingestion by the patient. Pharmaceutical preparations for oral use can be formulated as a solid excipients, optionally grinding a resulting mixture, and processing the mixture of granules, after adding suitable additional compounds, if desired, to obtain tablets or dragee cores. Suitable solid excipients are carbohydrate or protein fillers include, e.g., sugars, including lactose, sucrose, mannitol, or sorbitol; starch from corn, wheat, rice, potato, or other plants; cellulose such as methyl cellulose, hydroxypropylmethyl-cellulose, or sodium carboxy-methylcelluloe; and gums including arabic and tragacanth; and proteins, e.g., gelatin and collagen. Disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the invention can also be used orally using, e.g., push-fit capsules made of gelatin, as well as soft, sealed capsules made of gelatin and a coating such as glycerol or sorbitol. Push-fit capsules can contain active agents mixed with a filler or binders such as lactose or starches, lubricants such as tale or magnesium stearate, and, optionally, stabilizers. In soft capsules, the active agents can be dissolved or suspended in suitable liquids, such as fatty oils, liquid paraffin, or liquid polyethylene glycol with or without stabilizers.

Aqueous suspensions can contain an active agent (e.g., a chimeric polypeptide or peptidomimetic of the invention) in admixture with excipients suitable for the manufacture of aqueous suspensions. Such excipients include a suspending agent, such as sodium carboxymethylcellulose, methylcellulose, hydroxypropylmethylcellulose, sodium alginate, polyvinylpyrrolidone, gum tragacanth and gum acacia, and dispersin or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Oil-based pharmaceuticals can be used to deliver hybrid (chimeric) protein-expressing nucleic acids or hybrid (chimeric) protein compositions of the invention. Oil-based suspensions can be formulated by suspending an active agent in a vegetable oil, such as arachis oil, olive oil, sesame oil or coconut oil, or in a mineral oil such as liquid paraffin; or a mixture of these. See e.g., U.S. Pat. No. 5,716,928 describing using essential oils or essential oil components for increasing bioavailability and reducing inter- and intra-individual variability of orally administered hydrophobic pharmaceutical compounds (see also U.S. Pat. No. 5,858,401). The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto (1997) J. Pharmacol. Exp. Ther. 281:93-102. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

In practicing this invention, the pharmaceutical compounds can also be administered by in intranasal, intraocular and intravaginal routes including suppositories, insufflation, powders and aerosol formulations (for examples of steroid inhalents, see Rohatagi (1995) J. Clin. Pharmacol. 35:1187-1193; Tjwa (1995) Ann. Allergy Asthma Immunol. 75:107-111). Suppositories formulations can be prepared by mixing the drug with a suitable non-irritating excipient which is solid at ordinary temperatures but liquid at body temperatures and will therefore melt in the body to release the drug. Such materials are cocoa butter and polyethylene glycols.

In practicing this invention, the pharmaceutical compounds can be delivered by transdermally, by a topical route, formulated as applicator sticks, solutions, suspensions, emulsion, gels, creams, ointments, pastes, jellies, paints, powders, and aerosols.

In practicing this invention, the pharmaceutical compounds can also be delivered as microspheres for slow release in the body. For example, microspheres can be administered via intradermal injection of drug which slowly release subcutaneously; see Rao (1995) J. Biomater Sci. Polym. Ed. 7:623-645; as biodegradable and injectable gel formulations, see, e.g., Gao (1995) Pharm. Res. 12:863 (1995); or, as microspheres for oral administration, see, e.g., Eyles (1997) J. Pharm. Pharmacol. 49:669-674.

In practicing this invention, the pharmaceutical compounds can be parenterally administered, such as by intravenous (IV) administration or administration into a body cavity or lumen of the heart. Use of catheters that temporarily block flow of blood from the heart while incubating the stem cells or a viral construct in heart tissue can be used, as well as recirculation systems of well-known type that isolate the circulation in all or a part of the heart to increase the dwell time of an introduced agent (e.g., stem cell, construct, naked DNA, PIM protein, viral or other vector) in the heart. These formulations can comprise a solution of active agent dissolved in a pharmaceutically acceptable carrier. Acceptable vehicles and solvents that can be employed are water and Ringer's solution, an isotonic sodium chloride. In addition, sterile fixed oils can be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of active agent in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated using those suitable dispersing or wetting agents and suspending agents. The sterile injectable preparation can also be a suspension in a nontoxic parenterally-acceptable diluent or solvent, such as a solution of 1,3-butanediol. The administration can be by bolus or continuous infusion (e.g., substantially uninterrupted introduction into a blood vessel for a specified period of time).

The pharmaceutical compounds and formulations of the invention can be lyophilized. The invention provides a stable lyophilized formulation comprising a composition of the invention, which can be made by lyophilizing a solution comprising a pharmaceutical of the invention and a bulking agent, e.g., mannitol, trehalose, raffinose, and sucrose or mixtures thereof. A process for preparing a stable lyophilized formulation can include lyophilizing a solution about 2.5 mg/mL protein, about 15 mg/mL sucrose, about 19 mg/mL NaCl, and a sodium citrate buffer having a pH greater than 5.5 but less than 6.5. See, e.g., U.S. patent application No. 20040028670.

The compositions and formulations of the invention can be delivered by the use of liposomes (see also discussion, below). By using liposomes, particularly where the liposome surface carries ligands specific for target cells, or are otherwise preferentially directed to a specific organ, one can focus the delivery of the active agent into target cells of the heart or other part of the circulatory system in vivo. See, e.g., U.S. Pat. Nos. 6,063,400; 6,007,839; Al-Muhammed (1996) J. Microencapsul. 13:293-306; Chonn (1995) Curr. Opin. Biotechnol. 6:698-708; Ostro (1989) Am. J. Hosp. Pharm. 46:1576-1587.

The formulations of the invention can be administered for prophylactic and/or therapeutic treatments. In therapeutic applications, compositions are administered to a subject already suffering from a condition, infection or disease in an amount sufficient to cure, alleviate or partially arrest the clinical manifestations of the condition, infection or disease and its complications (a “therapeutically effective amount”). For example, in alternative embodiments, pharmaceutical compositions of the invention are administered in an amount sufficient to treat, prevent and/or ameliorate a condition or disorder responsive to autophagy modulation (e.g., where autophagy is dysregulated), including neurodegeneration, cystic fibrosis, cancer, heart failure, diabetes, obesity, sarcopenia, aging, ischemia/reperfusion, inflammatory disorders including Crohns, ulcerative colitis, biliary cirrhosis, lysosomal storage diseases, infectious diseases associated with intracellular pathogens including viruses, bacteria, and parasites such as Trypanosomes and malaria.

The amount of pharmaceutical composition adequate to accomplish this can be a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age and the like. In calculating the dosage regimen for a patient, the mode of administration also is taken into consideration.

The dosage regimen also takes into consideration pharmacokinetics parameters well known in the art, i.e., the active agents' rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). The state of the art allows the clinician to determine the dosage regimen for each individual patient, active agent and disease or condition treated. Guidelines provided for similar compositions used as pharmaceuticals can be used as guidance to determine the dosage regiment, i.e., dose schedule and dosage levels, administered practicing the methods of the invention are correct and appropriate.

Single or multiple administrations of formulations can be given depending on the dosage and frequency as required and tolerated by the patient. The formulations should provide a sufficient quantity of active agent to effectively treat, prevent or ameliorate a conditions, diseases or symptoms as described herein. Methods for preparing parenterally or non-parenterally administrable formulations are know or apparent to those skilled in the art and are described in more detail in such publications as Remington's.

The methods of the invention can further comprise co-administration with other drugs or pharmaceuticals, e.g., compositions. For example, the methods and/or compositions and formulations of the invention can be co-formulated with and/or co-administered with antibiotics (e.g., antibacterial or bacteriostatic peptides or proteins), particularly those effective against gram negative bacteria, fluids, cytokines, immunoregulatory agents, anti-inflammatory agents, complement activating agents, such as peptides or proteins comprising collagen-like domains or fibrinogen-like domains (e.g., a ficolin), carbohydrate-binding domains, and the like and combinations thereof.

### In Vivo Nucleic Acid Delivery—Gene Therapy Delivery

In alternative embodiments, the hybrid (chimeric) proteins used to practice this invention are delivered to a cell, tissue or organ in vitro, in situ, ex vivo, and/or in vivo, via protein-expressing nucleic acids. Hybrid (chimeric) proteins used to practice this invention can be delivered for ex vivo or in vivo gene therapy to deliver a protein-encoding nucleic acid. In one aspect, hybrid (chimeric) protein-expressing nucleic acid compositions of the invention include non-reproducing viral constructs expressing high levels of hybrid (chimeric) protein, which can be delivered ex vivo or for in vivo gene therapy.

In alternative embodiments, the hybrid (chimeric) protein-expressing nucleic acid compositions of the invention can be delivered to and expressed in a variety of cells, tissues, organs to either stimulate or inhibit the process of autophagy: e.g., in one embodiment, to inhibit autophagy, such as Atg5K130R (Tat-Atg5^{K130R}), or a Beclin 1 to stimulate or activate autophagy.

In alternative embodiments, the invention provides use of hybrid (chimeric) protein-expressing nucleic acid for a clinical therapy for treatment of a number of organs, cells or tissues. For example, hybrid (chimeric) protein-expressing nucleic acid delivery vehicles, e.g., expression constructs, such as vectors or recombinant viruses, can be injected directly into the organ (e.g., a brain, heart, etc.) to protect it from immediate injury, or as a therapeutic or a prophylactic agent. In alternative embodiments, expression of the hybrid (chimeric) protein can be then activated through an oral prescription drug (formulations for which are discussed above).

In one embodiment vectors used to practice this invention, e.g., to generate a hybrid (chimeric) protein-expressing cell, are bicistronic. In one embodiment, a MND (or, mycloproliferative sarcoma virus LTR-negative control region deleted) promoter is used to drive hybrid (chimeric) protein expression. In one embodiment, a reporter is also used, e.g., an enhanced green florescent protein (eGFP) reporter, which can be driven off a viral internal ribosomal entry site (vIRES). In alternative embodiments, all constructs are third generation self-inactivating (SIN) lentiviral vectors and incorporate several elements to ensure long-term expression of the transgene. For example, a MND promoter allows for high expression of the transgene, while the LTR allows for long-term expression after repeated passage. In alternative embodiments, the vectors also include (IFN)-β-scaffold attachment region (SAR) element; SAR elements have been shown to be important in keeping the vector transcriptionally active by inhibiting methylation and protecting the transgene from being silenced.

In alternative embodiments, as a secondary course of therapy, hybrid (chimeric) protein-expressing nucleic acid delivery vehicles, e.g., expression constructs, such as vectors or recombinant viruses, can be used to enhance hybrid (chimeric) protein-expressing expression in vivo. In alternative embodiments, liposomes are used to deliver hybrid (chimeric) protein-expressing nucleic acids.

In alternative embodiments hybrid (chimeric) protein-expressing nucleic acids are activated to express through addition of the drug to culture media. After a number of days in culture, the expression of hybrid (chimeric) protein can be then turned off through removal of the drug; and, in one aspect, the increased number of cells produced in culture are reintroduced into the damaged area contributing to an enhanced repair process.

In alternative embodiments the invention uses any non-viral delivery or non-viral vector systems are known in the art, e.g., including lipid mediated transfection, liposomes, immunoliposomes, lipofectin, cationic facial amphiphiles (CFAs) and combinations thereof.

In alternative embodiments, expression vehicles, e.g., vectors or recombinant viruses, used to practice the invention are injected directly into the heart muscle. In one aspect, the hybrid (chimeric) protein encoding nucleic acid is administered to the individual by direct injection. Thus, in one embodiment, the invention provides sterile injectable formulations comprising expression vehicles, e.g., vectors or recombinant viruses, used to practice the invention.

In alternative embodiments, it may be appropriate to administer multiple applications and employ multiple routes, e.g., directly into the heart muscle and intravenously, to ensure sufficient exposure of target cells (e.g., myocytes or stem cells) to the expression construct. Multiple applications of the expression construct may also be required to achieve the desired effect.

In alternative embodiments, the invention provides for ex vivo modification of cells, e.g., a stem cell, or a cell of any origin (e.g., a pluripotent cell) to enhance hybrid (chimeric) protein expression, followed by administration of the stem cells to a human or other mammalian host, or to any vertebrate. The cells may be directly or locally administered, for example, into a tissue or organ, or by systemic administration. The stem cells may be autologous stem cells or heterologous stem cells. They may be derived from embryonic sources or from infant or adult organisms. Hybrid (chimeric) protein-encoding nucleic acids in cells may advantageously be under inducible expression control.

In alternative embodiments, a “suicide sequence” is incorporated into a chimeric nucleic acid of the invention. In alternative embodiments, one or more “suicide sequence” are also administered, either separately or in conjunction with a nucleic acid construct of this invention, e.g., incorporated within the same nucleic acid construct (such as a vector, recombinant virus, and the like. See, e.g., Marktel S, et al., Immunologic potential of donor lymphocytes expressing a suicide gene for early immune reconstitution after hematopoietic T-cell-depleted stem cell transplantation. Blood 101:1290-1298(2003). Suicide sequences used to practice this invention can be of known type, e.g., sequences to induce apoptosis or otherwise cause cell death, e.g., in one aspect, to induce apoptosis or otherwise cause cell death upon administration of an exogenous trigger compound or exposure to another type of trigger, including but not limited to light or other electromagnetic radiation exposure.

In alternative embodiments, a hybrid (chimeric) protein-encoding nucleic acid-comprising expression construct or vehicle of the invention is formulated at an effective amount of ranging from about 0.05 to 500 μg/kg, or 0.5 to 50 μg/kg body weight, and can be administered in a single dose or in divided doses. In alternative embodiments the amount of a hybrid (chimeric) protein-encoding nucleic acid of the invention, or other the active ingredient (e.g., an inducing or upregulating agent) actually administered is determined in light of various relevant factors including the condition to be treated, the age and weight of the individual patient, and the severity of the patient's symptom; and, therefore, the above dose should not be intended to limit the scope of the invention in any way.

In alternative embodiments, a hybrid (chimeric) protein-encoding nucleic acid-comprising expression construct or vehicle of the invention is formulated at a titer of about at least 10¹⁰, 10¹¹, 10¹², 10¹³, 10¹⁴, 10¹⁵, 10¹⁶, or 10¹⁷ physical particles per milliliter. In one aspect, the PIM-1 encoding nucleic acid is administered in about 10, 20, 30, 40, 50, 60, 70, 80, 90, 100, 110, 120, 130, 140 or 150 or more microliter (μl) injections. Doses and dosage regimens can be determined by conventional range-finding techniques known to those of ordinary skill in the art. In alternative embodiments, about 10⁶, 10⁷, 10⁸, 10⁹, 10¹⁰, 10¹¹, 10¹², 10¹³, 10¹⁴, 10¹⁵, 10¹⁶ or 10¹⁷ viral (e.g., lentiviral) particles are delivered to the individual (e.g., a human patient) in one or multiple doses.

In other embodiments, an intra-tissue (e.g., an intracardiac) single administration (e.g., a single dose) comprises from about 0.1 μl to 1.0 μl, 10 μl or to about 100 μl of a pharmaceutical composition of the invention. In alternative embodiments, dosage ranges from about 0.5 ng or 1.0 ng to about 10 μg, 100 μg to 1000 μg of PIM-1 expressing nucleic acid is administered (either the amount in an expression construct, or as in one embodiment, naked DNA is injected). Any necessary variations in dosages and routes of administration can be determined by the ordinary skilled artisan using routine techniques known in the art.

In one embodiment, a hybrid (chimeric) protein-expressing necleic acid is delivered in vivo directly to a heart using a viral stock in the form of an injectable preparation containing pharmaceutically acceptable carrier such as saline. The final titer of the vector in the injectable preparation can be in the range of between about 10⁸ to 10¹⁴, or between about 10¹⁰ to 10¹², viral particles; these ranges can be effective for gene transfer.

In alternative embodiments, hybrid (chimeric) protein-expressing nucleic acids (e.g., vector, transgene) constructs are delivered to a tissue or organ (e.g., a myocardium by direct (e.g., by intracoronary) injection, e.g., using a standard percutaneous catheter based methods under fluoroscopic guidance. In alternative embodiments, hybrid (chimeric) protein-expressing nucleic acids (e.g., vector, transgene) constructs are delivered to organs and tissues, e.g., the heart, directly into both coronary and/or peripheral arteries, e.g., using a lipid-mediated gene transfer.

In alternative embodiments, including direct organ or tissue injection (e.g., an intracoronary injection, or directly into both coronary and/or peripheral arteries), can be at an amount sufficient for the hybrid (chimeric) protein-expressing nucleic acids (e.g., vector, transgene) to be expressed to a degree which allows for sufficiently effective; e.g., the amount of the hybrid (chimeric) protein-expressing nucleic acid (e.g., vector, transgene) injected can be in the range of between about 10⁸ to 10¹⁴, or between about 10¹⁰ to 10¹², viral particles.

In alternative embodiments the injection can be made deeply (e.g., such as 1 cm within the arterial lumen) into the lumen of the coronary arteries, and can be made in both coronary arteries, as the growth of collateral blood vessels is highly variable within individual patients. By injecting the material directly into the lumen of the coronary artery by coronary catheters, it is possible to target the protein-expressing nucleic acid (e.g., vector, transgene) effectively and to minimize loss of recombinant vectors to the proximal aorta during injection. Any variety of coronary catheter, or Stack perfusion catheters, and the like can be used. See, e.g., U.S. Patent App. Pub. No. 20040132190.

In alternative embodiments, the invention combines a therapeutic nucleic acid with a genetic “sensor”, e.g., that recognizes and responds to the oxygen deprivation that follows the reduced blood flow, or ischemia, from coronary artery disease and heart attack. As soon as the oxygen declines, the sensor turns on the therapeutic gene, thereby protecting the heart. In addition to its potential for patients with heart disease, the aspect of this invention is useful for any condition in which circulatory system tissues are susceptible to loss of blood supply, including stroke, shock, trauma and sepsis.

In alternative embodiments, the invention provides a retroviral, e.g., a lentiviral, vector capable of delivering a nucleotide sequence encoding a hybrid (chimeric) protein of this invention in vitro, ex vivo and/or in vivo. In alternative embodiments, a lentiviral vector used to practice this invention is a “minimal” lentiviral production system lacking one or more viral accessory (or auxiliary) gene. Exemplary lentiviral vectors for use in the invention can have enhanced safety profiles in that they are replication defective and self-activating (SIN) lentiviral vectors. Lentiviral vectors and production systems that can be used to practice this invention include e.g., those described in U.S. Pat. Nos. (USPNs) 6,277,633; 6,312,682; 6,312,683; 6,521,457; 6,669,936; 6,924,123; 7,056,699; and U.S. Pat. No. 7,198,784; and combination of these are exemplary vectors that can be employed in the practice of the invention. In an alternative embodiment, non-integrating lentiviral vectors can be employed in the practice of the invention. For example, non-integrating lentiviral vectors and production systems that can be employed in the practice of the invention include those described in U.S. Pat. No. 6,808,923.

The expression vehicle can be designed from any vehicle known in the art, e.g., a recombinant adeno-associated viral vector as described, e.g., in U.S. Pat. App. Pub. No. 20020194630, Manning, et al.; or a lentiviral gene therapy vector, e.g., as described by e.g., Dull, et al. (1998) J. Virol. 72:8463-8471; or a viral vector particle, e.g., a modified retrovirus having a modified proviral RNA genome, as described, e.g., in U.S. Pat. App. Pub. No. 20030003582; or an adeno-associated viral vector as described e.g., in U.S. Pat. No. 6,943,153, describing recombinant adeno-associated viral vectors for use in the eye; or a retroviral or a lentiviral vector as described in U.S. Pat. Nos. 7,198,950; 7,160,727; 7,122,181 (describing using a retrovirus to inhibit intraocular neovascularization in an individual having an age-related macular degeneration); or U.S. Pat. No. 6,555,107.

Any viral vector can be used to practice this invention, and the concept of using viral vectors for gene therapy is well known; see e.g., Verma and Somia (1997) Nature 389:239-242; and Coffin et al (“Retroviruses” 1997 Cold Spring Harbour Laboratory Press Eds; J M Coffin, S M Hughes, H E Varmus pp 758-763) having a detailed list of retroviruses. Any lentiviruses belonging to the retrovirus family can be used for infecting both dividing and non-dividing cells with a PIM-1-encoding nucleic acid, see e.g., Lewis et al (1992) EMBO J. 3053-3058.

Viruses from lentivirus groups from “primate” and/or “non-primate” can be used; e.g., any primate lentivirus can be used, including the human immunodeficiency virus (HIV), the causative agent of human acquired immunodeficiency syndrome (AIDS), and the simian immunodeficiency virus (SIV); or a non-primate lentiviral group member, e.g., including “slow viruses” such as a visna/maedi virus (VMV), as well as the related caprine arthritis-encephalitis virus (CAEV), equine infectious anemia virus EIAV) and/or a feline immunodeficiency virus (FIV) or a bovine immunodeficiency virus (BIV).

In alternative embodiments, lentiviral vectors used to practice this invention are pseudotyped lentiviral vectors. In one aspect, pseudotyping used to practice this invention incorporates in at least a part of, or substituting a part of, or replacing all or, an env gene of a viral genome with a heterologous env gene, for example an env gene from another virus. In alternative embodiments, the lentiviral vector of the invention is pseudotyped with VSV-G. In an alternative embodiment, the lentiviral vector of the invention is pseudotyped with Rabies-G.

Lentiviral vectors used to practice this invention may be codon optimized for enhanced safety purposes. Different cells differ in this usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. Many viruses, including HIV and other lentiviruses, use a large number of rare codons and by changing these to correspond to commonly used mammalian codons, increased expression of the packaging components in mammalian producer cells can be achieved. Codon usage tables are known in the art for mammalian cells, as well as for a variety of other organisms. Codon optimization has a number of other advantages. By virtue of alterations in their sequences, the nucleotide sequences encoding the packaging components of the viral particles required for assembly of viral particles in the producer cell/packaging cells have RNA instability sequences (INS) eliminated from them. At the same time, the amino acid sequence coding sequence for the packaging components is retained so that the viral components encoded by the sequences remain the same, or at least sufficiently similar that the function of the packaging components is not compromised. Codon optimization also overcomes the Rev/RRE requirement for export, rendering optimized sequences Rev independent. Codon optimization also reduces homologous recombination between different constructs within the vector system (for example between the regions of overlap in the gag-pol and env open reading frames). The overall effect of codon optimization is therefore a notable increase in viral titer and improved safety. The strategy for codon optimized gag-pol sequences can be used in relation to any retrovirus.

Vectors, recombinant viruses, and other expression systems used to practice this invention can comprise any nucleic acid which can infect, transfect, transiently or permanently transduce a cell. In one aspect, a vector used to practice this invention can be a naked nucleic acid, or a nucleic acid complexed with protein or lipid. In one aspect, a vector used to practice this invention comprises viral or bacterial nucleic acids and/or proteins, and/or membranes (e.g., a cell membrane, a viral lipid envelope, etc.). In one aspect, expression systems used to practice this invention comprise replicons (e.g., RNA replicons, bacteriophages) to which fragments of DNA may be attached and become replicated. In one aspect, expression systems used to practice this invention include, but are not limited to RNA, autonomous self-replicating circular or linear DNA or RNA (e.g., plasmids, viruses, and the like, see, e.g., U.S. Pat. No. 5,217,879), and include both the expression and non-expression plasmids.

In one aspect, a recombinant microorganism or cell culture used to practice this invention can comprise “expression vector” including both (or either) extra-chromosomal circular and/or linear nucleic acid (DNA or RNA) that has been incorporated into the host chromosome(s). In one aspect, where a vector is being maintained by a host cell, the vector may either be stably replicated by the cells during mitosis as an autonomous structure, or is incorporated with the host's genome.

In one aspect, an expression system used to practice this invention can comprise any plasmid, which are commercially available, publicly available on an unrestricted basis, or can be constructed from available plasmids in accord with published procedures. Plasmids that can be used to practice this invention are well known in the art.

In alternative aspects, a vector used to make or practice the invention can be chosen from any number of suitable vectors known to those skilled in the art, including cosmids, YACs (Yeast Artificial Chromosomes), mega YACS, BACs (Bacterial Artificial Chromosomes), PACs (P1 Artificial Chromosome), MACs (Mammalian Artificial Chromosomes), a whole chromosome, or a small whole genome. The vector also can be in the form of a plasmid, a viral particle, or a phage. Other vectors include chromosomal, non-chromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by, e.g., Sambrook. Bacterial vectors which can be used include commercially available plasmids comprising genetic elements of known cloning vectors.

### Nanoparticles and Liposomes

The invention also provides nanoparticles and liposomal membranes comprising the hybrid (chimeric) protein-expressing compounds of this invention which target specific molecules, including biologic molecules, such as polypeptide, including cardiac or vascular or stem cell surface polypeptides, including heart cell (e.g., myocyte) cell surface polypeptides. In alternative embodiments, the invention provides nanoparticles and liposomal membranes targeting diseased and/or injured heart cells, or stem cells, such as any pluripotent cell.

In alternative embodiments, the invention provides nanoparticles and liposomal membranes comprising (in addition to comprising compounds of this invention) molecules, e.g., peptides or antibodies, that selectively target diseased and/or injured cells, organs or tissues, e.g., brain or heart cells, or stem cells. In alternative embodiments, the invention provides nanoparticles and liposomal membranes using interleukin receptors and/or other receptors to target receptors on cells, e.g., diseased and/or injured cells, organs or tissues, e.g., brain or heart cells, or stem cells. See, e.g., U.S. patent application publication No. 20060239968.

Thus, in one aspect, the compositions of the invention are specifically targeted to cells, organs or tissues, e.g., brain or stem cells or heart cells, such as myocytes.

The invention also provides nanocells to allow the sequential delivery of two different therapeutic agents with different modes of action or different pharmacokinetics, at least one of which comprises a hybrid (chimeric) protein of this invention. A nanocell is formed by encapsulating a nanocore with a first agent inside a lipid vesicle containing a second agent; see, e.g., Sengupta, et al., U.S. Pat. Pub. No. 20050266067. The agent in the outer lipid compartment is released first and may exert its effect before the agent in the nanocore is released. The nanocell delivery system may be formulated in any pharmaceutical composition for delivery to patients suffering from any disease or condition as described herein, e.g., neurodegeneration, cystic fibrosis, cancer, heart failure, diabetes, obesity, sarcopenia, aging, ischemia/reperfusion, inflammatory disorders including Crohns, ulcerative colitis, biliary cirrhosis, lysosomal storage diseases, infectious diseases associated with intracellular pathogens including viruses, bacteria, and parasites such as Trypanosomes and malaria, or congestive heart failure or heart attack (myocardial infarction). For example, an antibody and/or angiogenic agent can be contained in the outer lipid vesicle of the nanocell, and a composition of this invention is loaded into the nanocore. This arrangement allows the antibody and/or angiogenic agent to be released first and delivered to the diseased or injured tissue.

The invention also provides multilayered liposome comprising compounds of this invention, e.g., for transdermal absorption, e.g., as described in Park, et al., U.S. Pat. Pub. No. 20070082042. The multilayered liposomes can be prepared using a mixture of oil-phase components comprising squalane, sterols, ceramides, neutral lipids or oils, fatty acids and lecithins, to about 200 to 5000 nm in particle size, to entrap a composition of this invention.

A multilayered liposome of the invention may further include an antiseptic, an antioxidant, a stabilizer, a thickener, and the like to improve stability. Synthetic and natural antiseptics can be used, e.g., in an amount of 0.01% to 20%. Antioxidants can be used, e.g., BHT, erysorbate, tocopherol, astaxanthin, vegetable flavonoid, and derivatives thereof, or a plant-derived antioxidizing substance. A stabilizer can be used to stabilize liposome structure, e.g., polyols and sugars. Exemplary polyols include butylene glycol, polyethylene glycol, propylene glycol, dipropylene glycol and ethyl carbitol; examples of sugars are trehalose, sucrose, mannitol, sorbitol and chitosan, or a monosacchardie or an oligosaccharide, or a high molecular weight starch. A thickener can be used for improving the dispersion stability of constructed liposomes in water, e.g., a natural thickener or an acrylamide, or a synthetic polymeric thickener. Exemplary thickeners include natural polymers, such as acacia gum, xanthan gum, gellan gum, locust bean gum and starch, cellulose derivatives, such as hydroxy ethylcellulose, hydroxypropyl cellulose and carboxymethyl cellulose, synthetic polymers, such as plyacrylic acid, polyacrylamide or polyvinylpyrollidone and polyvinylalcohol, and copolymers thereof or cross-linked materials.

Liposomes can be made using any method, e.g., as described in Park, et al., U.S. Pat. Pub. No. 20070042031, including method of producing a liposome by encapsulating a therapeutic product comprising providing an aqueous solution in a first reservoir; providing an organic lipid solution in a second reservoir, wherein one of the aqueous solution and the organic lipid solution includes a therapeutic product; mixing the aqueous solution with said organic lipid solution in a first mixing region to produce a liposome solution, wherein the organic lipid solution mixes with said aqueous solution so as to substantially instantaneously produce a liposome encapsulating the therapeutic product; and immediately thereafter mixing the liposome solution with a buffer solution to produce a diluted liposome solution.

The invention also provides nanoparticles comprising compounds of this invention to deliver a composition of the invention as a drug-containing nanoparticles (e.g., a secondary nanoparticle), as described, e.g., in U.S. Pat. Pub. No. 20070077286. In one embodiment, the invention provides nanoparticles comprising a fat-soluble drug of this invention or a fat-solubilized water-soluble drug to act with a bivalent or trivalent metal salt.

### Kits

The invention provides kits comprising a chimeric (fusion) polypeptide of the invention (e.g., a recombinant or synthetic chimeric molecule), a chimeric (fusion) polynucleotide (e.g., a recombinant or synthetic chimeric molecule) of the invention, or a pharmaceutical composition of the invention, including instructions on practicing the methods of the invention, e.g., directions as to indications, dosages, patient populations, routes and methods of administration.

The invention will be further described with reference to the following examples; however, it is to be understood that the invention is not limited to such examples.

### EXAMPLES

### Example 1

### Autophagy is Required for Preconditioning By the Adenosine A1 Receptor-Selective Agonist CCPA

The following example describes making and using exemplary polypeptides of this invention; and demonstrates their efficacy.

We have shown that the cellular process of macroautophagy plays a protective role in HL-1 cardiomyocytes subjected to simulated ischemia/reperfusion (SI/R)¹. Since the nucleotide adenosine has been shown to mimic both early and late phase ischemic preconditioning, a potent cardioprotective phenomenon, the purpose of this study was to determine the effect of adenosine on autophagosome formation. Autophagy is a highly regulated intracellular degradation process by which cells remove cytosolic long-lived proteins and damaged organelles, and can be monitored by imaging the incorporation of microtubule-associated light chain 3 (LC3) fused to a fluorescent protein (GFP or mCherry) into nascent autophagosomes. We investigated the effect of adenosine receptor agonists on autophagy and cell survival following sI/R in GFP-LC3 infected HL-1 cells and neonatal rat dardiomyocytes. The A₁ adenosine receptor agonist 2-chloro-N(6)-cyclopentyladenosine (CCPA)(100 nM) caused an increase in the number of autophagosomes within 10 min of treatment; the effect persisted for at least 300 min. A significant inhibition of autophagy and loss of protection against sI/R measured by release of lactate dehydrogenase (LDH), was demonostrated in CCPA-pretreated cells treated with an A₁ receptor antagonist, a phospholipase C inhibitor, or an intracellular (Ca(+2) chelator. To determine whether autophagy was required for the protective effect of CCPA, autophagy was blocked with a dominant negative inhibitor (Atg5^{K130R}) delivered by transient transfection (in HL-1 cells) or protein transduction (in adult rat cardiomyocytes), CCPA attenuated LDH release after sI/R, but protection was lost when autophagy was blocked. To assess autophagy in vivo, transgenic mice expressing the read fluorescent autophagy marker mCherry-LC3 under the control of the alpha myosin heavy chain promoter were treated with CCPA 1 mg/kg i.p. Fluorescence microscopy of cryosections taken from the left ventricle 30 min after CCPA injection revealed a large increase in the number of mCherry-LC3-labeled structures, indicating the induction of autophagy by CCPA in vivo. Taken together, these results indicate that autophagy plays an important role in mediating the cardioprotective effects conferred by adenosine pretreatment.

Since the end-effector(s) of adenosine-mediated protection is unknown, the purpose of this study was to test the hypothesis that adenosine-mediated cardioprotection requires activation of autophagy, and that autophagy is necessary and sufficient for achieving cardioprotection. We subjected a HL-1 myocyte cell line to simulated I/R and treated mCherry-LC3 transgenic mice with 2-chloro-N(6)-cyclopentyladenoise (CCPA), a selective adenosine A₁ receptor agonist.

### Experimental Procedures

### Reagents

BAPTA-AM and Bafilomycin A1 (Baf) were purchased from EMD Biosciences (San Diego, Calif.); CCPA, DPCPX and thapsigargin (TG) were purchased from Sigma (St Louis, Mo.).

Cell Culture

Cells of the murine atrial-derived cardiac cell line HL-1¹⁶ were plated in gelatin/fibronectin-coated culture vessels and maintained in Claycomb medium¹⁶ (JRH Biosciences, Lenexa, Kans.) supplemented with 10% fetal bovine serum, 0.1 mm norepinephrine, 2 mm 1-glutamine, 100 U·mL⁻¹ penicillin, 100 U·mL⁻¹ streptomycin, and 0.25 μg·mL⁻¹ amphotericin B.

Freshly isolated adult rat cardiomyocytes were prepared from 200-250 gr male Sprague Dawley rats, following standard methods. The animals were anesthetized with sodium pentobarbital, and all animal procedures were in accordance with institutional guideline and approved by the Institutional Animal Care and Use Committee. After an injection of heparin (100 U/kg) into the hepatic vein, the heart was excised and the aorta was cannulated. The heart was perfused retrogradely with a Ca²⁺-free buffer followed by perfusion with 0.6 mg/mL collagenase (CLS 2, Worthington Biochemical Corporation, USA) and 8.3 μM CaCl₂ in perfusion buffer. After perfusion with collagenase solution for 15 min, the heart was minced in the same collagenase solution and the myocytes were filtered through a fine gauze. A stopping buffer containing 5% bovine calf serum and 12.5 μM CaCl₂ was added to the cells, followed by calcium stepwise reintroduction up to a concentration of 1 mM. The cells were centrifuged at 100 ×g for 1 min, and the pellet was washed in M199 medium (Invitrogen), containing 10 mM HEPES, 5 mM taurine, 5 mM creatine, 2 mM carnitine, 0.5% free fatty acid BSA and 100 U/mL penicillin-streptomycin. Cardiomyocytes were plated with laminin (Roche) (20 μg/mL laminin for glass, or 10 μg/mL for plastic dishes) at 5×10⁴ cells per dish. The cells were incubated in a 5% CO₂ incubator at 37° C. for 2 hr, then the medium was replaced with the same fresh medium, and the experiments were performed 24 hr later. Cell viability based on rod-shaped morphology at the outset of the experiment was routinely >90%.

Transfections, Infections, and Protein Transduction

HL-1 cells were transfected with the indicated vectors using the transfection reagent EFFECTENE™ (Qiagen, Valencia, Calif.), according to the manufacturer's instructions, achieving at least 40% transfection efficiency. For experiments aimed at determining autophagic flux, HL-1 cells ere transfected with GFP-LC3 and the indicated vector at a ratio of 1:3 μg DNA. For infections, HL-1 cells or adult rat cardiomyocytes were infected with GFP-LC3 adenovirus for two hr, washed in PBS and re-fed with the Claycomb medium or M199 medium respectively. All the experiments were performed 20 hr after infection. The dominant negative pmCherryAtg5^{K130R} was previously described¹ and has been deposited with ADDGENE™. For adult cardiomyocytes, GFP-LC3 infected cells were incubated with recombinant Tat-Atg5^{K130R} for 30 min before adding CCPA. Tat-Atg5^{K130R} was prepared by cloning Atg5^{K130R} into the pHA-TAT construct previously described¹⁷. Recombinant protein was purified as previously described^{11, 17, 18}.

High- and Low-nutrient Conditions

Cells were plated in 14-mm-diameter glass bottom microwell dishes (MatTek, Ashland, Mass.). For high-nutrient conditions, experiments were performed in fully supplemented Claycomb medium. For low-nutrient conditions, experiments were performed in modified Krebs-Henseleit buffer (MKH) (in mM: 110 NaCl, 4.7 KCl, 1.2 KH₂PO₄, 1.25 MgSO₄, 1.2 CaCl₂, 25 NaHCO₃, 15 glucose, 20 HEPES, pH 7.4) and incubation at 95% room air—5% CO₂.

Simulated Ischemia/Reperfusion (sI/R)

Cells were plated in 14-mm diameter glass bottom microwell dishes (MatTek), and ischemia was introduced by a buffer exchange to ischemia-mimetic solution (in mM: 20deoxyglucose, 125 NaCl, 8 KCl, 1.2 KH₂PO₄, 1.25 MgSO₄, 1.2 CaCl₂, 6.25 NaHCO₂, 5 sodium lactate, 20 HEPES, pH 6.6) and placing the dishes in hypoxic pouches (GasPak™ EZ, BD Biosciences) equilibrated with 95% N₂, 5% CO₂. After 2 hr of simulated ischemia, reperfusion was initiated by a buffer exchange to normoxic MKH buffer and incubation at 95% room air, 5% CO₂. Controls incubated in normoxic MKH buffer were run in parallel for each condition for periods of time that corresponded with those of the experimental groups.

Wide-field Fluorescence Microscopy

Cells were observed through a Nikon TE300™ fluorescence microscope (Nikon, Melville, N.Y.) equipped with a ×10 lens (0.3 NA, Nikon), a ×40 Plan Fluor and a ×60 Plan Apo™ objective (1.4 NA and 1.3 NA oil immersion lenses; Nikon), a Z-motor (ProScanII™, Prior Scientific, Rockland, Mass.), a cooled CCD camera (Orca-ER™, Hamamatsu, Bridgewater, N.J.) and automated excitation and emission filter wheels controlled by a LAMBDA 10-2™ (Sutter Instrument, Novato, Calif.) operated by MetaMorph 6.2r4™ (Molecular Devices Co., Downington, Pa.). Fluorescence was excited through an excitation filter for fluorescein isothiocyanate (HQ480/×40), and an emission filter (HQ535/50 m).

Determination of Autophagic Content and Flux

To analyze autophagic flux, GFP-LC3-expressing cells were subjected to the indicated experimental conditions with and without a cell-permeable lysosomal inhibitor Bafilomycin A1 (50 nm, vacuolar H⁺-ATPase inhibitor) to inhibit autophagosome-lysosome fusion¹⁹, for an interval of 3 hr. Cells were fixed with 4% formaldehyde in PBS (pH 7.4) for 15 min.

To analyze the number of GFP-LC3 puncta in population, cells were inspected at 60× magnification and classified as: (a) cells with predominantly diffuse GFP-LC3 fluorescence; or as (b) cells with numerous GFP-LC3 puncta (>30 dots/cell), representing autophagosomes. At least 200 cells were scored for each condition in three or more independent experiments.

Experiments With Preconditioning Agents

2-chloro-N(6)-cyclopentyladenosine (CCPA) at concentrations of 0.001-0.1 nM was applied to the cell cultures for 15 min following a 15 min preincubation with various inhibitors (Sigma); 8-cyclopentyl-1,3-dimentylxanthine (DPCPX, 1 μM), BAPTA-AM (25 μM), U73122 (2 μM) or thapsigargin (TG, 1 μM). The cell cultures were washed with PBS prior to the experimental treatment.

Release of LDH

Protein content and LDH activity were determined according to El-Ani et al.²⁰. Briefly, 25 μl supernatants from 35 mm dishes were transferred into wells of a 96-well plate, and the LDH activities were determined with an LDH-L kit (Sigma), according to the manufacturer. The product of the enzyme was measured spectrophotometrically at 30° C. at a wavelength of 340 nm as described previously²¹. The results were expressed relative to the control (X-fold) in the same experiment. Each experiment was done in triplicate and was repeated at least three times.

Nuclear Staining

Cells were stained immediately after sI/R with propidium iodide (5 μg/ml), which stains nuclei of cells whose plasma membranes have become permeable because of cell damage. The assay was performed according to Nieminen et al.²². For counterstaining we used Hoechst 33342 (10 μM), which stains the nuclei of all cells.

Transgenic mCherry-LC3 mice-Cardiac-specific expressing mCherry-LC3 transgenic mice were created in the FVB/N strain by pronuclear injection of murine alpha myosin heavy chain promoter driven mCherry-LC3 transgene in front of the human growth hormone poly adenylation signal²³. Mice were injected with saline or CCPA (1 mg/kg, i.p.), and 30 min later they were euthanized with pentobarbital and the hearts excised and embedded in Optimal Cutting Temperature medium for cryosectioning and fluorescence microscopy. All animal procedures were carried out in accordance with institutional guidelines and approved by the Institutional Animal Care and Use Committee.

Statistics

The probability of statistically significant differences between two experimental groups was determined by Student's t-test. Values are expressed as mean ±SEM of at least three independent experiments unless stated otherwise.

### Results

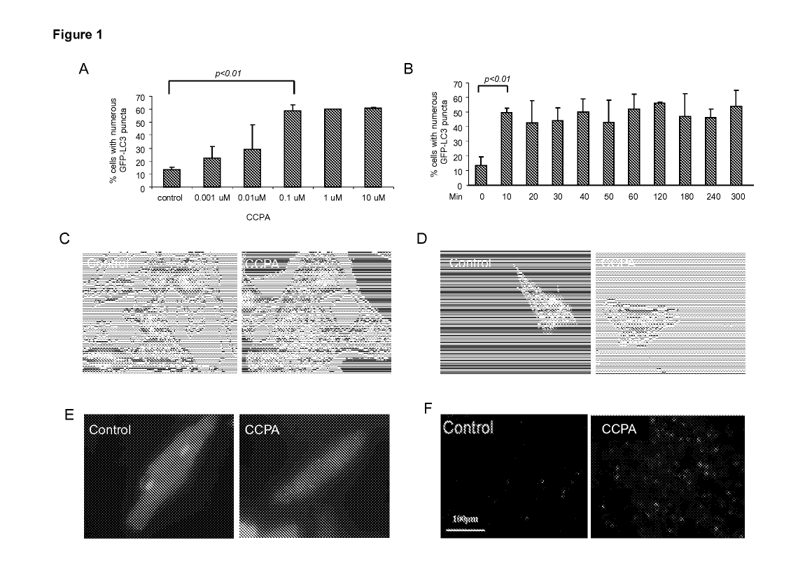

Adenosine receptor-selective effects on autophagy. We assessed the role of the adenosine A1 receptor using the selective agonist CCPA. As shown in FIG. 1, CCPA induced autophagy in a dose-dependent fashion. Autophagy was upregulated within 10 minutes after the addition of CCPA, and was sustained for several hours, consistent with the kinetics of the preconditioned state. We observed an increase in the number of autophagosomes in response to CCPA in HL-1 cells (1C), neonatal rat cardiomyocytes (1D), adult cardiomyocytes (1E), and in vivo in the hearts of mCherry-LC3 transgenic mice (1F).

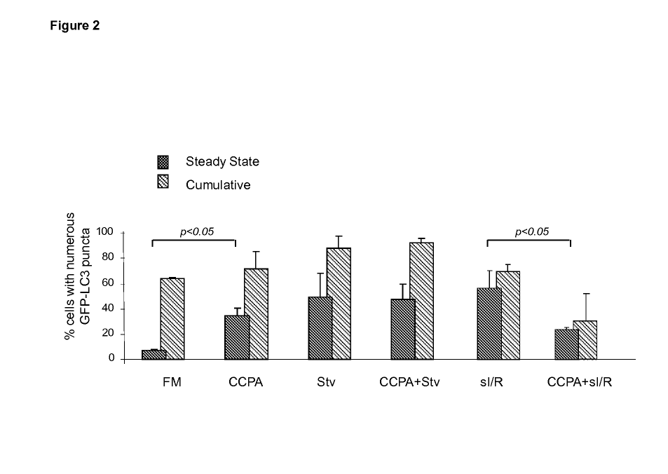

Effect of CCPA on autophagic flux under conditions of starvation or sI/R. An increase in the number of autophagosomes can be due to increased formation of autophageosomes or a decrease in their clearnace through lysosomal degradation. To measure flux, we inhibited autophagosomal degradation with Bafilomycin A1: an increase in the abundance of autophagosomes compared with steady state conditions (no Bafilomycin) reflects increased production. As shown in FIG. 2, CCPA increased the percentage of cells with numerous autophagosomes under both steady-state and cumulative conditions, indicating that CCPA increases autophagy rather than interfering with degradation. CCPA has no effect on the extent of autophagy induced by starvation. Simulated ischemia and reperfusion (sI/R) results in an increase in the percentage of cell s with numerous autophagosomes seen under steady state conditions, but this is due to impaired clearance rather than increased formation, as there is no significant increase in the number in the presence of Bafilomycin. Fewer autophagosomes were observed after sI/R in CCPA-treated cells. Since CCPA did not reduce autophagic flux indicated by starvation, it likely does not interfere with formation of autophagosomes in response to sI/R. If autophagy is upregulated during sI/R in an attempt to respond to the stress of nutrient deprivation and oxidants, then the diminished autophagy seen in CCPA-treated cells after sI/R may indicated that the cells experienced less stress, and therefore less autophagy is required during reperfusion (reparative autophagy).

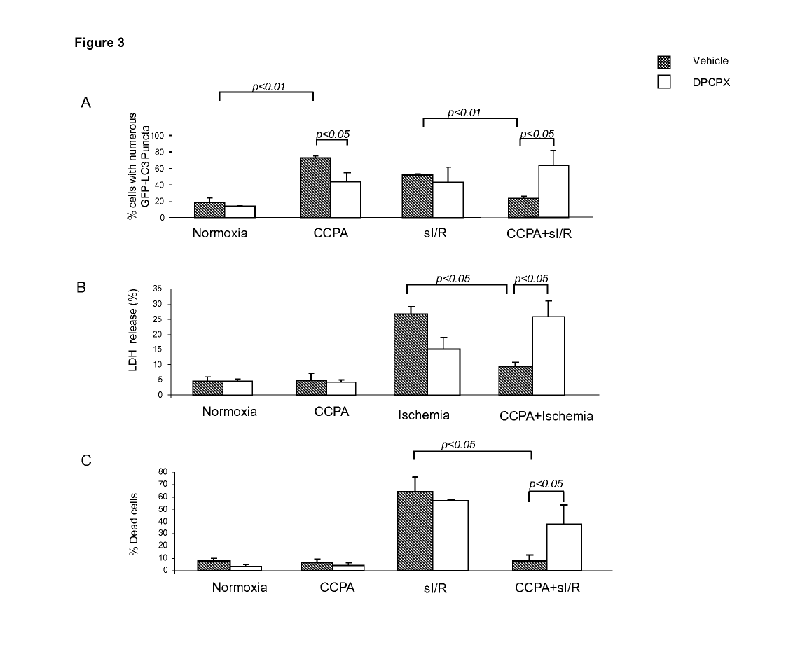

Receptor-selective effect of CCPA on autophagy and cytoprotection. To confirm that the effects of CCPA were mediated through the adenosine A1 receptor, HL-1 cells were treated with CCPA in the presence or absence of the A1 receptor antagonist DPCPX under conditions of normoxia or sI/R. As shown in FIG. 3, the upregulation of autophagy by CCPA under normoxic conditions was partially blocked by DPCPX. As expected, CCPA protected cells against sI/R as indicated by diminished LDH release and uptake of propidium iodide. Cytoprotection was abolished by DPCPX and the amount of autophagy during reperfusion, which we interpret to mean that there was more damage—hence more repair autophagy needed during reperfusion. These results suggest that the effects of CCPA on autophagy and cytoprotection are mediated through the adenosine A1 receptor.

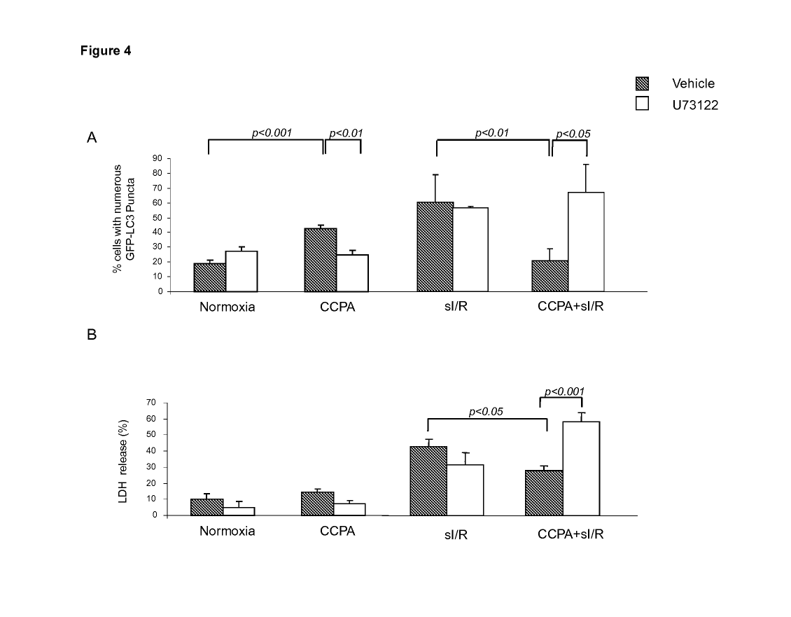

CCPA signals autophagy through PLC and a rise in intracellular calcium. The adenosine A1 receptor is a G-protein-coupled receptor that activates phospholipase C (PLC)²⁴. To determine if PLC signaling was upstream of autophagy induction by CCPA, we used the PLC inhibitor U73122 and assessed effects on autophagy and cytoprotection. As shown in FIG. 4, PLC is required for CCPA stimulation of autophagy before ischemia; blockade of the CCPA signal through PLC results in an increase in autophagy after sI/R (repair autophagy) as well as an increase in LDH release at end of stimulated ischemia.

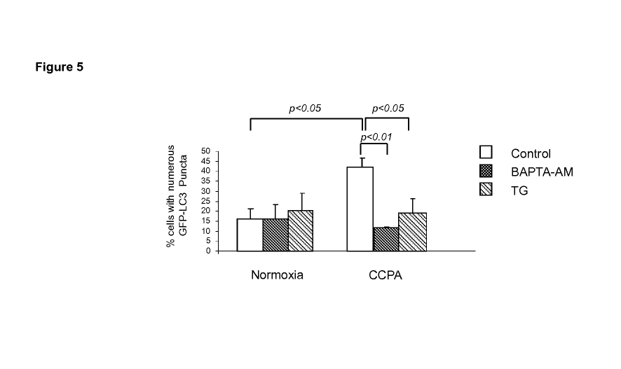

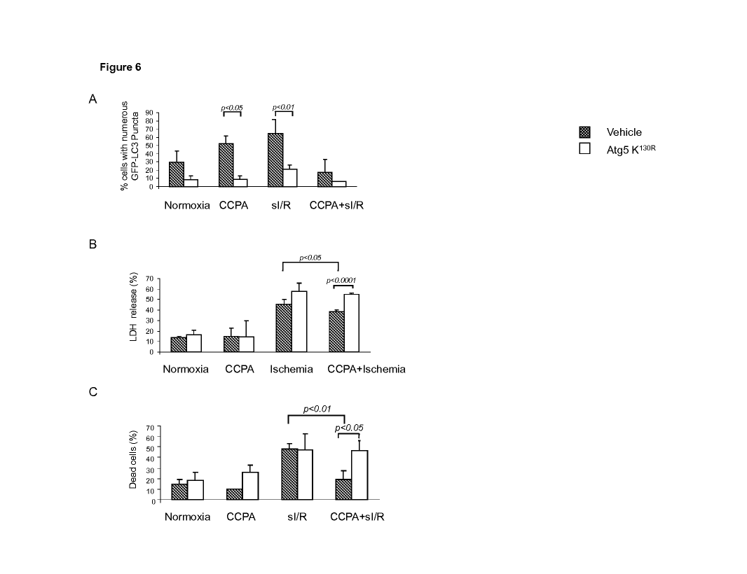

Autophagy (induced by starvation or rapamycin) is dependent upon on the release of calcium from the sarcoendoplasmic reticulum (S/ER)²⁵ as is adenosine preconditioning²⁶. As shown in FIG. 5, we confirmed that chelation of cytoplasmic calcium with BAPTA-AM, or depletion of S/ER calcium stores by thapsigargin pretreatment, suprressed the induction of autophagy by CCPA, suggesting a convergence of the two processes. This is consistent with our previous findings that starvation-induced autophagie flux is also suppressed by BAPTA or thapsigargin²⁵.

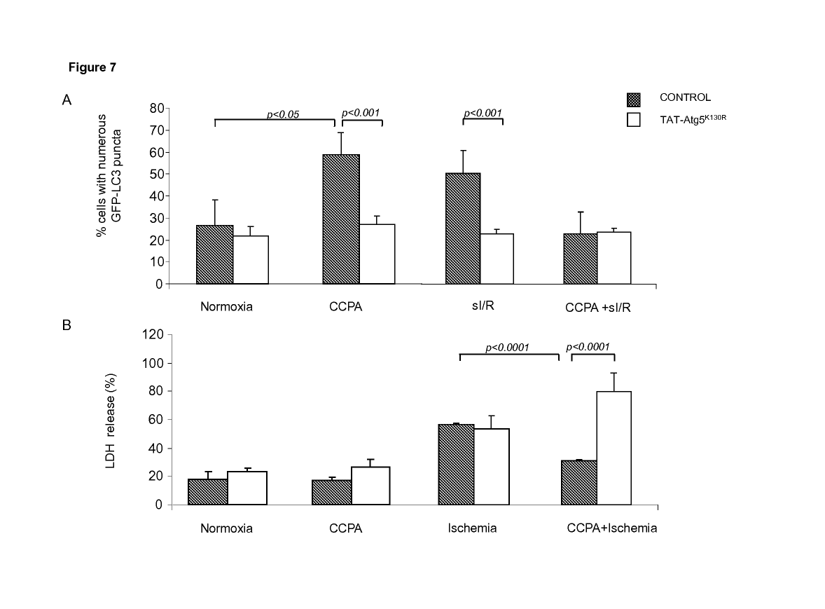

Cytoprotection by CCPA is dependent upon autophagy. The foregoing results were consistent with the notion that the CCPA-mediated induction of autophagy before sI/R was cytoprotective and resulted in a diminished need for autophagy after sI/R. We have previously shown that nitochondrial damage induces autophagy as part of a repair response^{11,27}. To determine whether autophagy is required for protection mediated by CCPA, we transfected HL-1 cells with a dominant negative inhibitor of autophagy (Atg5^{K130R}) or with empty vector. We confirmed that Atg5^{K130R} effectively suppressed autophagy (FIG. 6). Importantly, the dominant negative inhibitor of autophagy eliminated the protective effects of CCPA after sI/R. Direct suppression of autophagy was not cytoprotective, arguing against a deleterious role for autophagy, as has been suggested by some investigators. To further validate these findings, we performed this study in adult cardiomyocytes, using cell-permeable recombinant Tat-Atg5^{K130R} to inhibit autophagy. As shown in FIG. 7, CCPA induced autophagy in adult cardiomyocytes and conferred cytoprotection. Administration of Tat-Atg5^{K130R} suppressed autophagy and eliminated the protection by CCPA. It is important to note that inhibiting autophagy in the absence of CCPA did not increase LDH release under normoxic conditions nor did it exacerbate injury from sI/R, indicating that the recombinant protein is not directly cytotoxic. It also indicates that inhibiting autophagy is not protective in this cell culture model. These results provide clear and compelling evidence in support of the notion that CCPA mediates it cytoprotective effect through the induction of autophagy.

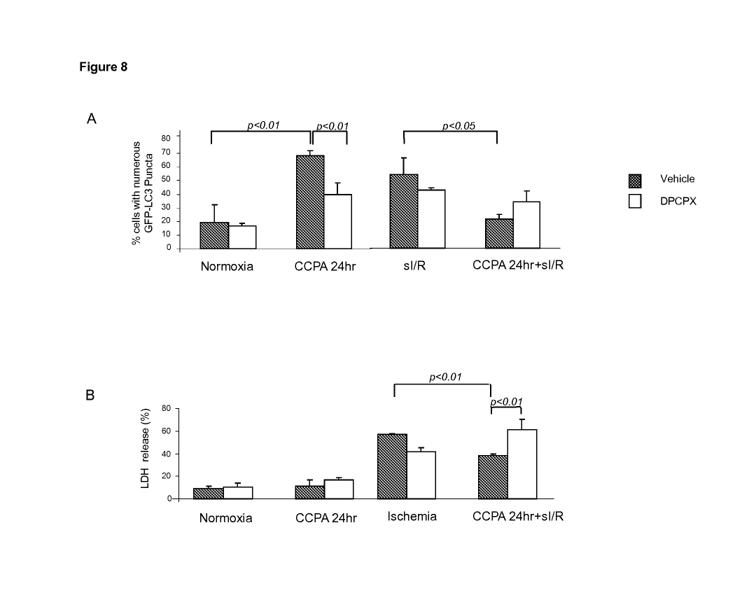

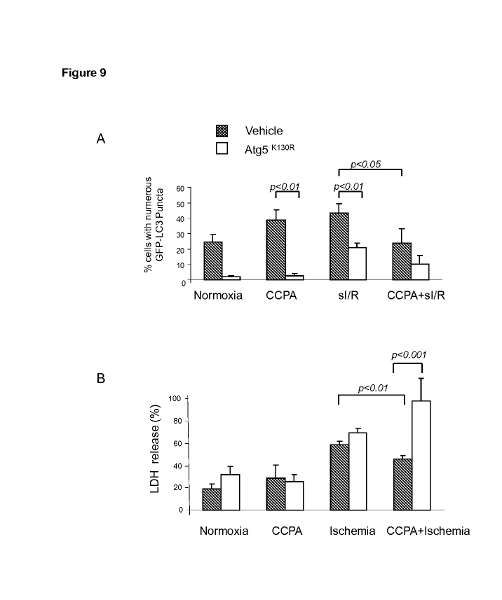

Effect of CCPA on delayed preconditioning. There are two windows of preconditioning: one is induced within minutes and lasts several hours, and the second window of protection is observed 16-24 hr after the preconditioning stimulus (delayed or late phase). We treated HL-1 cells with CCPA for 10 min in the presence or absence of DPCPX, then 24 hr later assessed autophagy and cytoprotection. As shown in FIG. 8, we found that autophagy is upregulated 24 hr after treatment with CCPA; as previously noted for immediate preconditioning, the amount of repair autophagy seen at reperfusion is less in CCPA-treated cells, reflecting less damage. The A1 antagonist blocked the effects of CCPA on autophagy and also abolished the cytoprotection by CCPA in the second window of protection. To determine if autophagy was required for the second window of protection, we transfected HL-1 cells with Atg5^{K130R}, the dominant negative inhibitor of autophagy. Atg5^{K130R} suppressed autophagy in the second window of protection and abolished the cytoprotective effect of CCPA (FIG. 9). Taken together, these results indicate that CCPA mediates delayed preconditioning by a mechanism that requires autophagy.

### Discussion

The role of autophagy in the heart is controversial, with some findings suggesting it may be deleterious while other studies suggest a clear protective role. Ischemic and pharmacologic preconditioning are recognized as the most potent and reproducible cardioprotective interventions yet identified, but the precise intracellular mechanism remains elusive. Based on our previous observation that autophagy is upregulated during reperfusion and serves a cytoprotective role in HL-1 cells, we hypothesized that autophagy might represent a component of the mechanism of preconditioning. To test this, we relied on the HL-1 myocyte cell line, which we have evaluated in a number of studies and have found to behave nearly identically to neonatal rat cardiomyocytes with respect to the autophagic response to sI/R¹, hydrogen peroxide²⁸, lipopolysaccharide²⁸, and pharmacologic preconditioning agents including CCPA. We also showed for the first time that CCPA upregulated autophagy in adult rat cardiomyocytes and in vivo in αMHC-mCherry-LC3 transgenic mice.

In HL-1 cells, we found that CCPA upregulated autophagy within 10 minutes, and conferred cytoprotection against sI/R in the same time frame. Interestingly, the amount of autophagy observed during the reperfusion phase was less than in untreated cells subjected to sI/R. This seemingly paradioxical effect can be explained if one considers autophagy part of a repair response. In preconditioned cells, less damage occurs during ischemia, so less repair autophagy is required during the reperfusion phase. If CCPA directly suppressed autophagy, one would expect it to suppress starvation-induced autophagy, but in that setting, it has no effect. Previous studies examining the abundance of autophagosomes in tissue have failed to take into account the turnover of these transient organelles. However, an increase in autophagosomes could be due to increased production or diminished clearnace through the lysomal pathway. We used comparisons of autophagy in the absence (steady-state) and presence (cumulative) of bafilomycin A1, which prevents autophagosome-lysosome fusion, in order to assess flux. Notably, the increase in autophagy observed after sI/R is largely due to impaired clearance (no increase in the presence of Baf). CCPA increases flux before sI/R, but appears to diminish autophagosome formation after sI/R without improving clearance (no increase after Baf).

Adenosine receptor signaling has been studied extensively and a variety of selective agonists and antagonists have been developed. CCPA is generally regarded as an A1-selective agonist, and DCPCX and A1-selective antagonist. We confirmed that the effects of CCPA on autophagy and on cytoprotection were mediated through the A1 receptor. We also confirmed that the downstream activation of phospholipase C and release of S/ER Ca⁺² were required for the effects on autophagy and cytoprotection.

Previous efforts to understand the role of autophagy in the heart have used Atg5(−/−) mice or Beclin1 (+/−) mice. The Atg5(−/−) mice develop a dilated cardiomyopathy, suggesting that autophagy plays an important role in normal cardiac homeostatis. The Beclin 1 (+/−) mice have diminished autophagy, and a previous study by Sadoshima's group indicated that these mice had smaller infarcts than their wild type littermates²⁹. However, this result must be interpreted with caution. It is unknown whether other compensatory pathways are upregulated in these animals; for instance, Atg5(−/−) mice show upregulation of ERK phosphorylation that is the basis for cytoprotection³⁰. Furthermore, Beclin 1 contains a BH3 domain which is postulated to function as a proapoptotic molecule. Reduction in the abundance of a proapoptotic protein may confer protective benefit independent of effects of autophagy. However, autophagy may not be universally protective, and its connection to innate immunity implies that perturbations to autophagy (up or down) may have pleiotropic effects^{28, 31, 32}.

As noted earlier, pharmacologic inhibitors of autophagy (3-MA and wortmannin) are nonspecific and may lead to confounding results. To overcome these concerns, we used a dominant negative inhibitor of autophagy, Atg5^{K130R}. We found that transient transfection of Atg5^{K130R} potently reduced autophagy and blocked the cytoprotective effect of CCPA in HL-1 cells subjected to sI/R. In the present study, cell death after sI/R was not increased by Atg5^{K130R}, in contrast to our previous findings¹. However, the studies differ with respect to readout (LDH release of both transfected and non-transfected cells versus Bax translocation scored only in transfected cells), and sensitivity (detection of small differences in cell viability is better in the Bax assay). However, the present results suggest that operational autophagy may not be essential to the basal/innate resilience to cardiomyocyte ischemia, but is important to the enhanced cytoprotection mediated by CCPA.

CCPA also elicits delayed preconditioning; we found upregulation of autophagy at 24 hr after a 10 min exposure to CCPA followed by washout. The effects on autophagy and cytoprotection against sI/R were receptor dependent, as they were blocked by DPCPX. The protective effects of CCPA in delayed preconditioning also depended on autophagy, as suppression of autophagy by Atg5^{K130R} abolished the cytoprotection.

In practicing this invention, other preconditioning agents may be used elicit autophagy, and for cardioprotection, e.g., as a pretreatment or during reperfusion, or postconditioning. We have shown that CCPA induces autophagy in the hearts of mCherry-LC3 mice. The present study demonstrates, for the first time, that autophagy serves as a key mediator of protection by the adenosine A1 receptor agonist CCPA. Thus, the autophagy-targeted compositions of this invention represent new therapeutic modalities.

### Figure Legends

FIG. 1. Adenosine receptor-selective effects on autophagy. (A) GFP-LC3 transfected HL-1 cells were treated for 120 min in full medium (FM) with various concentrations (0.001-10 μM) of CCPA. (B) GFP-LC3-transfected HL-1 cells were treated with 100 nM CCPA for the indicated time, then fixed with paraformaldehyde and scored by fluorescence microscopy. (C) Representative images of HL-1 cells expressing GFP-LC3, which is diffuse in quiescent cells and punctate in CCPA-treated cells (PC). (D) Representative images of neonatal cardiomyocytes under control conditions or 10 min after administration of 100 nM CCPA. (E) Representative images of adult cardiomyocytes under control conditions or 10 min after administration of 100 nM CCPA. (F) Transgenic mice expressing mCherry-LC3 under the αMHC promoter received an i.p. injection of saline or 1 mg/kg CCPA, then were sacrificed 30 min later and heart tissue was processed for fluorescence microscopy. The increase in fluorescent red puncta reflects upregulation of autophagy.

FIG. 2. Effect of CCPA on autophagic flux under conditions of starvation or sI/R. HL-1 cells were infected with adv-GFP-LC3, treated with or without 100 nM CCPA in full medium (FM) for 10 min, then subjected either to starvation (amino acid deprivation in MKH) (Stv) for 3 hr, or simulated I/R (2 hr sI, 3 hr R). Steady-state and cumulative conditions were assessed by incubating cells with or without the lysosomal inhibitor Bafilomycin during the starvation or reperfusion phase. The extent of autophagy was assessed by the intracellular distribution of GFP-LC3 by fluorescence microscopy. The experiments were done at least three times and results shown are mean ±SEM.

FIG. 3. Receptor-selective effect of CCPA on autophagy and cytoprotection. Adv-GFP-LC3 infected HL-1 cells were treated in full medium with the selective A1 receptor antagonist DPCPX for 30 min, followed by 100 nM CCPA for 10 min, and then cells were subjected to sI/R (2 hr sI, 3 hr R). The extent of autophagy was assessed by the intracellular distribution of GFP-LC3 by fluorescence microscopy (A), and cell death was measured by LDH release at the end of simulated ischemia (B) or by propidium iodide uptake at the end of reperfusion (C).

FIG. 4. CCPA signals autophagy through PLC. HL-1 cells infected with Adv-GFP-LC3 were treated with the PLC inhibitor U73122 (2 μM) for 15 min followed by CCPA for 10 min, then incubated in normoxic conditions or subjected to sI/R (2 hr sI, 3 hr R). Autophagy was scored by fluorescence microscopy (A). The amount of LDH released to the medium was determined immediately after ischemia and compared to the total activity of control homogenate (100%) (B).

FIG. 5. CCPA signals autophagy through a rise in intracellular calcium. HL-1 cells were treated with 1 μM thapsigargin (TG) or 25 μM BAPTA-AM for 15 min followed by CCPA for 10 min. The cells were washed in PBS and fixed and the intracellular distribution of GFP-LC3 was assessed by fluorescence microscopy.

FIG. 6. Cytoprotection by CCPA id dependent upon autophagy. HL-1 cells were co-transfected with GFP-LC3 and the dominant negative autophagy protein Atg5^{K130R}. After 24 hr cells were treated for 10 min with CCPA followed by sI/R (2 hr sI, 3 hr R). The extent of autophagy was assessed by the intracellular distribution of GFP-LC3 by fluorescence microscopy (A). Cytoprotection was assessed by measuring LDH released into the media at the end of ischemia (B) or by propidium iodide uptake (C).

FIG. 7. Cytoprotection by CCPA requires autophagy in adult cardiomyocytes. Adult rat cardiomyocytes were infected with GFP-LC3 adenovirus for 2 hours and washed with the plating medium. After 20 hr, cells were incubated with or without Tat-Atg5^{K130R} for 30 min followed by CCPA or vehicle for 10 min. Cells were subjected to normoxia or simulated ischemia followed by 2 hr reperfusion, and autophagy was scored as the percentage of cells with numerous puncta (A). For determination of cell death, LDH release into the culture supernatant was measured at the end of simulated ischemia (B).

FIG. 8. Receptor-selective stimulation of autophagy in delayed preconditioning. GFP-LC3 infected HL-1 cells were treated with the selective A1 receptor antagonist DPCPX for 30 min prior to CCPA exposure for 10 min followed by washout. After 24 hr, the cells were exposed to sI/R (2 hr sI, 3 hr R). The cells were fixed, and the extent of autophagy was assessed by the intracellular distribution of GFP-LC3 by fluorescence microscopy in normoxia and after sI/R (A). Cell death was measured by LDH release at the end of ischemia (B).

FIG. 9. Role of autophagy in delayed preconditioning. HL-1 cells were co-transfected with GFP-LC3 and dominant negative Atg5^{K130R}. Cells were treated with CCPA for 10 min, followed by washout. 20 hr later, cells were subjected to sI/R (2 hr sI, 3 hr R). The extent of autophagy was assessed by the intracellular distribution of GFP-LC3 by fluorescence microscopy (A) and cell death was measured by LDH release into the medium at the end of ischemia (B).

### REFERENCES

### Example 1

1. Hamacher-Brady A, Brady N R, Gottlieb R A. Enhancing macroautophagy protects against ischemia/reperfusion injury in cardiac myocytes. J Biol Chem. 2006;281 (40):29776-29787.
2. Cohen M V, Downey J M. Adenosine: trigger and mediator of cardioprotection. Basic Res Cardiol. 2008; 103(3):203-215.
3. Downey J M, Krieg T, Cohen M V. Mapping preconditioning's signaling pathways: an engineering approach. Ann N Y Acad Sci. 2008; 1123:187-196.
4. Yao Z, Gross G J. A comparison of adenosine-induced cardioprotection and ischemic preconditioning in dogs. Efficacy, time course, and role of KATP channels, Circulation. 1994;89(3):1229-1236.
5. R M, Eells J T, Hsu A K, Henry M M, Gross G J. Ischemic preconditioning in rats: role of mitochondrial K (ATP) channel in preservation of mitochondrial function. Am J Physiol Heart Circ Physiol. 2000;278(1): H305-312.
6. Costa A D, Garlid K D. Intramitochondrial signaling: interactions among mitoKATP, PKCepsilon, ROS, and MPT. Am J Physiol Heart Circ Physiol. 2008;295(2):H874-882.
7. Garcia-Dorado D, Rodriguez-Sinovas A, Ruiz-Meana M, Inserte J, Agullo L, Cabestrero A. The end-effectors of preconditioning protection against myocardial cell death secondary to ischemia-reperfusion. Cardiovasc Res. 2006;70(2):274-285.
8. Caro L H P, Plomp P J A M, Wolvetang E J, Kerkhof C, Meijer A J. 3-Methyladenine, an inhibitor of autophagy, has multiple effects of metabolism, European Journal of Biochemistry. 1988;175(2):325-329.
9. Sckulie A, Hudson C C, Homme J L, Yin P, Otterness D M, Karnitz L M, Abraham R T. A Direct Linkage between the Phosphoinositide 3-Kinase-AKT Signaling Pathway and the Mammalian Target of Rapamycin in Mitogen-stimulated and Transformed Cells. Cancer Research. 2000;60(13):3504-3513.
10. Mizushima N, Noda T, Yoshimori T, Tanaka Y, Ishii T, George M D, Klionsky D J, Ohsumi M, Ohsumi Y. A protein conjugation system essential for autophagy Nature. 1998;395(6700):395-398.
11. Hamacher-Brady A, Brady N R, Logue S E, Sayen M R, Jinno M, Kirshenbaum L A, Gottlieb R A, Gustafsson A B. Response to myocrodial ischemia/reperfusion injury involves Bnip3 and autophagy. Cell Death Differ. 2006:14:146-157.
12. Pyo J-O, Jang M-H, Kwon Y-K, Lee H-J, Jun J-IL, Woo H-N, Cho D-H, Choi B, Lee H, Kim J-H, Mizushima N, Oschumi Y, Jung Y-K. Essential Roles of Atg5 and FADD in Autophagic Cell Death: DISSECTION OF AUTOPHAGIC CELL DEATH INTO VACUOLE FORMATION AND CELL DEATH. J. Biol. Chem. 2005;280(21):20722-20729.
13. Decker R S, Wildenthal K. Lysosomal alterations in hypoxic and reoxygenated hearts. I. Ultrastructural and cytochemical changes. Am J Pathol. 1980;98(2):425-444.
14. Kabeya Y, Mizushima N, Ueno T, Yamamoto A, Kirisako T, Noda T, Kominami E. Ohsumi Y, Yoshimori T, LC3, a mammalian homologue of yeast Apg8p, is localized in autophagosome membranes after processing. Embo J. 2000;19(21):5720-5728.
15. Yan L, Vatner D E, Kim S J, Ge H, Masurekar M, Massover W H, Yang G, Matsui Y, Sadoshima J, Vatner S F. Autophagy in chronically ischemic myocardium. Proc Natl Acad Sci USA. 2005.
16. Claycomb W C, Lanson N A, Jr., Stallworth B S, Egeland D B, Delcarpio J B, Bahinski A, Izzo N J, Jr. HL-1 cells; a cardiac muscle cell line that contracts and retains phenotypic characteristics of the adult cardiomyocyte. Proc Natl Acad Sci USA. 1998;95(6):2979-2984.
17. Becker-Hapak M, McAllister S S, Dowdy S F, TAT-Mediated Protein Transduction into Mammalian Cells, Methods. 2001;24(3):247-256.
18. Gustafsson A B, Sayen M R, Williams S D, Crow M T, Gottlieb R A. TAT protein transduction into isolated perfused hearts; TAT-apoptosis repressor with caspase recruitment domain is cardioprotective. Circulation 2002;106(6):735-739.
19. Yamamoto A, Tagawa Y, Yoshimori T, Moriyama Y, Masaki R, Tashiro Y. Bafilomycin A1 prevents maturation of autophagic vacuoles by inhibiting fusion between autophagosomes and lysosomes in rat hepatoma cell line, B-4-II-E cells. Cell Struct Funct. 1998;23(1):33-42.
20. El-Ani D, Jacobson K A, Shainberg A. Characterization of adenosine receptors in intact cultured heart cells. Biochemical Pharmacology. 1994;48(4):727-735.
21. Safran N, Shneyvays V, Balas N, Jacobson K A, Nawrath H, Shainberg A. Cardioprotective effects of adenosine A1 and A3 receptor activation during hypoxia in isolated rat cardiac myocytes. Mol. Cell Biochem. 2001;217(1-2):143-152.
22. Nieminen A-L, Gores G J, Bond J M, Imberti R, Herman B, Lemasters J J. A novel cytotoxicity screening assay using a multiwell fluorescence scanner. Toxicology and Applied Pharmacology, 1992;115(2):147-155.
23. Iwai-Kanai e, Yuan H, Huang c, Sayen M R, Perry-Garza C N, Kim L, Gottlieb R A. A method to measure cardiac autophagic flux in vivo, Autophagy. 2008;4(3):322-329.
24. Ethier M F, Madison J M. Adenosine A1 receptors mediate mobilization of calcium in human bronchial smooth muscle cells. Am J Repir Cell Mol Biol. 2006;35(4):496-502.
25. Brady N R, Hamacher-Brady A, Yuan H, Gottlieb R A. The autophagic response to nutrient deprivation in the HL-1 cardiac myocyte is modulated by Bel-2 and sarco/endoplasmic reticulum calcium stores. FEBS Journal. 2007;274(12):3184-3197.
26. Mubagwa K. Does adenosine protect the heart by acting on the sarcoplasmic reticulum? Cardiovasc Res. 2002;53(2):286-289.
27. Hamacher-Bradt A, Brady N R, Gottlieb R A, Gustafsson A B. Autophagy as a protective response to Bnip3-mediated apoptotic signaling in the heart. Autophagy. 2006;2(4):307-309.
28. Yuan H. Perry C N, Huang C, Iwai-Kanai E, Carreira R S, Glembotski C C, Gottlieb R A, LPS-Induced Autophagy Is Mediated by Oxidative Signaling in Cardiomyocytes and is Associated with Cytoprotection. Am J Physiol Heart Circ Physiol. 2008.
29. Takagi H, Matsui Y, Hirotani S, Sakoda H, Asano T, Sadoshima J. AMPK Mediates Autophagy During Myocardial Ischemia In Vivo, Autophagy. 2007;3(4):405-407.
30. Sivaprasad U, Basu A. Inhibition Of ERK Attenuates Autophagy And Potentiates Tumor Necrosis Factor-alpha-Induced Celll Death In MCF-7 Cells. J Cell Mol Med. 2008.
31. Valeur H S, Valen G. Innate immunity and myocardial adaptation to ischemia. Basic Res Cardiol. 2009;104(1):22-32.
32. Saitoh T, Fujita N, Jang M H, Uematsu S, Yang B G, Satoh T, Omori H, Noda T, Yamamoto N, Komatsu M, Tanaka K, Kawai T, Tsujimura T, Takeuchi O, Yoshimori T, Akira S. Loss of the autophagy protein Atg16L1 enhances endotoxin-induced IL-1beta production. Nature. 2008;456(7219):264-268.
33. Hausenloy D J, Wynne A M, Yellon D M. Ischemic preconditioning targets the reperfusion phase. Basic Res Cardiol. 2007;102(5):445-452.
34. Mykytenko J, Reeves J G, Kin H, Wang N P, Zatta A J, Jiang R, Guyton R A, Vinten-Johansen J, Zhao Z Q, Persistent beneficial effect of postconditioning against infarct size; role of mitochondrial K(ATP) channels during reperfusion. Basic Res Cardiol. 2008;103(5):472-484.
35. Sivaraman V, Mudalagiri N R, Di Salvo C, Kolvekar S, Hayward M, Yap J, Keogh B, Hausenloy D J. Yellon D M. Postconditioning protects human atrial muscle through the activation of the RISK pathway. Basic Res Cardiol. 2007;102(5):453-459.

### Example 2

### Autophagy and Protein Kinase C Are Required for Cardioprotection By Sulfaphenazole

The following example describes making and using exemplary polypeptides of this invention, and demonstrates their efficacy.

Previously we showed that sulfaphenazole (SUL), an antimicrobial agent that is a potent inhibitor of cytochrome P4502C9, is protective against ischemia/reperfusion (I/R) injury. The mechanism, however, underlying this cardioprotection, is largely unknown. With evidence that activation of autophagy is protective against simulated I/R in HL-1 cells, and evidence that autophagy is upregulated in preconditioned hearts, we hypothesized that SUL-mediated cardioprotection might resemble ischemic preconditioning with respect to activation of protein kinase C and autophagy. We used the Langendorff model of global ischemia to assess the role of autophagy and protein kinase C in myocardial protection by SUL during I/R.

We show that SUL enhanced recovery of function, reduced creatine kinase release, decreased infarct size, and induced autophagy. SUL also triggered PKC translocation, whereas inhibition of PKC with chelerythrine blocked the activation of autophagy in adult rat cardiomyocytes. In the Langendorff model, ehelerythrine suppressed autophagy and abolished the protection mediated by SUL. SUL increased autophagy in adult rat cardiomyocytes infected with GFP-LC3 adenovirus, in isolated perfused rat hearts, and in mCherry-LC3 transgenic mice.

To establish the role of autophagy in cardioprotection, we used the exemplary cell-permeable dominant negative inhibitor of autophagy, Tat-Atg5^{K130R} of the invention. Autophagy and cardioprotection were abolished in rat hearts perfused with recombinant Tat-Atg5^{K130R}. Taken together, these studies indicate that cardioprotection mediated by SUL involves a PKC-dependent induction of autophagy. The findings suggest that autophagy may be a fundamental process that enhances the heart's tolerance to ischemia.

We recently reported that autophagy appears to be a necessary process involved in the cardioprotection conferred by 2-chloro-N⁶-cyclopentyladenosine (CCPA), an adenosine receptor A₁ agonist that has been shown to mimic ischemic preconditioning (45). Because of the possibility that SUL might share a common mechanism with CCPA and with ischemic preconditioning, we elected to investigate the role of autophagy in the myocardial protection afforded by SUL. Many studies of cardioprotection have demonstrated a role for protein kinase C. While there is controversy over the roles of various isozymes, most studies agree that chelerythrine blocks preconditioning mediated by a variety of inducing stimuli (3, 10, 11). I/R injury is associated with the formation of protein aggregates and damaged mitochondria which can only be removed by autophagy. Autophagy may also benefit the cell by generating metabolic substrates (amino acids, free fatty acids, and glycogen) from intracellular stores through breakdown of proteins, organelles, and glycogen granules. For these reasons we considered it likely that protection mediated by SUL would involve autophagy.

### Materials and Methods

Langendorff perfusion. The isolated perfused rat heart model was utilized as previously described (8, 16). In brief after anesthesia and heparinization (pentobarbital sodium 60 mg/kg i.p. and heparin 500 U i.p.), rat hearts were excised into ice cold Krebs-Henseleit solution (mM 118.5 NaCl, 4.7 KCl, 1.18 KH₂PO₄, 1.18 MgSO₄, 25 NaHCO₃, 11.1 glucose, 2.5 CaCl₂) and perfused with oxygenated buffer within 30 s. Hearts were perfused at constant pressure (60 mm Hg) for 5 min before administration of any drugs. Where indicated, sulfaphenazole dissolved in dimethyl sulfoxide (SUL, 10 μM) was administered throughout the perfusion. For hemodynamic analysis, a balloon made by plastic wrap was inserted into the ventricle through the left atrium. Hemodynamic parameters were recorded with the EMKA system. All procedures were approved by the Animal Care and Use Committee at The Scripps Research Institute and at San Diego State University, and conform to the Guide for the Care and Use of Laboratory Animals (National Institutes of Health publication no. 85-23, revised 1996).

Tat-Atg5^{K130R} (approximately 200 nM), or Tat-beta-galactosidase (Tat-β-gal, approximately 200 nM) was infused for 15 min before ischemia. Inhibition of autophagy was accomplished using the exemplary cell-permeable agent, TAT-Atg5^{K130R}, to selectively inhibit autophagy. This was necessary because the two widely-used inhibitors of autophagy, 3-methyladenine and wortmannin, have broad non-specific effects that can confound the interpretation of the results. 3-methyladenine alters intermediary metabolism and could have beneficial effects unrelated to its effects on autophagy (6), and wortmannin will inhibit not only the PI3-kinase involved in regulating autophagy, but also the PI3-kinase that is responsible for activating Akt (1, 33).

Where indicated, chelerythrine was added for 15 min before the onset of ischemia. Control hearts were perfused with a similar amount of DMSO (final concentration 0.01%). Global no-flow ischemia was maintained for 30 min, and reperfusion was accomplished by restoring flow. CK release was measured in the coronary effluent of the first 15 min of reperfusion using the CK EC 2.7.3.2™ UV test kit (Stanbio Lab). Infarct size determination by triphenyl tetrazolium chloride (TTC) staining was performed on hearts reperfused for 120 min (8). Other biochemical analyses of ischemic and reperfused heart tissue were performed on hearts flash-frozen in liquid nitrogen at the times indicated.

Induction of autophagy in vivo and ex vivo. mCherry-LC3 transgenic mice were given SUL (10 mg/kg) or vehicle by i.p. injection; after 30 min, hearts were removed and processed for cryosections images and/or cadaverine assay. To quantitate the autophagosomes, cryosections were washed with PBS for 5 min. Red dots (mCherry-LC3-labeled autophagosomes) were then counted under the microscope. Hearts were subjected to global no-flow ischemia for 30 min followed by 120 min reperfusion, then harvested and prepared for different assays as described below or TTC staining as described above.

Preparation of recombinant Tat-Atg5^{K130R}. Recombinant protein expression and purification was performed as described by Becker-Hapak et al. (4). Briefly, a 100 mL LB-amplicillin overnight culture of Tat-Atg5^{K130R} was grown at 37° C. and 225 rpm to an OD₆₀₀ of 0.9-1.2. The overnight culture was diluted into 1 L of fresh LB-ampicillin and incubated to an OD₆₀₀ of 0.6-0.9. 0.5 mM isopropylthiogalactoside (Roche) was added to the culture and incubated for an additional 3 h. The bacterial pellet was harvested by centrifugation at 6000 rpm for 15 min and resuspended in 20 mL 1× PBS. This was repeated twice with the final pellet dissolved in 15 mL buffer Z (8 M urea, 100 mM NaCl, and 20 mM Hepes, pH 8.0) and left overnight at 4° C. The lysate was sonicated on ice 3 times for 15 second pulses followed by centrifugation at 16000 rpm for 30 min. The supernatant was saved and equilibrated in 10 nM imidazole. Half was applied to a 25 mL column packed with 6 mL of Ni-NTA resin (Qiagen) equilibrated in buffer Z with 10 mM imidazole. The mixture was allowed to incubate at room temperature on a rocker for 1 hr. The suspension was collected by gravity flow and the flow through was re-applied onto the column twice. The column was washed with 50 mL of buffer Z containing 10 mM imidazole and proteins were eluted in buffer Z containing 250 mM imidazole followed by another elution with buffer Z containing 1 M imidazole. Both elution fractions were pooled together and concentrated to half the volume using an Amicon Ulta centrifugation device (Millipore). The proteins were then de-salted into 1× PBS plus 10% glycerol in 2.5 mL aliquots and eluted with 3.5 mL on a PD-10 column (GE Healthcare) and filtered through a 0.22 μm filter. 200 μL aliquots of purified fusion proteins were stored at −80° C. until use.

Isolation and treatment of adult rate cardiomyocytes. Isolation of adult rat cardiomyocytes was performed as previously described (21). Briefly, rat hearts were perfused with perfusion buffer (modified KHB buffer: 10 mM HEPES, 30 mM taurine, 2 mM carnitine and 2 mM creatine in 500 mL Joklik's MEM, pH 7.3) for 4 min at 3 ml/min and then digested with digestion buffer (1 mg/mL of collagenase II, 6.25 μM CaCl₂ in 50 mL perfusion buffer) for 18 min at 3 mL/min. The heart was then removed and minced in digestion buffer, to which Stop Buffer (perfusion buffer containing 12.5 μM CaCl₂ and 5% newborn calf serum) was added. Cells were allowed to sediment by gravity for 8-10 min in a 50 mL Falcon tube. The supernatant was removed and the pellet was resuspended in 30 mL of room temperature Stop Buffer. Calcium was then reintroduced to myocytes gradually to achieve a concentration of 1 mM while monitoring by microscopy. Rod shaped myocytes (100,000 per 2 mL) were plated in laminin-coated 35 mm dishes and allowed to recover for 6 hr. Cells were infected with GFP-LC3 adenovirus for 2 hr, washed, and cultured for 16 hr in full medium containing 10% fetal calf serum and 10% newborn calf serum before exposure to SUL and chelerythrine. Chelerythrine was added to medium at a final concentration of 5 μM 10 min before the addition of SUL. Cells were treated with 10 μM SUL for 30 min and autophagosomes (green dots) were quantified by fluorescence microscopy.

For assessment of subcellular distribution of PKC δ, rod shaped cardiomyocytes were plated in laminin-coated 35 mm MATTEK™ glass bottom dishes (14 mm glass microwell). Following 15 min treatment with SUL or vehicle (CON), cells were fixed with 4% paraformaldehyde for 15 min. Fixed cells were permeabilized with 0.3% Triton X-100/PBS for 10 min, blocked for 45 min in 3% BSA/0.3Triton X-100/PBS, and stained with mouse anti-α-actinin (Sigma) and rabbit anti-PKC δ (Sigma) and the respective secondary antibodies (mouse Alexa Fluor 488™ and rabbit Alexa Fluor 546™ (Invitrogen)). Imagining was performed at 60× magnification using a Nikon TE300™ fluorescence microscope.

Histological analysis and immunostaining. Hearts were embedded in OCT and 7 micron frozen sections were prepared. For immunostaining, tissue sections were immersed in acetone for 1-2 min at room temperature and then allowed to air dry. Samples were incubated in TBS buffer with 5% horse serum, 5% goat serum, and 0.3% Triton-X100 for 20 min and then incubated with primary antibody following manufacture instruction for 2 hr (1:200 of LC3 antibody from Novus Bio and 1:500 anti-HA from Santa Cruz). Stained sections were observed through a Nikon TE300™ fluorescence microscope (Nikon) equipped with a cooled CCD camera (Orca-ER, Hamamatsu).

Subcellular fractionation. Frozen heart samples were thawed on ice in homogenization buffer containing (in mmol/L): Tris-HCL 20, EDTA 2, EGTA 10, PMSF 1, leupeptin 0.1, E-64 0.01, and sucrose 250). The tissue was then minced and Plytron homogenized (Kinematica, Basel, Switzerland) on ice for 15s for three passes. The homogenates were centrifuged at 600 g for 5 min at 4° C, and the crude supernatants were further centrifuged at 10,000 g for 10 min 4° C. The supernatant, designated as crude cytosol, was divided and one fraction was further centrifuged at 100,000 g for 1 h at 4° C. The resulting supernatant was designated as cytosolic fraction. The pellet was resuspended in homogenization buffer with 1% TritonX-100, incubated on ice for 1 h, then centrifuged at 100,000 g for 1 h at 4° C. The resulting supernatant was designated as the particulate fraction. Samples were stored at −80° C. until use.

Western blot analysis. Proteins prepared from rat hearts were quantified by Bio-Rad protein assay. For immunodetection, 50 μg of crude cytosol prepared as above were resolved on SDS-PAGE 10% denaturing gels and transferred to PVDF nylon membranes. The membranes were blocked with 5% nonfat dry milk in TNT buffer (in mM: NaCl 100, Tris·HCl 10 (pH 7.4), and 0.1% Tween-20) for 1 h. The blots were then incubated with 200-fold diluted primary antibodies against LC3 (Novus Bio., Littleton, Colo.) at 4° C. overnight, or with 1,000-fold diluted primary antibodies against PKC δ (Sigma) and PKCε (BD) at room temperature for 2 h. Membranes were washed with TNT buffer at room T and incubated with appropriate peroxidase-conjugated secondary antibody (1:2000 dilution). Immunoreactive bands were visualized by chemiluminescence (ECL kit, Amersham) on X-ray film. Each immunoblotting experiment was repeated three to five times and the results were averaged. To quantify the protein, intensity of bands was assessed with Scion Image Software.

Measurement of autophagy by cadaverine uptake. Heart tissue from Langendorff-perfused rat hearts was minced in homogenization buffer (250 mM Sucrose, 1 mM Na₂EDTA, 10 mM HEPES, pH 7.0, plus fresh protease inhibitors), and homogenized by Polytron for 5 sec at half speed. Nuclei and heavy membranes were removed by centrifugation at 1000×g for 5 min at 4° C. The post-nuclear supernatant was moved to new 1.5 mL centrifuge tubes and incubated with Alexa Fluor 488 Cadaverine (Molecular Probes) at 25 μM final concentration for 10 min. The samples were spun at 20,000×g for 20 min at 4° C. and the pellet washed twice with resuspension buffer (140 mM KCl, 10 mM MgCl₂, 5 mM KH₂PO₄, 1 mM EGTA, 10 mM MOPS, pH 7.4 plus fresh protease inhibitors). The pellet was resuspended in 350 μL resuspension butter and the fluorescence intensity read on a 96-well plate reader at excitation/emission 495/519 nm in triplicate. The relative fluorescence units were standardized to the protein concentration of each sample which was determined by Bradford assay (Pierce).

We previously described the highly specific co-localization of monodansylcadaverine with mCherry-LC3 puncta (24), (46), and subsequently found that the labeling could be performed on frozen heart tissue or homogenates (38). We also found that AlexaFluor488™-cadaverine and BODIPY-TR™-cadaverine (Invitrogen) were preferable to monodansylcadaverine because of greater selectivity, lower background signal, improved fluorescence properties and slight improvement in the ability to preserve the signal after tissue fixation (38). These various approaches were consistent in their ability to reflect autophagy, and the advantage of the cadaverine incorporation method is that it can be used on frozen tissue samples and provides a quantitative result without the need for laborious point-counting of microscopy fields.

To further validate this method, we probed the pellet obtained after the 20,000×g spin for the presence of the autophagy marker protein, LC3. We detected LC3-II in the pellet (consistent with autophagosome membranes), and confirmed that the amount of LC3-II was proportional to the amount of cadaverine dye binding (data not shown).

Statistical analysis. Statistical analysis was performed between groups by ANOVA by using INSTAT 4.10 software (GraphPad™). A P value<0.05 was considered significant.

### Results

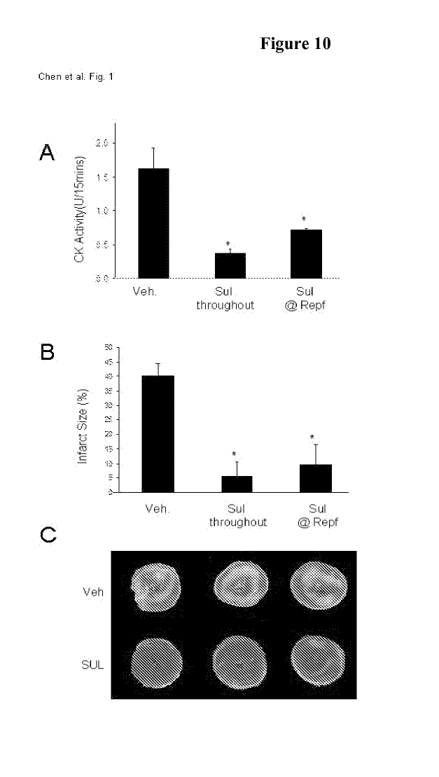

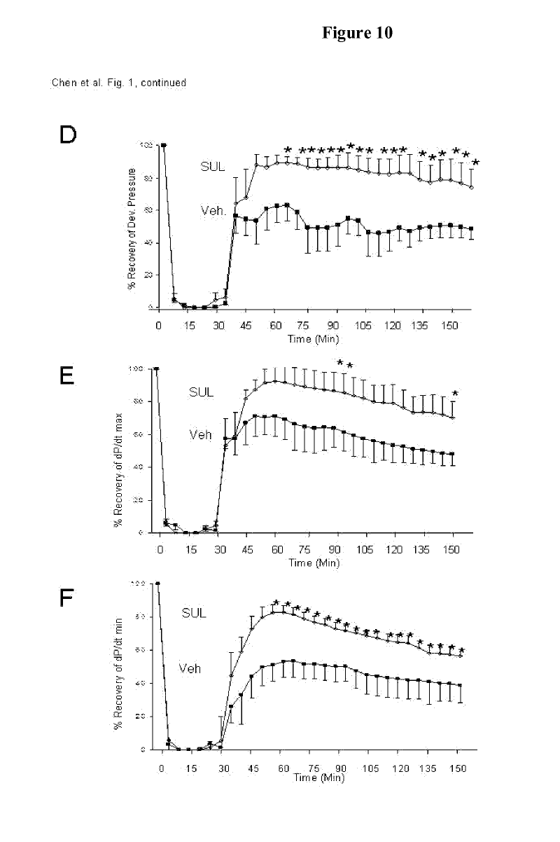

SUL protects isolated perfused rat hearts from I/R injury. Here, we confirmed our previous study that showed that sulfaphenazole attenuated CK release and reduced infarct size (15). We extended the findings to measure hemodynamics and infarct size using 10 μM SUL introduced into the perfusion buffer 10 min before ischemia and maintained throughout reperfusion, or added only at the onset of reperfusion. As shown in FIG. 10A-C, SUL administration attenuated CK release and reduced infarct size; the reduction of infarct size was sustained even when SUL was introduced at the onset of reperfusion. SUL had no effect on contractility before ischemia. SUL enhanced recovery of contractile function alter I/R to about 90% of pre-ischemic value, whereas vehicle control hearts recovered only to about 50% of pre-ischemic values (FIG. 10D-F),

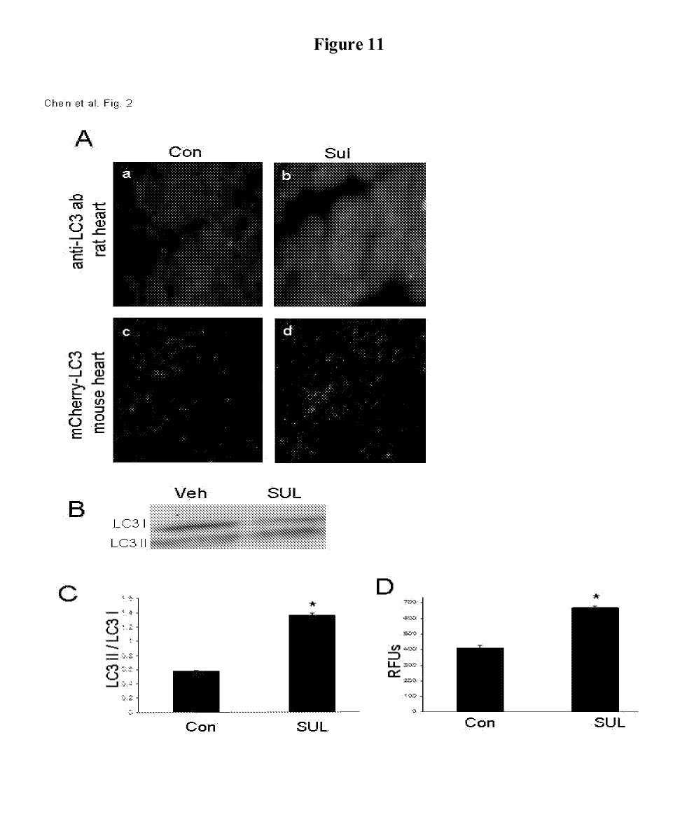

SUL induces autophagy. To determine whether SUL induced autophagy in the heart, isolated perfused rat hearts were exposed to SUL for 30 min and the distribution of autophagosomes (LC3 dots) was assessed by immunostaining (FIG. 11A, a and b). During the induction of autophagy, LC3 is proteolytically processed by Atg4 to expose a terminal glycine (LC3-1) and then is conjugated to phosphatidylethanolamine by Atg7, a specialized ubiquitin ligase. The lipidated LC3 is membrane-associated and has an altered mobility on SDS-PAGE (LC3-II). The conversion of LC3-I to LC3-II reflects autophagic flux. SUL administration resulted in a doubling of the ratio of LC3-II/I (FIGS. 11B and 11C).

To confirm that the autophagy was upregulated specifically in cardiomyocytes, we used mCherry-LC3 transgenic mice, in which the transgene is under the control of the α MHC promoter, thereby restricting expression of the red fluorescent LC3 fusion protein to cardiomyocytes. There was a significant increase in the number of autophagosomes in the hearts of SUL-treated mice (FIG. 11A, c and d) and quantified by cadaverine assay in FIG. 11D). These results demonstrate that SUL induced autophagy in adult rat cardiomyocytes, in the isolated perfused rat heart, and in the mouse heart in vivo.

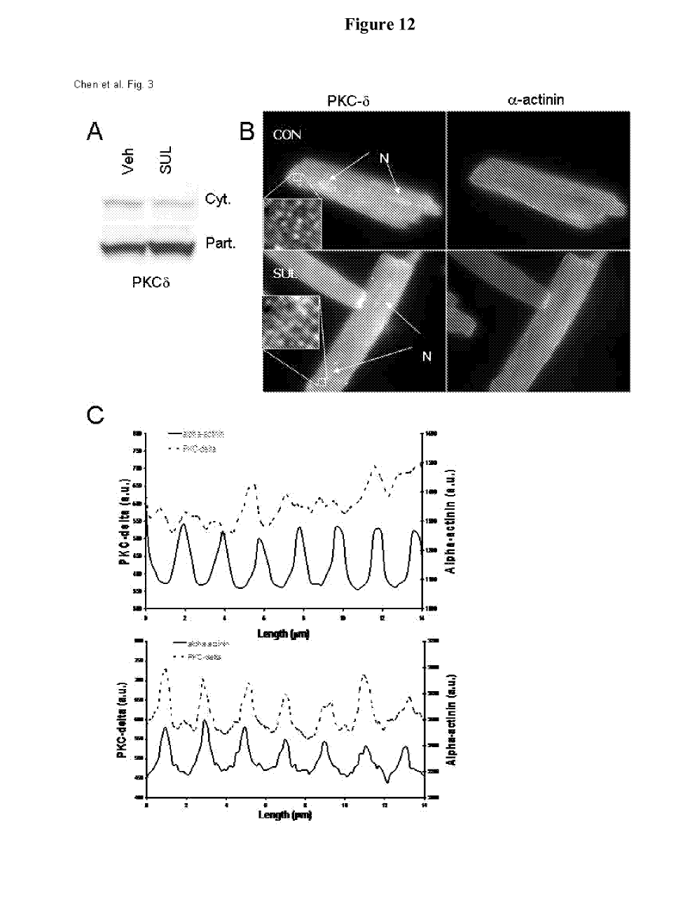

SUL triggers redistribution of PKC delta in the perfused heart and in adult rat myocytes. Cardioprotection is associated with signaling through PKC (2, 10, 11, 17, 22). PKC activation is typically accompanied by translocation from the cytosol to a membrane compartment. To determine if SUL could activate PKC, we sought evidence for redistribution of PKC delta and epsilon after SUL administration. SUL infusion into the Langendorff-perfused heart resulted in translocation of PKC delta to the particulate fraction (FIG. 12A). PKC epsilon did not show a consistent pattern of translocation (data not shown). Additionally, we studied the effect of SUL on PKC distribution in isolated adult rat cardiomyocytes. Immunostaining for PKC delta revealed a somewhat random punctate pattern under resting conditions, but after SUL administration, the distribution of PKC delta was much more closely aligned with alpha-actinin (FIG. 12B), which was further verified using pseudo-line scanning (FIG. 12C). A similar analysis for PKC epsilon did not yield a clear pattern of distribution or a detectable change in response to SUL administration (data not shown).

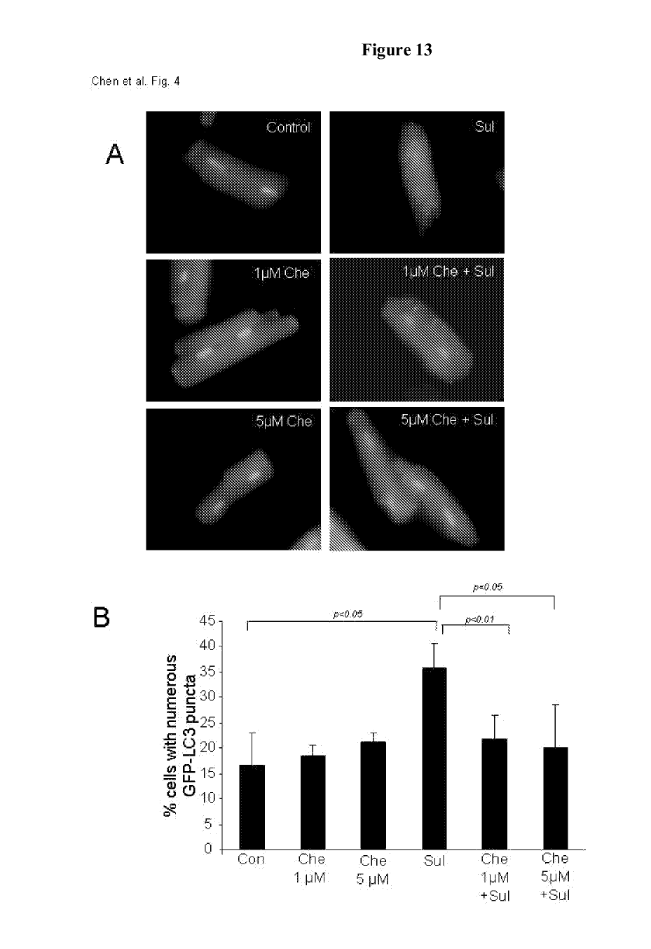

PKC mediates the induction of autophagy triggered by SUL in adult rat myocytes. To determine if PKC signaling is required for the induction of autophagy by SUL, we examined adult cardiomyocytes infected with GFP-LC3 adenovirus and treated with 10 μM SUL for 30 min. SUL significantly increased the percentage of cells with numerous autophagosomes, which was suppressed by the PKC inhibitor, chelerythrine (Che, in the figure) (FIGS. 13A and 13B).

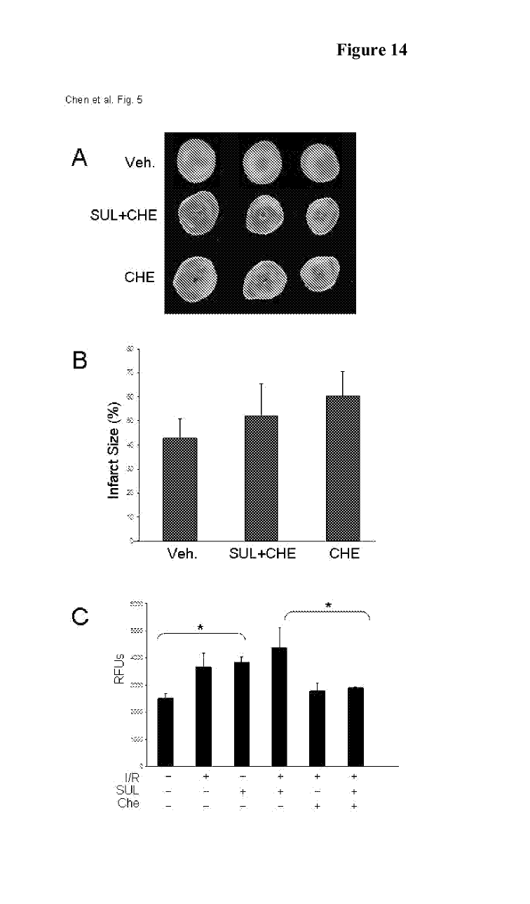

Cardioprotection and autophagy induction by SUL depends upon PKC. To determine whether PKC signaling is required for cardioprotection mediated by SUL in the ex vivo heart, we evaluated the effect of chelerythrine on infarct size in hearts treated with SUL. As shown in FIG. 14A and 14B, in the presence of chelerythrine, there is no difference in infarct size whether SUL is present or absent, indicating that cardioprotection by SUL has been established. To measure autophagy in these same tissues, we used a fluorescent conjugate of cadaverine, which incorporates into autophagosomes (33) and serves as an accurate reporter of autophagy in heart tissue (24, 38). Chelerythrine suppressed autophagy induced by SUL (FIG. 14C). These results suggest that PKC is required for the induction of autophagy and cardioprotection by SUL.

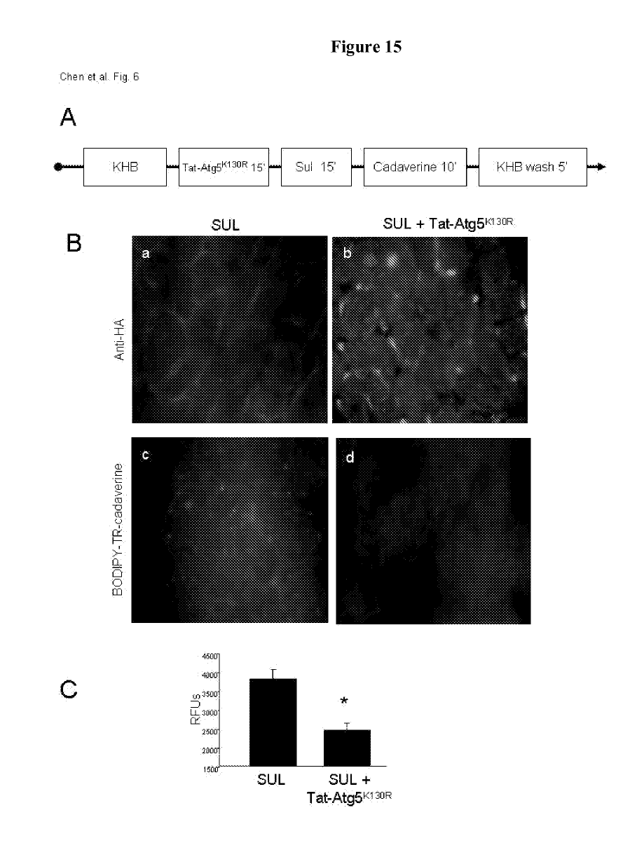

Tat-Atg5^{K130R} blocks autophagy induced by SUL in isolated perfused hearts. Atg5^{K130R} is a point mutant of Atg5 which functions as a dominant negative to inhibit autophagosome formation (20, 39). We expressed Atg5^{K130R} as a fusion protein with the protein transduction domain derived from HIV Tat (Tat-Atg5^{K130R}), and used this reagent to inhibit autophagy. We perfused rat hearts with Tat-Atg5^{K130R} and assessed its ability to block autophagy induced by SUL. For these studies, rat hearts were perfused with Tat-Atg5^{K130R} followed by SUL (FIG. 15A). We confirmed uptake of Tat-Atg5^{K130R} into cardiomyocytes by immunostaining for the hemagglutinin epitope incorporated into the Tat fusion protein (FIG. 15B, panels a, b). To measure autophagy, we used the cadaverine binding assay (FIG. 15B panels c, d, and quantified in 6C).

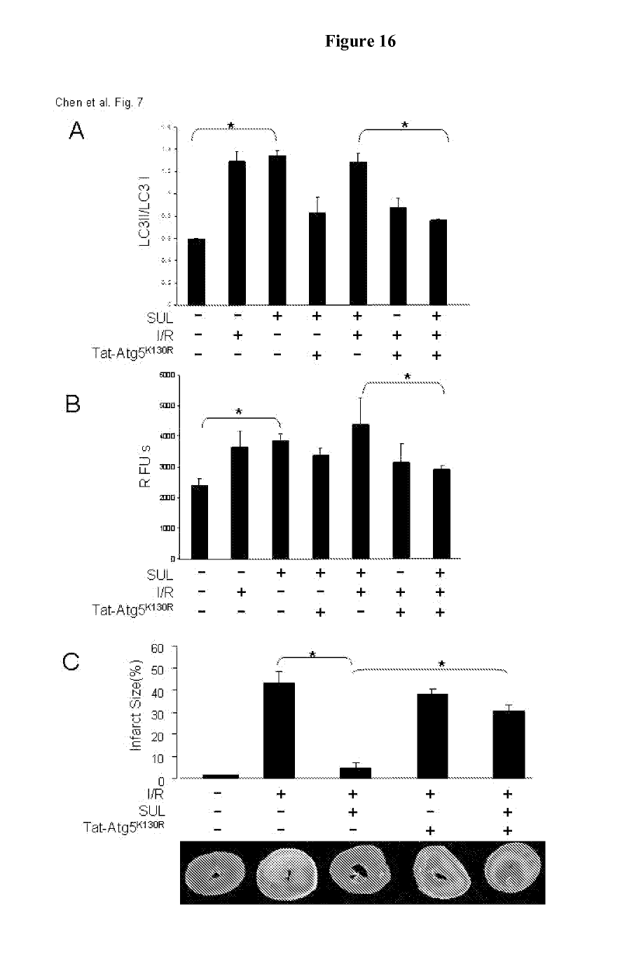

We further characterized autophagy in the setting of SUL administration and I/R, and assessed the effects of Tat-Atg5^{K130R} using immunoblotting of LC3 and cadaverine dye binding assays (FIG. 16A, B). Results were similar using LC3-II/I ratios or cadaverine dye binding, thus further validating this method to measure autophagy. These results also show that Tat-Atg5^{K130R} potently suppressed autophagy induced by SUL in the isolated perfused heart.

Tat-Atg5^{K130R} blocks cardioprotection induced by SUL in isolated perfused hearts. In order to determine if autophagy was required for cardioprotection, we perfused rat hearts with Tat-Atg5^{K130R} and assessed its effect on cardioprotection by SUL (FIG. 16C). Whereas administration of SUL reduced infarct size to 5% of the area at risk, pretreatment with Tat-Atg5^{K130R} reduced the protection afforded by SUL infusion, resulting in an infarct size of 30% of the area at risk. The fact that cardioprotection is only partially eliminated may be due to incomplete suppression of autophagy by Tat-Atg^{K130R} or to additional cardioprotective mechanisms that are independent of autophagy. In the absence of SUL, Tat-Atg5^{K130R} did not alter infarct size relative to the vehicle control (42.0% vs. 38.5%, p=NS). These results demonstrate that autophagy is required for SUL-mediated cardioprotection against I/R injury. Moreover, these results show that the exemplary Tat-Atg5^{K130R} molecule of this invention can be delivered in vivo, e.g., to an organ, and can inhibit autophagy.

### Discussion

The results of this study extend our previous finding that SUL is cardioprotective, which has subsequently been confirmed by other groups (23, 26, 27). Here, we show that SUL induced autophagy and is dependent upon signaling through PKC. The connection between SUL, PKC and autophagy is novel. Protein kinase C has been demonstrated to be essential for preconditioning, although controversy exists over which isozyme is responsible for the protective signal. For instance, preconditioning exacerbated I/R injury in PKC delta null mice (32). On the other hand, most studies have implicated PKC epsilon in cardioprotection (5, 7, 40). Our studies suggest a link between SUL and PKC delta.

Several groups have linked autophagy to cardioprotection mediated by preconditioning (18, 37, 44, 45). Effective autophagy depends upon efficient fusion of autophagosomes with functional lysosomes, which in turn requires lysosomal acidification accomplished by the vacuolar proton ATPase (VPATPase). We previously reported that inhibition of the VPATPase with bafilomycin A1 abolishes ischemic preconditioning (13, 25). Other investigators have confirmed that bafilomycin A1 blocks preconditioning (28, 41). Both PCK and PKA have been reported to trigger phosphorylation of a regulatory subunit of the VPATPase (34, 36, 42). The V-ATPase is required for lysosomal acidification, a prerequisite for autophagosome-lysosome fusion, and is therefore a critical factor in regulating autophagic flux.

A number of observations link SUL and cytochrome P450 inhibition to cardioprotective signaling. Shimamoto's group showed that SUL inhibited a cytochrome P450 activity in rat heart microsomes (23). The SUL-sensitive CYP enzyme might participate in arachidonic acid (AA) metabolism. Since AA can activate some PKC isozymes (29), inhibition of CYP-dependent conversion of AA to other products could increase AA levels and support PKC activation. AA can also be metabolized by a lipoxygenase to a cardioprotective product, so inhibiting CYPs that consume AA might increase the availability of AA to a cardioprotective lipoxygenase (9). Furthermore, the AA metabolite 20-HETE increases after I/R, and inhibition of CYPs that metabolize AA to 20-HETE is cardioprotective (35). Interestingly, 20-HETE is an inhibitor of AMPK 43). AMPK is known to induces autophagy but would be inhibited by 20-HETE. Preventing the CYP-dependent formation of 20-HETE would therefore allow AMPK to activate autophagy and achieve cardioprotection. Thus there are a number of possible links between SUL, CYP inhibition, and myocardial protection.

We have shown that SUL induces autophagy, and that autophagy is required for its cardioprotective effect. We also observed an increase in autophagy after I/R; however, based on our previously published studies of autophagic flux in HL-1 cells (19), we suspect that this is due to impaired clearance of autophagosomes rather than increased autophagosome formation. We used the exemplary cell-permeable Tat-Atg5^{K130R} to block autophagy, and observed an increase in infarct size in hearts concurrently treated with SUL. The ability to deliver the exemplary Tat-Atg5K130R to an isolated perfused heart demonstrates that it can be delivered in vivo to animals or humans; thus, in one embodiment, the invention provides compositions and methods for delivering the exemplary Tat-Atg5K130R molecule of the invention in vivo (e.g., to a heart) to animals or humans.

We did not see an increase in infarct size in hearts subjected to I/R and Tat-Atg5^{K130R}. It is possible that the heart does not mount an effect autophagic response in the absence of preconditioning, or that infarct size measurements above 50% of the area at risk are not linearly related to the extent of injury. Decreased infarct size was observed in Beclin 1 (+/−) mice subjected to 20 min ischemia and 24 hr reperfusion (30). It is possible that defective autophagy during reperfusion contributes to cell injury and inflammation, in which case less autophagy might be preferable to frustrated autophagy in vivo. Our studies in the Langendorff system do not shed light on this possibility. More work is needed to assess the role of autophagy in the context of long-term functional recovery and remodeling.

Our results with SUL clearly demonstrate a protective role for autophagy in the acute setting. It has been suggested that autophagy may be beneficial during ischemia by providing metabolic substrates (31). However, SUL is also effective when administered at reperfusion (FIG. 1), which suggests that induction of autophagy during reperfusion is sufficient. It will be important to verify these findings in an in vivo model. In addition to the generation of metabolic substrates, activation of autophagy and the VPATPase can serve as a sink for protons, thereby limiting Na⁺/H⁺ exchange and preventing Ca⁺² overload (25). Autophagy may also be important for removing damaged mitochondria which might otherwise trigger cell death. Alternatively, the amino acids generated in the autophagolysome may provide the driving force for glutathione resynthesis, thereby supporting repair of oxidized protein sulfhydryls. Regardless of the mechanism by which autophagy protects the heart subjected to I/R, the findings indicate that PKC signaling and autophagy are linked to SUL-mediated cardioprotection.

These findings reveal that SUL induces autophagy in adult rat cardiomyocytes, isolated perfused rat hearts, and intact mouse hearts. Stimulation of autophagy by SUL is mediated by a PKC-dependent pathway. The results obtained with the selective autophagy inhibitor, Tat-Atg5^{K130R}, indicate that autophagy is an important element of cardioprotective in the setting of ischemia/reperfusion injury. Given that other cardioprotective interventions such as ischemic preconditioning and an adenosine A1 agonist also induce autophagy, it is reasonable to infer that autophagy represents a common process utilized by cardiomyocytes to withstand ischemia/reperfusion injury (12, 44, 45). Induction of autophagy may represent a new therapeutic approach to myocardial protection in humans.

### Figure Legends

FIG. 10. Effects of SUL on I/R injury in isolated perfused rat hearts. A. Sulfaphenazole or vehicle was infused before 30 min of global no-flow ischemia, and coronary effluent was collected for the first 15 min of reperfusion for determination of CK release. Mean and S.D. from at least first hearts per condition are shown (* p<0.05). B. Hearts treated as above were reperfused for 120 min and infarct size was measured by TTC staining. C. Representative slices of TTC-stained hearts are shown. D-F. Preischemic SUL administration enhances recovery of function, as measured by recovery of developed pressure, dp/dtₘₐₓ, and dp/dtₘᵢₙ. Mean and S.D. from at least five hearts per condition are shown (* p<0.01, * p<0.05).

FIG. 11. SUL induces autophagy in rat and mouse hearts. A. Rat hearts were perfused with vehicle or SUL for 30 min, and then fixed and immunostained fro LC3 antibody [(a) and (b)]. Vehicle or SUL was administered by i.p. injection to mCherry-LC3 transgenic mice and hearts were removed for tissue processing 60 min later [(c) and (d)]. B. Representative Western blot to detect LC3-I and LC3-II in rat hearts perfused with vehicle or SUL. C. Quantification of LC3-II/LC3-I. Experiments were repeated 4 times (* p<0.05). D. Quantification of autophagosomes (mCherry-LC3 puncta) in hearts of mice that received vehicle or SUL (* p<0.01, n=6).

FIG. 12. Effect of SUL on PKC δ translocation. A. Immunoblots of cytosol and particulate fractions of rat hearts 30 min after SUL infusion (Langendorff). PKC δ increased in the particulate fraction and decreased in the cytosol. This blot is representative of 3 similar results. B. Fluorescence micrograph of adult rat cardiomyocytes treated with SUL or vehicle (CON) for 15 min, then fixed and immunostained with antibody to PKC δ and α-actinin. Inset shows a higher resolution field. N=nuclei. C. Pseudo-line scan derived from the myocytes shown in B, in which the fluorescence intensity (y axis; a.u., arbitrary units) is measured along a defined segment of the myocyte on the longitudinal axis (x axis). Solid line denotes the fluorescence intensity obtained with antibody to α-actinin, while the dotted line denotes the signal from antibody to PKC δ on the same segment. The increased regularity of PKC δ distribution (co-localization with α-actinin) after SUL administration was a consistent finding (N=3). PKC δ distribution coincided with Z-lines, which may be consistent with association with T-tubules.

FIG. 13. Role of PKC in autophagy induction by SUL in rat cardiomyocytes. A. Isolated adult cardiomyocytes were infected with GFP-LC3 adenovirus. The next day, cells were treated with SUL with or without the PKC inhibitor, chelerythrine (Che). Autophagy is induced by SUL in adult rat cardiomyocytes but is suppressed by chelerythrine. B. Quantification of autophagy by percentage of cells displaying numerous puncta. Experiments were repeated 3 times.

FIG. 14. Role of PKC in autophagy and cardioprotection in isolated perfused rat hearts. A. Hearts were treated with chelerythrine with or without SUL, then subjected to I/R and stained with TTC for infarct size determination. B. Quantification of infarct size after administration of chelerythrine is shown (p=NS, n=4). C. Quantification of autophagy in perfused hearts treated as indicated and measured by cadaverine dye binding assay (*p<0.03, n=3).

FIG. 15. Effects of Tat-Atg5^{K130R} and SUL on autophagy in isolated perfused rat hearts:

FIG. 15A. Protocol for Langendorff perfusion. Rat hearts were stabilized for 15 min, followed by treatments as indicated.

FIG. 15B. Tat-Atg5^{K130R} in cardiomyocytes is detected by anti-HA antibody (green immunofluorescence). This shows that the Tat protein (the exemplary Tat-Atg5^{K130R} molecule) was successfully delivered into the heart and taken up by cardiomyocytes.

BODIPY-TR™-cadaverine incorporation into autophagosomes (red fluorescence) was increased by SUL administration (reflecting increased autophagy) and diminished by pre-treatment with Tat-Atg5^{K130R}. This shows that the exemplary Tat-Atg5^{K130R} molecule blocked autophagy.

FIG. 15C. Quantification of autophagy by cadaverine dye binding in heart tissue (p<0.005).). The reduction in dye binding in the exemplary Tat-Atg5^{K130R} protein perfused heart indicates that it suppressed autophagy.

FIG. 16. Induction of autophagy by SUL is abolished by administration of Tat-Atg5^{K130R}. Rat hearts were perfused with Tat-Atg5^{K130R} as indicated in FIG. 6 followed by addition of SUL or vehicle to perfusion buffer and treatment as indicated. A. Quantification of the LC3-II/LC3-I ratio from Western blots (*p<0.01, N=3). B. Quantification of autophagy by cadaverine binding assay (*p<0.02, N=6). C. Hearts treated as above were reperfused for 120 min and infarct size was determined by TTC staining. Shown are quantification of infarct size (*p<0.01, N=5) and representative TTC-stained heart sections. This verifies a second downstream functional consequence of inhibiting autophagy and provides further evidence that the exemplary Tat-Atg5K130R molecule can be delivered to an organ to inhibit autophagy.

### REFERENCES

1. Alessi D R, Andjelkovic M, Caudwell B, Cron P, Morrice N, Cohen P, and Hemmings B A. Mechanism of activation of protein kinase B by insulin and IGF-1. EMBO J 15:6541-6551, 1996.

2. Armstrong S, Downey J M, and Ganote C E. Preconditioning of isolated rabbit cardiomyocytes: induction by metabolic stress and blockade by the adenosine antagonist SPT and calphostin C, a protein kinase C inhibitor. Cardiovascular Research 28:72-77, 1994.

3. Arnaud C, Joyeus-Faure M, Bottari S, Godin-Ribuot D, and Ribuot C. New insight into the signaling pathways of heat stress-induced myocardial preconditioning: protein kinase Cepsilon translocation and heat shock protein 27 phosphorylation. ClinExp Pharmacol Physiol 31:129-133, 2004.

4. Becker-Hapak M, McAllister S S, and Dowdy S F. TAT-Mediated Protein Transduction into Mammalian Cells, Methods 24:247-256, 2001.

5. Budas G R, Churchill E N, and Mochly-Rosen D. Cardioprotective mechanisms of PKC isozyme-selective activators and inhibitors in the treatment of ischemia-reperfusion injury. Pharmacol Res 55:523-536, 2007.

6. Caro L H P, Plomp P J A M, Wolvetang E J, Kerkof C, and Meijer A J. 3-Methyladenine, and inhibitor of autophagy, has multiple effects on metabolism. European Journal of Biochemistry 175:325-329, 1988.

7. Chen L, Hahn H, Wu G, Chen C H, Liron T, Schechtman D, Cacallaro G, Banci L, Guo Y, Bolli R, Dorn G W, 2nd, and Mochly-Rosen D. Opposing cardioprotective actions and parallel hypertrophic effects of delta PKC and epsilon PKC. Proc Natl Acad Sci U S A 98:11114-11119, 2001.

8. Chen M, Won D J, Krajewski S, and Gottlieb R A. Calpain and mitochondria in ischemia/reperfusion injury. J Biol Chem 277:29181-29186, 2002.

9. Chen W. Glasgow W, Murphy E, and Steenbergen C. Lipxygenase metabolism of arachidonic acid in ischemic preconditioning and PKC-induced protection in heart. Am J Physiol 276:H2094-H2101, 1999.

10. Das A, Ockaili R, Salloum F, and Kukreja R C. Protein kinase C plays an essential role in sildenafil-induced cardioprotection in rabbits. AJP—Heart and Circulatory Physiology 286:H1455-H1460, 2004.

11. Fryer R M, Wang Y, Hsu A K, and Gross G J. Essential activation of PKC-delta in opioid-initiated cardioprotection. AJP—Heart and Circulatory Physiology 280:H1346-H1353, 2001.

12. Gottlieb R A, Finley K D, and Mentzer R M, Jr. Cardioprotection requires taking out the trash. Basic Res Cardiol 104:169-180, 2009.

13. Gottlieb R A, Gruol D L, Zhu J Y, and Engler R L. Preconditioning in rabbit cardiomyocytes: Role of pH, vacuolar proton ATPase, and apoptosis. Journal of Clinical Investigation 97:2391-2398, 1996.

14. Granfeldt A, Lefer D J, and Vinten-Johansen J. Protection ischaemia in patients: preconditioning and postconditioning. Cardiovasc Res 83:234-246, 2009.

15. Granville D J, Tashakkor B, Takeuchi C, Gustafsson A B, Huang C, Sayen M R, Wentworth P, Jr., Yeager M, and Gottlieb R A. Reduction of ischemia and reperfusion-induced myocardial damage by cytochrome P450 inhibitors. ProcNatlAcadSci USA 101:1321-1326, 2004.

16. Granville D J, Tashakkor B, Takeuchi C, Gustafsson A B, Huang C, Sayen M R, Wentworth P, Jr., Yeager M, and Gottlieb R A. Reduction of ischemia and reperfusion-induced myocardial damage by cytochrome P450 inhibitors. Proc Natl Acad Sci U S A 101:1321-1326, 2004.

17. Gray M O, Zhou H Z, Schafhalter-Zoppoth I, Zhu P, Mochly-Rosen D, and Messing R O. Preservation of base-line hemohynamic function and loss of inducible cardioprotection in adult mice lacking protein kinase C epsilon. J BiolChem 279:3596-3604, 2004.

18. Gurusamy n, Lekli I, Gherghiceanu M, Popescu L M, and Das DK. BAG-1 induces autophagy for cardiac cell survival. Autophagy 5:120-121, 2009.

19. Hamacher-Brady A, Brady N R, and Gottlieb R A. Enhancing macroautophagy protects against ischemia/reperfusion injury in cardiac myocytes. J Biol Chem 281:29776-29787, 2006.

20. Hamacher-Brady A, Brady N R, Logue S E, Sayen M R, Jinno M, Kirshenbaum L A, Gottlieb R A, and Gustafsson A B. Response to myocardial ischemia/reperfusion injury involves Bnip3 and autophagy. Cell Death Differ 14:146-157, 2007.

21. He H, Li H L, Lin A, and Gottlieb R A. Activation of the JNK pathway is important for cardiomyocyte death in response to simulated ischemia. Cell Death Differ 6:987-991, 1999.

22. Hirotani S and Sadoshima J. Preconditioning effects of PKC[delta]. Journal of Molecular and Cellular Cardiology 39:719-721, 2005.

23. Ishihara Y, Sekine M, Nakazawa M, and Shimamoto N. Suppression of myocardial ischemia-reperfusion injury by inhibitors of cytochrome P450 in rats. Eur J Pharmacol 611:64-71, 2009.

24. Iwai-Kanai E, Yuan H, Huang C, Sayen M R, Perry-Garza C N, Kim L, and Gottlieb R A. A method to measure cardiac autophagic flux in vivo. Autophagy 4:322-329, 2008.

25. Karwatowska-Prokopezuk E, Nordberg J, Li H L, Engler R L, and Gottlieb R A. Effect of the vacuolar proton ATPase on intracellular pH, calcium, and on apoptosis in neonatal cardiomyocytes during metabolic inhibition and recovery. CircRes 82:1139-1144, 1998.

26. Khan M, Iyyapu K M, Kutala V, Kotha S, Parinandi N L, Hamlin R L, and Kuppusamy P. Sulfaphenazole protects heart against ischemia-reperfusion injury and cardiac dysfunction by overexpression of iNOS leading to enhancement of nitric-oxide bioavailability and tissue oxygenation. Antioxid Redox Signal, 2008.

27. Khan M, Mohan I K, Kutala V K, Kumbala D, and Kuppusamy P. Cardioprotection by sulfaphenazole, a cytochrome p450 inhibitor: mitigation of ischemia-reperfusion injury by scavenging of reactive oxygen species. J Pharmacol Exp Ther 323:813-821, 2007.

28. Long X, Crow M T, Sollott S J, O'Neill K L, Menees D S, Hipolito L, Boluyt M O, Asai T, and Lakatta E G. Enhanced expression of p53 and apoptosis induced by blockade of the vacuolar proton ATPase in cardiomyocytes. J Clin Invest 101:1453-1461, 1998.

29. Mackay K and Mochly-Rosen D. Arachidonic acid protects neonatal rat cardiac myocytes from ischaemic injury through epsilon protein kinase C. Cardiovasc Res 50:65-74, 2001.

30. Matsui Y, Takagi H, Qu X, Abdellatif M, Sakoda H, Asano T, Levine B, and Sadoshima J. Distinct roles of autophagy in the heart during ischemia and reperfusion: roles of AMP-activated protein kinase and Beclin 1 in mediating autophagy. Circ Res 100:914-922, 2007.

31. Matsui Y, Takagi H, Qu X, Abdellatif M, Sakoda H, Asano T, Levine B, and Sadoshima J. Distinct Roles of Autophagy in the Heart During Ischemia and Reperfusion Roles of AMP-Activated Protein Kinase and Beclin 1 in Mediating Autophagy. Circ Res 100:914-922, 2007.

32. Mayr M, Metzler B, Chung Y L, McGregor E, Mayr U, Troy H, Hu Y, Leitges M, Pachinger O, Griffiths J R, Dunn M J, and Xu Q. Ischemic preconditioning exaggerates cardiac damage in PKC-delta null mice. Am J Physiol Heart Circ Physiol 287:H946-956, 2004.

33. Munafo D B and Colombo M I. A novel assay to study autophagy: regulation of autophagosome vacuole size by amino acid deprivation. J Cell Sci 114:3619-3629, 2001.

34. Nanda A, Gukovskaya A, Tseng J, and Grinstein S. Activation of vacuolar-type proton pumps by protein kinase C. Role in neutrophil pH regulation. Journal of Biological Chemistry 267:22740-22746, 1992.

35. Nithipatikom K, Gross E R, Endsley M P, Moore J M, Isbell M A, Falck J R, Campbell W B, and Gross G J. Inhibition of Cytochrome P450 {omega}-Hydroxylase: A Novel Endogenous Cardioprotective Pathway. Circulation Research 95:e65-e71, 2004.

36. Nordstrom T, Grinstein S, Brisseau G F, Manolson M F, and Rotstein O D. Protein kinase C activation accelerates proton extrusion by vacuolar-type H(+)-ATPases in murine peritoneal macrophages. FEBS Lett 350:82-86, 1994.

37. Park H K, Chu K, Jung K H, Lee S T, Bahn J J, Kim M, Lee S K, and Roh J K. Autophagy is involved in the ischemic preconditioning. Neurosci Lett 451:16-19, 2009.

38. Perr C N, Kyoi S, Hariharan N, Takagi H, Sadoshima J, and Gottlieb R A. Novel methods for measuring cardiac autophagy in vivo. Methods Enzymol 453:325-342, 2009.

39. Pyo-J-O, Jang M-H, Kwon Y-K, Lee H-J, Jun J-IL, Woo H-N, Cho D-H, Choi B, Lee H, Kim J-H, Mizushima N, Oshumi Y, and Jung Y-K. Essential Roles of Atg5 and FADD in Autophagic Cell Death: Dissection of Autophagic Cell Death into Vacuole Formation and Cell Death. J Biol Chem 280:20722-20729, 2005.

40. Sivaraman V, Hausenloy D J, Kolvekar S, Hayward M, Yap J, Lawrence D, Di Salvo c, and Yellon D M. The divergent roles of protein kinase C epsilon and delta in simulated ischaemia-reperfusion injury in human myocardium. J Mol Cell Cardio 46: 758-764, 2009.

41. Tong H, Rockman H A, Koch W J, Steenbergen C, and Murphy E. G protein-coupled receptor internalization signaling is required for cardioprotection in ischemic preconditioning, Circ Res 94:1133-1141, 2004.

42. Voss M, Vitavska O, Walz B, Wieczorek H, and Baumann O. Stimulus-induced phosphorylation of vacuolar H(+)-ATPas by protein kinase A. J Biol Chem 282:33735-33742, 2007.

43. Ward N, Chen K, Croft K, and Keaney J, Jr. Abstract 224:20-hydroxyeicosatetraenoic Acid Mediated Amp Activated Protein Kinase Suppression Induces Endothelial Nitric Oxide Synthase Uncoupling. Circulation 118:S_{—}274-b-, 2008.

44. Yan L, Sadoshima J, Vatner D E, and Vatner S F. Autophagy in ischemic preconditioning and hibernating myocardium. Autophagy 5:709-712, 2009.

45. Yitzhaki S, Huang C, Liu W, Lee Y, Gustafsson A B, Mentzer R M, Jr., and Gottlieb R A. Autophagy is required for preconditioning by the adenosine A1 receptor-selective agonist CCPA. Basic Res Cardiol 104:157-167, 2009.

46. Yuan H, Perry C N, Huang C, Iwai-Kanai E, Carreira R S, Glembotski C C, and Gottlieb R A. LPS-Induced Autophagy Is Mediated by Oxidative Signaling in Cardiomyocytes and is Associated with Cytoprotection. Am J Physiol Heart Cire Physiol, 2008.

### Molecular Perturbation of Autophagy

During formation of the pre-autophagosomal structure, the C-terminal glycine of Atg12 forms a bond with Atg5 lysine 130. Replacing Atg5 lysine 130 with arginine (Atg5^{K130R}) renders Atg5 unable to accept Atg12, and thus blocks AV formation, including LC3 recruitment. In order to enable molecular perturbation of the autophagic pathway, we generated and characterized fusion proteins of the monomeric red fluorescent protein mCherry and Atg5 or the dominant negative mutant of Atg5, Atg5^{K130R}.

We previously demonstrated that expression of mCherry-Atg5 did not significantly influence autophagic flux in either high or low nutrient conditions when compared to control (mCherry-expressing) cells, but that expression of the mutant mCherry-Atg5^{K130R} significantly reduced both steady-state and lysosomal inhibitor-sensitive accumulation of AVs in response to simulated I/R or Bnip3 overexpression. GFP-LC3-labeled puncta were smaller in mCherry-Atg^{K130R} cells than in control or mCherry-Atg5 cells, indicative of failed pre-autophagosome maturation.

In order to study autophagy ex vivo/in vivo, we prepared recombinant TAT-Atg5^{K130R} and perfused it into rat hearts in the Langendorff model. This reagent potently suppressed autophagy, and importantly, it blocked the cardioprotective effects of sulfaphenazole, demonstrating that autophagy is required for protection in the sulfaphenazole-treated heart subjected to I/R (FIG. 8). This important result needs additional verification, and the method will be applied to other conditioning agents such as adenosine agonists, as outlined in Aim One.

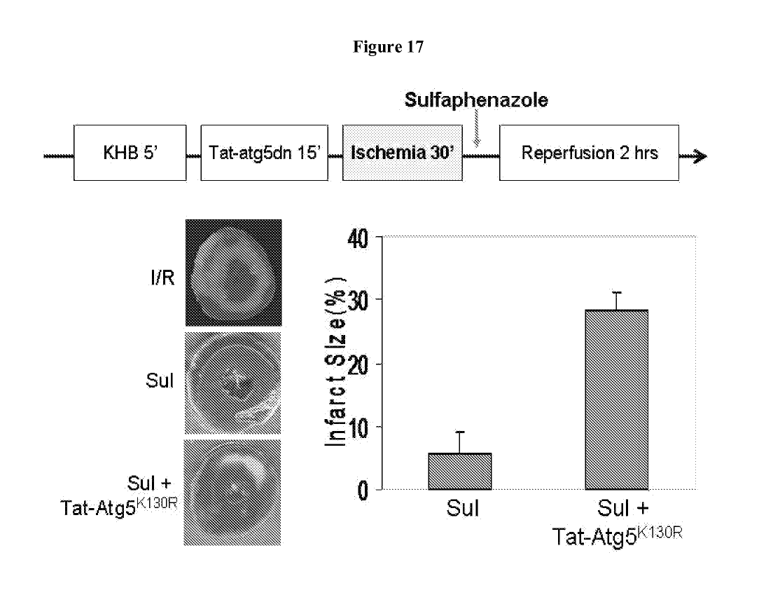

FIG. 17 illustrates that sulfaphenazole (Sul) reduces infarct size when given at reperfusion, but the protection is lost if autophagy is blocked with Tat-Atg5^{K130R}. Representative TTC-stained sections are shown, and quantitation is based on 3 hearts per condition.

We have previously shown that overexpression of Beclin1 is sufficient to increase autophagy and to protect HL-1 cells against simulated I/R injury. We have been able to express and purify recombinant Tat-Beclin1 and to demonstrate protection in cell culture (FIG. 9). These reagents as well as the fluorescent cadaverine reagents can be used to monitor and perturb autophagy.

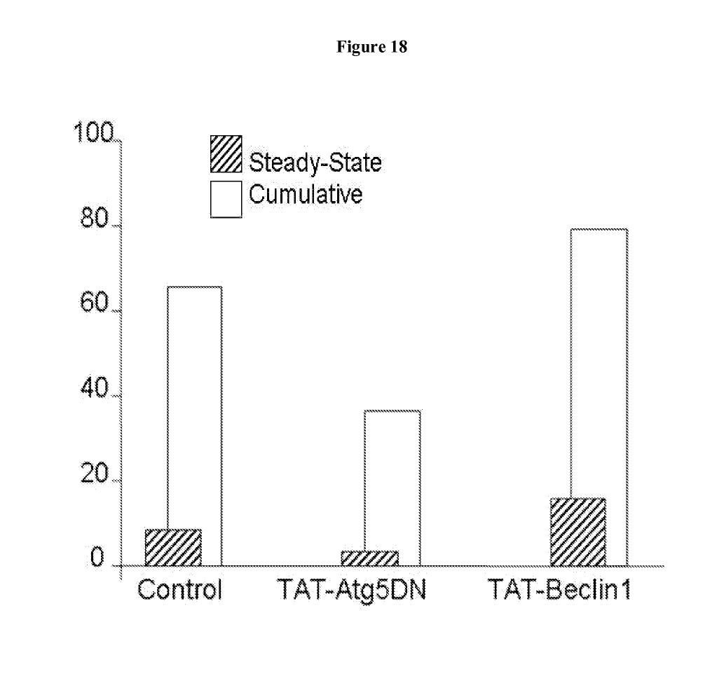

FIG. 18 illustrates that Tat proteins can modulate autophagy. HL-1 cells were transfected with LC3GFP and then treated with Tat-Atg5^{K130R} (which inhibits autophagy) or Tat-Beclin1 (which stimulates autophagy).

A number of embodiments of the invention have been described. Nevertheless, it will be understood that various modifications may be made without departing from the spirit and scope of the invention. Accordingly, other embodiments are within the scope of the following claims.

1. An isolated, recombinant or synthetic nucleic acid encoding a chimeric (hybrid) protein, wherein the chimeric (hybrid) protein comprises:
(i) a first domain comprising or consisting of: a peptide and/or a small molecule that confers cell permeability, a protein transduction domain of an HIV Tat protein, the 11 amino acid protein transduction domain of HIV Tat; the protein transduction domain of Antennapedia; the Drosophila homeoprotein antennapedia transcription protein (AntHD); a poly-arginine sequence; a cationic N-terminal domain of a prion protein; a herpes simplex virus structural protein VP22; peptidomimetics and synthetic forms thereof; and, all equivalents and variants thereof capable of acting as a protein transduction domain, and
(ii) a second domain comprising or consisting of: a sequence comprising all or a subsequence of a wild type (non-mutated or manipulated) Atg5, or SEQ ID NO:7; a sequence comprising all or a subsequence of an Atg5 with its lysine 130 mutated to an arginine or another (non-lysine) amino acid; a sequence comprising all or a subsequence of Beclin 1;
wherein optionally the protein comprises or consists of a Tat-Atg5K130R (Tat-Atg5^{K130R}) (inhibitor of autophagy), a Tat-Beclin 1 (stimulates or increases autophagy), or a peptidomimetic or synthetic form thereof, or an equivalent thereof.

2. A vector, recombinant virus, cloning vehicle, expression cassette, cosmid or plasmid comprising or having contained therein the isolated, recombinant or synthetic nucleic acid of claim 1.

3. A chimeric or hybrid polypeptide comprising (or consisting of): (a) the polypeptide encoded by the nucleic acid of claim 1; or (b) the chimeric (hybrid) protein of (a), wherein the protein comprises a synthetic protein or peptide, recombinant protein or peptide, a peptidomimetic or a combination thereof.

4. A chimeric or hybrid protein comprising:
(a) (i) a first domain comprising or consisting of: a peptide and/or a small molecule that confers cell permeability, a protein transduction domain of an HIV Tat protein, the 11 amino acid protein transduction domain of HIV Tat; the protein transduction domain of Antennapedia; the Drosophila homeoprotein antennapedia transcription protein (AntHD); a poly-arginine sequence; a cationic N-terminal domain of a prion protein; peptidomimetics and synthetic forms thereof; and, all equivalents and variants thereof capable of acting as a protein transduction domain, and
(ii) a second domain comprising or consisting of: a sequence comprising all or a subsequence of a wild type (non-mutated or manipulated) Atg5, or SEQ ID NO:7; a sequence comprising all or a subsequence of an Atg5 with its lysine 130 mutated to an arginine or another (non-lysine) amino acid; a sequence comprising all or a subsequence of Beclin 1, e.g., a Beclin 1 fragment lacking the Bcl-2 binding domain such that it inhibits autophagy, or a peptidomimetic or synthetic form thereof, or an equivalent thereof;
wherein optionally the protein comprises or consists of a Tat-Atg5K130R (Tat-Atg5^{K130R}) (inhibitor of autophagy), a Tat-Beclin 1 (stimulates or increases autophagy), or a peptidomimetic or synthetic form thereof, or an equivalent thereof;
(b) the chimeric (hybrid) protein of (a), further comprising a tag or detection moiety, or an antibody or an antigen binding fragment thereof; or
(c) the chimeric (hybrid) protein of (a) of (b), wherein the protein comprises (or consists of) a synthetic protein or peptide, recombinant protein or peptide, a peptidomimetic or a combination thereof.

5. A cell comprising the isolated, recombinant or synthetic nucleic acid of claim 1; wherein optionally the cell is a mammalian or a human cell.

6. A pharmaceutical composition or a formulation comprising the chimeric or hybrid protein of claim.

7. A method for modulating autophagy in a cell, comprising:
(a) providing: (i) a nucleic acid of claim 1, operatively linked to a transcriptional regulatory unit, or (ii) a vector, recombinant virus, cloning vehicle, expression cassette, cosmid or plasmid comprising the nucleic acid of claim 1; and, a cell comprising an environment capable of supporting the expression of the chimeric (hybrid) protein by the nucleic acid; and
(b) inserting (e.g., transfecting or infecting) the nucleic acid, vector, recombinant virus, cloning vehicle, expression cassette, cosmid or plasmid of (a) into the cell.

8. The method of claim 7, wherein the transcriptional regulatory unit comprises a promoter, an inducible promoter or a constitutive promoter.

9. The method of claim 7, wherein the cell is a mammalian cell, a monkey cell or a human cell.

10. The method of claim 7, wherein the nucleic acid, vector, recombinant virus, cloning vehicle, expression cassette, cosmid or plasmid is inserted into the cell in vivo or in vitro.

11. A method for modulating autophagy in a cell, comprising:
(a) providing a chimeric or hybrid polypeptide of claim 3; and
(b) inserting (e.g., transfecting or infecting) chimeric or hybrid polypeptide of (a) into the cell.

12. The method of claim 11, wherein the cell is a mammalian cell, a monkey cell or a human cell.

13. The method of claim 11, wherein the chimeric or hybrid polypeptide is inserted into the cell in vivo or in vitro.

14. A method for ameliorating, preventing or treating a disease, a condition or a disorder responsive to an autophagy modulation comprising administering to an individual in need thereof a sufficient amount of: the pharmaceutical composition of claim 6.

15. The method of claim 13, wherein the disease, condition or disorder treated, prevented or ameliorated comprises neurodegeneration, cystic fibrosis, cancer, heart failure, diabetes, obesity, sarcopenia, aging, ischemia/reperfusion, inflammatory disorders including Crohns, ulcerative colitis, biliary cirrhosis, lysosomal storage diseases, infectious diseases associated with intracellular pathogens including viruses, bacteria, and parasites such as Trypanosomes and malaria.

16. The method of claim 14, where the autophagy is modulated in order to increase the efficacy of a vaccine.

17. A method for increasing the efficacy of a vaccine, comprising
administering to an individual in need thereof a sufficient amount of: the nucleic acid of claim 1, operatively linked to a transcriptional regulatory unit or a vector, recombinant virus, cloning vehicle, expression cassette, cosmid or plasmid comprising the nucleic acid of claim 1.

18. The nucleic acid of claim 1, wherein the encoded chimeric (hybrid) protein further comprises a tag or detection moiety, wherein optionally the tag or detection moiety comprises a tag for an antibody or an antigen binding fragment thereof (the antibody binding specifically to the tag or detection moiety, or the tag or detection moiety comprises a ligand, or the tag or detection moiety comprises a FLAG molecule or equivalent thereof.

19. The nucleic acid of claim 1, wherein the nucleic acid encoding the chimeric (hybrid) protein is operatively linked to a transcriptional regulatory unit, or a promoter such as an inducible or constitutive promoter.

20. The nucleic acid of claim 1, wherein the Beclin 1 subsequence comprises a Beclin 1 fragment lacking the Bcl-2 binding domain such that it inhibits autophagy, or a peptidomimetic or synthetic form thereof, or an equivalent thereof.


SEQUENCE LISTING (7 sequences)

SEQ ID NO: 1 - protein, 347 aa, artificial sequence
  synthetic peptide or nucleic acid
MRGSHHHHHHGMASMTGGQQMGRDLYDDDDKDRWGSKLGYGRKKRRQRRRGGSTMSGYPYDVPDYAGSMAGTMTDDKDVLRDVWFGRIPTCFTLYQDEITEREAEPYYLLLPRVSYLTLVTDKVKKHFQKVMRQEDVSEIWFEYEGTPLKWHYPIGLLFDLLASSSALPWNITVHFKSFPEKDLLHCPSKDAVEAHFMSCMREADALKHKSQVINEMQKKDHKQLWMGLQNDRFDQFWAINRKLMEYPPEENGFRYIPFRIYQTTTERPFIQKLFRPVAADGQLHTLGDLLREVCPSAVAPEDGEKRSQVMIHGIEPMLETPLQWLSEHLSYPDNFLHISIVPQPTD

SEQ ID NO: 2 - DNA, 1044 bp, artificial sequence
  synthetic nucleic acid
atgcggggttctcatcatcatcatcatcatggtatggctagcatgactggtggacagcaaatgggtcgggatctgtacgacgatgacgataaggatcgatggggatccaagcttggctacggccgcaagaaacgccgccagcgccgccgcggtggatccaccatgtccggctatccatatgacgtcccagactatgctggctccatggccggtaccatgacagatgacaaagatgtgcttcgagatgtgtggtttggacgaattccaacttgctttactctctatcaggatgagataactgaaagagaagcagaaccatactatttgcttttgccaagagtcagctatttgacgttggtaactgacaaagtgaaaaagcactttcagaaggttatgagacaagaagatgttagtgagatatggtttgaatatgaaggcacacccctgaaatggcattatccaattggtttactatttgatcttcttgcatcaagttcagctcttccttggaacatcacagtacatttcaagagttttccagaaaaggaccttctacactgtccatccaaggatgcggttgaggctcactttatgtcgtgtatgagagaagctgatgctttaaagcataaaagtcaagtgatcaacgaaatgcagaaaaaagaccacaagcagctctggatgggactgcagaatgacagatttgaccagttttgggccatcaaccggaaactcatggaatatcctccagaagaaaatggatttcgttatatcccctttagaatatatcagaccacgacggagcggcctttcatccagaagctgttccggcctgtggccgcagatggacagctgcacacacttggagatctcctcagagaagtctgtccttccgcagtcgcccctgaagatggagagaagaggagccaggtgatgattcacgggatagagccaatgctggaaacccctctgcagtggctgagcgagcatctgagctacccagataactttcttcatattagcattgtcccccagccaacagattga

SEQ ID NO: 3 - protein, 523 aa, artificial sequence
  synthetic peptide
MRGSHHHHHHGMASMTGGQQMGRDLYDDDDKDRWGSKLGYGRKKRRQRRRGGSTMSGYPYDVPDYAGSMAGTGLEMEGSKASSSTMQVSFVCQRCSQPLKLDTSFKILDRVTIQELTAPLLTTAQAKPGESQEEEANSGEEPFIETRQDGVSRRFIPPARMMSTESANSFTLIGEASDGGTMENLSRRLKVTGDLFDIMSGQTDVDHPLCEECTDTLLDQLDTQLNVTENECQNYKRCLEMLEQMNEGDSEQLQRELKELALEEERLIQELEDVEKNRKVVAENLEKVQAEAERLDQEEAQYQREYSEFKRQQLELDDELKSVENQMRYAQMQLDKLKKTNVFNATFHIWHSGQFGTINNFRLGRLPSAPVEWNEINAAWGQTVLLLHALANKMGLKFQRYRLVPYGNHSYLESLTDKSKELPLYCSGGLRFFWDNKFDHAMVAFLDCVQQFKEEVEKGETRFCLPYRMDVEKGKIEDTGGSGGSYSIKTQFNSEEQWTKALKFMLTNLKWGLAWVSSQFYNK

SEQ ID NO: 4 - DNA, 1572 bp, artificial sequence
  synthetic nucleic acid
atgcggggttctcatcatcatcatcatcatggtatggctagcatgactggtggacagcaaatgggtcgggatctgtacgacgatgacgataaggatcgatggggatccaagcttggctacggccgcaagaaacgccgccagcgccgccgcggtggatccaccatgtccggctatccatatgacgtcccagactatgctggctccatggccggtaccggtctcgagatggaggggtctaaggcgtccagcagcaccatgcaggtgagcttcgtgtgccagcgctgtagccagcctctgaaactggacacgagcttcaagatcctggaccgagtgaccattcaggaactcacagctccattacttaccacagcccaggcgaaaccaggagagagccaggaggaagaggctaactcaggagaggagccatttattgaaactcgccaggatggtgtctctcgaagattcatccccccagccaggatgatgtctacagaaagtgctaatagcttcactctgatcggggaggcatctgatggtggcaccatggagaacctcagccggagactcaaggtcactggagacctttttgacatcatgtctggccagacagatgtggatcacccactgtgtgaggaatgcacagatactcttttagaccagctggacactcagctcaatgttactgaaaacgagtgtcagaactacaaacgctgtttggagatgttggagcaaatgaatgagggcgacagtgaacagctacagagggagctgaaggagttggccttggaggaggagaggctgatccaggagctggaagatgtggaaaaaaaccgaaaggtggtggcagaaaacctggagaaggtccaggctgaggcggagagactggaccaggaggaagctcagtaccagcgagaatatagtgaatttaaaaggcagcagctggagctggatgatgagctcaagagtgtagagaaccagatgcgctatgcccagatgcagctggacaagctcaagaaaaccaatgtcttcaatgcgaccttccatatctggcacagcggacaatttggcacgatcaataatttcagactgggtcgcttgcccagtgctcctgtggaatggaatgaaatcaatgctgcctggggccagacagtgttgttgctccatgctttggccaataagatgggtctgaaatttcagaggtaccgacttgttccctatggaaatcactcgtatctggagtccctgacagacaaatctaaggagttgccgttgtactgttctgggggtttgcggtttttctgggacaacaagtttgaccatgcaatggtagcttttctggactgtgtgcagcagttcaaagaagaggtggaaaaaggagagactcgattttgtcttccgtacaggatggacgtggagaaaggcaagattgaagacactggaggcagtggcggctcctattccatcaaaacccagtttaactctgaggagcagtggacaaaggcgctcaagttcatgctgacgaatctcaagtggggtcttgcttgggtgtcctcacagttctataacaagtga

SEQ ID NO: 5 - protein, 293 aa, artificial sequence
  synthetic peptide
MRGSYGRKKRRQRRRGGSMTDDKDVLRDVWFGRIPTCFTLYQDEITEREAEPYYLLLPRVSYLTLVTDKVKKHFQKVMRQEDISEIWFEYEGTPLKWHYPIGLLFDLLASSSALPWNITVHFKSFPEKDLLHCPSKDAIEAHFMSCMREADALKHKSQVINEMQKKDHKQLWMGLQNDRFDQFWAINRKLMEYPAEENGFRYIPFRIYQTTTERPFIQKLFRPVAADGQLHTLGDLLKEVCPSAIDPEDGEKKNQVMIHGIEPMLETPLQWLSEHLSYPDNFLHISIIPQPTD

SEQ ID NO: 6 - DNA, 3244 bp, Homo sapiens
gtgacgtcatctccgggcgccgagggtgactggacttgtggtgcgctgccagggctccgcagcgttgccggttgtattcgctggataccagagggcggaagtgcagcagggttcagctccgacctccgcgccggtgctttttgcggctgcgcgggcttcctggagtcctgctaccgcgtccccgcaggacagtgtgtcaggcgggcagcttgccccgccgccccaccggagcgcggaatctgggcgtccccaccagtgcggggagccggaaggaggagccatagcttggagtaggtttggctttggttgaaataagaatttagcctgtatgtactgctttaactcctggaagaatgacagatgacaaagatgtgcttcgagatgtgtggtttggacgaattccaacttgtttcacgctatatcaggatgagataactgaaagggaagcagaaccatactatttgcttttgccaagagtaagttatttgacgttggtaactgacaaagtgaaaaagcactttcagaaggttatgagacaagaagacattagtgagatatggtttgaatatgaaggcacaccactgaaatggcattatccaattggtttgctatttgatcttcttgcatcaagttcagctcttccttggaacatcacagtacattttaagagttttccagaaaaagaccttctgcactgtccatctaaggatgcaattgaagctcattttatgtcatgtatgaaagaagctgatgctttaaaacataaaagtcaagtaatcaatgaaatgcagaaaaaagatcacaagcaactctggatgggattgcaaaatgacagatttgaccagttttgggccatcaatcggaaactcatggaatatcctgcagaagaaaatggatttcgttatatcccctttagaatatatcagacaacgactgaaagacctttcattcagaagctgtttcgtcctgtggctgcagatggacagttgcacacactaggagatctcctcaaagaagtttgtccttctgctattgatcctgaagatggggaaaaaaagaatcaagtgatgattcatggaattgagccaatgttggaaacacctctgcagtggctgagtgaacatctgagctacccggataattttcttcatattagtatcatcccacagccaacagattgaaggatcaactatttgcctgaacagaatcatccttaaatgggatttatcagagcatgtcacccttttgcttcaatcaggtttggtggaggcaacctgaccagaaacacttcgctgctgcaagccagacaggaaaaagattccatgtcagataaggcaactgggctggtcttactttgcatcacctctgctttcctccactgccatcattaaacctcagctgtgacatgaaagacttaccggaccactgaaggtcttctgtaaaatataatgaagctgaaacctttggcctaagaagaaaatggaagtatgtgccactcgatttgtatttctgattaacaaataaacaggggtatttcctaaggtgaccatggttgaactttagctcatgaaagtggaaacattggtttaattttcaagagaattaagaaagtaaaagagaaattctgttatcaataacttgcaagtaattttttgtaaaagattgaattacagtaaacccatctttccttaacgaaaatttcctatgtttacagtctgtctattggtatgcaatcttgtaactttgataatgaacagtgagagatttttaaataaagcctctaaatatgttttgtcatttaataacatacagttttgtcacttttcaagtactttctgactcacatacagtagatcactttttactctgtgttaccattttgactggtcgtcattggcatggggtggatatagggcataggattacttgtctcagaagctgtcatagaatttcttgctgccaattaaaaaacctgtgttctttacacactacacgtataaatattgtaactgttcatctttgttgttttatcactgtaagcctgtcaaatcatagtatcctaagcatctgtaaatgctaattttgcatttttggaaaaacccattccttccaagctagtgtttttcattggctccaggtctaatttttcactgtggtccctggcagccagtcttttgaagtttaaagattacctgtctcttgactgcagtaccttttctttaatttttaccaaaaatatccagaggttactggagttcttattcaatataaggaaagtttgctgcactttattaccaagcctctgggattttaccagtcaaacatatttgtgcattacatttcatttcttgtgagctagctggctgtccatattgaatgttgacccatttgagtacgctaaaaggcttacagtatcagacacgatcatggttttagatcccataataaaaatgaatgtttttcttataaaaaattatacaaatgctgaagtgagattctactattgttcattgcttccttttctttttccttttgcgattttcactgattaatagcacatttcttcacaaaattagataaagttggtcaaagaccagatattctggaatggaaattgtaaagcttaatcaaaaagaatagccagtacagcatacaatctcagaaacttagaagcaagtagaaaataattggttgatgtaaacgaaagtgccattttagtaaaggcaggaaaaaaatagcaatatttgagttatgtaaggataaaaaatccactgacttgtatttttgcacaagaggctggtctgaatatgattgttcacattaagagtgtttattcgtcggttcattttggggattttcccccttgatgttttgacagattgaagtgagctttagtgagcaaaaggatcagaatgcagggaacactaagctgtgatgaagaaagtgtggtaaaaagccagagtagttttatacagacaaaaccagtgtcaggcctttgcagtaggcttgagtgaacttctgatctagatttgaaagtaaattttatgaagacattgcccatttttacttcctcattcattattgtaccagcatcatagctttattactctaatcccaggtaagtcaagcctacaatgccctagaggaagagtaaaaccagaaattcatgctggcttaaataatctatttttgtttcttttcatttgaatatttaaattttatggtttattaaaaaattaaataa

SEQ ID NO: 7 - protein, 197 aa, Homo sapiens
MHYPIGLLFDLLASSSALPWNITVHFKSFPEKDLLHCPSKDAIEAHFMSCMKEADALKHKSQVINEMQKKDHKQLWMGLQNDRFDQFWAINRKLMEYPAEENGFRYIPFRIYQTTTERPFIQKLFRPVAADGQLHTLGDLLKEVCPSAIDPEDGEKKNQVMIHGIEPMLETPLQWLSEHLSYPDNFLHISIIPQPTD